(19) United States
(12) Patent Application Publication
(10) Pub. No.: US 2025/0046419 A1
(43) Pub. Date: Feb. 6, 2025

(54) PATIENT ENGAGEMENT FOR WEARABLE MEDICAL DEVICES

(71) Applicant: ZOLL Medical Corporation, Chelmsford, MA (US)

(72) Inventors: Gregory M. Seese, Murrysville, PA (US); Erin L. McPeak, Pittsburgh, PA (US)

(21) Appl. No.: 18/836,130

(22) PCT Filed: Feb. 6, 2023
(86) PCT No.: PCT/US2023/062042

§ 371 Date: Aug. 6, 2024

Related U.S. Application Data
(60) Provisional application No. 63/267,638, filed on Feb. 7, 2022.

(51) Int. Cl. G16H 20/30; A61B 5/00; A61B 5/11; A61B 5/33; G16H 40/67

(52) U.S. Cl. CPC G16H 20/30; A61B 5/0006; A61B 5/1118; A61B 5/33; G16H 40/67

(57) ABSTRACT

A wearable cardiac monitoring/rehabilitative system configured to manage patient engagement incentives is provided. The system includes a wearable cardiac monitoring device, configured to continuously monitor a patient for arrhythmias, including one or more externally worn sensors and motion detectors. The system also includes a non-transitory server database and one or more server processors. The server processor(s) are configured to receive incentive criteria for a predetermined patient engagement incentive program for the patient, store a patient incentive data structure based on the incentive criteria, and receive from the device at least one ECG signal, motion data, and wear state data. The server processor(s) are also configured to determine whether the patient has satisfied or partially satisfied the incentive criteria based on the motion data and/or wear state data and if so, update the patient incentive data structure to indicate that the patient has earned a complete or partial incentive reward.

### CROSS-REFERENCE TO RELATED APPLICATIONS

This nonprovisional application claims priority to PCT Patent Application Serial No. PCT/US2023/062042, filed on Feb. 6, 2023, titled “PATIENT ENGAGEMENT FOR WEARABLE MEDICAL DEVICES,” which claims priority to U.S. Provisional Patent Application Ser. No. 63/267,638, filed on Feb. 7, 2022, titled “PATIENT ENGAGEMENT FOR WEARABLE MEDICAL DEVICES,” the entirety of each of which is hereby incorporated by reference.

### BACKGROUND

The present disclosure relates to a wearable cardiac monitoring and rehabilitative system configured to manage patient engagement.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients may be prescribed to wear cardiac monitoring and/or cardiac treatment devices for extended periods of time. Monitoring devices may monitor the patient's heart activity, and cardiac treatment devices may provide defibrillation shocks to the patient if an abnormal cardiac rhythm is detected. As such, patients are encouraged to comply with the prescription such that cardiac monitoring data can be gathered from the patient and/or the patient may be successfully treated if the patient suffers a treatable abnormal cardiac rhythm.

### SUMMARY

In one or more examples, a wearable cardiac monitoring and rehabilitative system configured to manage patient engagement incentives for ambulatory cardiac patients being monitored for cardiac arrhythmias is provided. The system includes a wearable cardiac monitoring device configured to continuously monitor and ambulatory cardiac patient for one or more arrhythmias. The device includes one or more externally worn sensors configured to output one or more physiological signals including at least one electrocardiogram (ECG) signal, one or more motion detectors configured to generate motion data indicative of physical activity performed by the ambulatory cardiac patient, and wear state detection circuitry configured to detect wear state data of the wearable cardiac monitoring device. The system also includes a non-transitory server database and one or more server processors in communication with the wearable cardiac monitoring device. The one or more server processors are configured to receive, from a clinician, incentive criteria for a predetermined patient engagement incentive program for the ambulatory cardiac patient; store, in the server database, a patient incentive data structure for the ambulatory cardiac patient based on the received incentive criteria; and receive from the wearable cardiac monitoring device over a period of time the at least one ECG signal, the motion data indicative of physical activity performed by the ambulatory cardiac patient, and the wear state data. The one or more server processors are further configured to determine whether the ambulatory cardiac patient has satisfied or partially satisfied the incentive criteria based on at least one of the received motion data or wear state data and, if the ambulatory cardiac patient has satisfied or partially satisfied the incentive criteria, update the patient incentive data structure to indicate that the ambulatory cardiac patient has earned a complete or partial incentive reward.

Implementations of the wearable cardiac monitoring and rehabilitative system configured to manage patient engagement incentives for ambulatory cardiac patients being monitored for cardiac arrhythmias can include one or more of the following features. The one or more server processors are further configured to determine a wear state for the wearable cardiac monitoring device, and the wear state includes an indication of whether the patient wore the wearable cardiac monitoring device. Determining whether the ambulatory cardiac patient has satisfied or partially satisfied the incentive criteria is further based on the received ECG signal. The system further includes a clinician-authorized user terminal. The one or more server processors are configured to receive the incentive criteria for the predetermined patient engagement incentive program via the clinician-authorized user terminal.

The incentive criteria for the predetermined patient engagement incentive program includes one or more physical activity goals for the ambulatory cardiac patient. The one or more physical activity goals include at least one of a number of steps for the ambulatory cardiac patient to perform, a number of minutes of physical activity for the ambulatory cardiac patient to perform, or a predetermined combination thereof. The incentive criteria for the predetermined patient engagement incentive program include an amount of time for the ambulatory cardiac patient to wear the wearable cardiac monitoring device. The incentive criteria for the for the predetermined patient engagement incentive program include a health goal for the ambulatory cardiac patient. The health goal includes at least one of a resting heart rate goal, an R-R interval goal, or a heart rate variability goal. The health goal includes a health improvement goal. The health improvement goal includes a number or percentage improvement over a starting health value.

The incentive criteria include a first set of incentive criteria, and the one or more server processors are further configured to receive, from the clinician, a second set of incentive criteria for the predetermined patient engagement incentive program for the ambulatory cardiac patient and update, in the server database, the patient incentive data structure for the ambulatory cardiac patient based on the received second set of incentive criteria. The one or more server processors are further configured to determine whether the ambulatory cardiac patient has satisfied or partially satisfied the second set of incentive criteria and, if the ambulatory cardiac patient has satisfied or partially satisfied the second set of incentive criteria, update the patient incentive data structure to indicate that the ambulatory cardiac patient has earned a complete or partial incentive reward. The second set of incentive criteria for the predetermined patient engagement incentive program include the ambulatory cardiac patient acknowledging a message sent to the ambulatory cardiac patient. The one or more server processors are further configured to receive message acknowledgement data for the ambulatory cardiac patient and determine whether the ambulatory cardiac patient has satisfied or partially satisfied the second set of incentive criteria based on the message acknowledgement data. Acknowledging the message sent to the ambulatory cardiac patient includes at least one of opening the message sent to the ambulatory cardiac patient, watching a video sent to the ambulatory cardiac patient, or completing a questionnaire sent to the ambulatory cardiac patient. The second set of incentive criteria for the predetermined patient incentive program include the ambulatory cardiac patient attending an appointment with a caregiver. The one or more server processors are further configured to receive appointment data for the ambulatory cardiac patient and determine whether the ambulatory cardiac patient has satisfied or partially satisfied the second set of incentive criteria based on the appointment data. The second set of incentive criteria for the predetermined patient engagement incentive program include the ambulatory cardiac patient sharing information related to at least one of a health status of the ambulatory cardiac patient or a status of the wearable cardiac monitoring device with a caregiver. The one or more server processor are further configured to receive information sharing data for the ambulatory cardiac patient and determine whether the ambulatory cardiac patient has satisfied or partially satisfied the second set of incentive criteria based on the information sharing data.

The one or more server processors are further configured to assign at least one predetermined weight to the incentive criteria. The at least one of a size or a type of the complete or partial incentive reward depends on the at least one weight of the incentive criteria. The weight assigned to each incentive criterion is associated with a risk score for the ambulatory cardiac patient's health if the ambulatory cardiac patient does not satisfy the incentive criterion. The incentive criteria are individualized to the ambulatory cardiac patient. The incentive criteria are based on demographics of the ambulatory cardiac patient. The incentive criteria are based on a predetermined classification of the ambulatory cardiac patient's health. The incentive criteria include a predetermined time period during which the ambulatory cardiac patient must complete one or more actions. The predetermined time period includes a user-configurable time period. The predetermined time period includes at least one of a day, 2 days, 3 days, 5 days, a week, 2 weeks, a month, 3 months, or 6 months. The one or more server processors are further configured to determine whether the ambulatory cardiac patient has satisfied or partially satisfied the incentive criteria by determining whether the ambulatory cardiac patient has satisfied a predetermined portion of the incentive criteria that is less than a total of the incentive criteria.

The wearable cardiac monitoring device further includes a garment configured to be worn about a torso of the ambulatory cardiac patient. The one or more externally worn sensors and the one or more motion detectors are configured to be mounted on the garment. The wearable cardiac monitoring device further includes at least one treatment electrode configured to deliver a cardioversion/defibrillation shock to the ambulatory cardiac patient. The wearable cardiac monitoring device further includes a controller configured to determine, using the at least one ECG signal, whether the ambulatory cardiac patient is experiencing a treatable arrhythmia and, in response to determining that the ambulatory cardiac patient is experiencing a treatable arrhythmia, deliver a cardioversion/defibrillation shock to the ambulatory cardiac patient via the at least one treatment electrode.

The wearable cardiac monitoring device includes an adhesive patch configured to be worn on a skin of the ambulatory cardiac patient for an extended period of time and a cardiac monitor configured to be mounted on the adhesive patch. The one or more externally worn sensors include at least one ECG electrode embedded in the adhesive patch and configured to output the at least one ECG signal. The cardiac monitor includes the one or more motion detectors.

In one or more examples, a wearable cardiac monitoring and rehabilitative system configured to manage patient engagement incentives for ambulatory cardiac patients being monitored for cardiac arrhythmias is provided. The system includes a non-transitory server database and one or more server processors in communication with a wearable cardiac monitoring device prescribed for an ambulatory cardiac patient. The wearable cardiac monitoring device is configured to continuously monitor the ambulatory cardiac patient for one or more arrhythmias. The one or more server processors are configured to receive, from a clinician, incentive criteria for a predetermined patient engagement incentive program for the ambulatory cardiac patient; store, in the server database, a patient incentive data structure for the ambulatory cardiac patient based on the received incentive criteria; and receive from the wearable cardiac monitoring device over a period of time at least one ECG signal, motion data indicative of physical activity performed by the ambulatory cardiac patient, and wear state data of the wearable cardiac monitoring device. The one or more server processors are further configured to determine whether the ambulatory cardiac patient has satisfied or partially satisfied the incentive criteria based on at least one of the received motion data or wear state data and, if the ambulatory cardiac patient has satisfied or partially satisfied the incentive criteria, update the patient incentive data structure to indicate that the ambulatory cardiac patient has earned a complete or partial incentive reward.

Implementations of this wearable cardiac monitoring and rehabilitative system configured to manage patient engagement incentives for ambulatory cardiac patients being monitored for cardiac arrhythmias can include one or more of the features discussed with respect to the wearable cardiac monitoring and rehabilitative system above.

In one or more examples, a non-transitory computer-readable medium storing sequences of instructions executable by at least one processor is provided. The sequences of instructions instruct the at least one processor to manage patient engagement incentives for ambulatory cardiac patients being monitored for cardiac arrhythmias. The sequences of instructions include instructions to receive, from a clinician, incentive criteria for a predetermined patient engagement incentive program for an ambulatory cardiac patient prescribed to wear a wearable cardiac monitoring device configured to continuously monitor the ambulatory cardiac patient for one or more arrhythmias; store, in a server database, a patient incentive data structure for the ambulatory cardiac patient based on the received incentive criteria; receive from the wearable cardiac monitoring device over a period of time at least one ECG signal, motion data indicative of physical activity performed by the ambulatory cardiac patient, and wear state data of the wearable cardiac monitoring device. The sequences of instructions also include instructions to determine whether the ambulatory cardiac patient has satisfied or partially satisfied the incentive criteria based on at least one of the received motion data or wear state data and, if the ambulatory cardiac patient has satisfied or partially satisfied the incentive criteria, update the patient incentive data structure to indicate that the ambulatory cardiac patient has earned a complete or partial incentive reward.

Implementations of this non-transitory computer-readable medium storing sequences of instructions executable by at least one processor can include one or more of the features discussed with respect to the wearable cardiac monitoring and rehabilitative system above.

In one or more examples, a method for managing patient engagement incentives for ambulatory cardiac patients being monitored for cardiac arrhythmias is performed. The method includes receiving, from a clinician, incentive criteria for a predetermined patient engagement incentive program for an ambulatory cardiac patient prescribed to wear a wearable cardiac monitoring device configured to continuously monitor the ambulatory cardiac patient for one or more arrhythmias. The method also includes storing, in a server database, a patient incentive data structure for the ambulatory cardiac patient based on the received incentive criteria and receiving from the wearable cardiac monitoring device over a period of time at least one ECG signal, motion data indicative of physical activity performed by the ambulatory cardiac patient, and a wear state data of the wearable cardiac monitoring device. The method further includes determining whether the ambulatory cardiac patient has satisfied or partially satisfied the incentive criteria based on at least one of the received motion data or wear state data and, if the ambulatory cardiac patient has satisfied or partially satisfied the incentive criteria, updating the patient incentive data structure to indicate that the ambulatory cardiac patient has earned a complete or partial incentive reward.

Implementations of the method for managing patient engagement incentives for ambulatory cardiac patients being monitored for cardiac arrhythmias can include one or more of the following features. The method includes determining a wear state for the wearable cardiac monitoring device. The wear state includes an indication of whether the patient wore the wearable cardiac monitoring device. Determining whether the ambulatory cardiac patient has satisfied or partially satisfied the incentive criteria is further based on the received ECG signal. Receiving the incentive criteria for the predetermined patient engagement incentive program includes receiving, from the clinician via a clinician-authorized user terminal, the incentive criteria for the predetermined patient engagement incentive program for the ambulatory cardiac patient prescribed to wear a wearable cardiac monitoring device configured to continuously monitor the ambulatory cardiac patient for one or more arrhythmias.

The incentive criteria for the predetermined engagement incentive program include one or more physical activity goals for the ambulatory cardiac patient. The one or more physical activity goals include at least one of a number of steps for the ambulatory cardiac patient to perform, a number of minutes of physical activity for the ambulatory cardiac patient to perform, or a predetermined combination thereof. The incentive criteria for the predetermined patient engagement incentive program include an amount of time for the ambulatory cardiac patient to wear the wearable cardiac monitoring device. The incentive criteria for the predetermined patient engagement incentive program include a health goal for the ambulatory cardiac patient. The health goal includes at least one of a resting heart rate goal, an R-R interval goal, or a heart rate variability goal. The health goal includes a health improvement goal. The health improvement goal includes a number or percentage improvement over a starting health value.

The incentive criteria include a first set of incentive criteria. The method further includes receiving, from the clinician, a second set of incentive criteria for the predetermined patient engagement incentive program for the ambulatory cardiac patient and updating, in the server database, the patient incentive data structure for the ambulatory cardiac patient based on the received second set of incentive criteria. The method further includes determining whether the ambulatory cardiac patient has satisfied or partially satisfied the second set of incentive criteria and, if the ambulatory cardiac patient has satisfied or partially satisfied the second set of incentive criteria, updating the patient incentive data structure to indicate that the ambulatory cardiac patient has earned a complete or partial incentive reward. The second set of incentive criteria for the predetermined patient engagement incentive program include the ambulatory cardiac patient acknowledging a message sent to the ambulatory cardiac patient. The method further includes receiving message acknowledgement data for the ambulatory cardiac patient and determining whether the ambulatory cardiac patient has satisfied or partially satisfied the second set of incentive criteria based on the message acknowledgement data. Acknowledging the message sent to the ambulatory cardiac patient includes at least one of opening the message sent to the ambulatory cardiac patient, watching a video sent to the ambulatory cardiac patient, or completing a questionnaire sent to the ambulatory cardiac patient. The second set of incentive criteria for the predetermined patient engagement incentive program include the ambulatory cardiac patient attending an appointment with a caregiver. The method further includes receiving appointment data for the ambulatory cardiac patient and determining whether the ambulatory cardiac patient has satisfied or partially satisfied the second set of incentive criteria based on the appointment data. The second set of incentive criteria for the predetermined patient engagement incentive program include the ambulatory cardiac patient sharing information related to at least one of a health status of the ambulatory cardiac patient or a status of the wearable cardiac monitoring device with a caregiver. The method further includes receiving information sharing data for the ambulatory cardiac patient and determining whether the ambulatory cardiac patient has satisfied or partially satisfied the second set of incentive criteria based on the information sharing data.

The method further includes assigning at least one predetermined weight to the incentive criteria. At least one of a size or a type of the complete or partial incentive reward depends on the at least one weight of the incentive criteria. The weight assigned to each incentive criterion is associated with a risk score for the ambulatory cardiac patient's health if the ambulatory cardiac patient does not satisfy the incentive criterion. The incentive criteria are individualized to the ambulatory cardiac patient. The incentive criteria are based on demographics of the ambulatory cardiac patient. The incentive criteria are based on a predetermined classification of the ambulatory cardiac patient's health.

The incentive criteria include a predetermined time period during which the ambulatory cardiac patient must complete one or more actions. The predetermined time period includes a user-configurable time period. The predetermined time period includes at least one of a day, 2 days, 3 days, 5 days, a week, 2 weeks, a month, 3 months, or 6 months. Determining whether the ambulatory cardiac patient has satisfied or partially satisfied the incentive criteria includes determining whether the ambulatory cardiac patient has satisfied a predetermined proportion of the incentive criteria that is less than a total of the incentive criteria based on at least one of the received motion data or wear state data.

The wearable cardiac monitoring device further includes one or more externally worn sensors configured to output one or more physiological signals including the at least one ECG signal. The wearable cardiac monitoring device further includes one or more motion detectors configured to generate motion data indicative of physical activity performed by the ambulatory cardiac patient. The wearable cardiac monitoring device further includes a garment configured to be worn about a torso of the ambulatory cardiac patient. The one or more externally worn sensors and the one or more motion detectors configured to be mounted on the garment. The wearable cardiac monitoring device further includes at least one treatment electrode configured to deliver a cardioversion/defibrillation shock to the ambulatory cardiac patient. The wearable cardiac monitoring device further includes a controller. The method further includes determining, using the at least one ECG signal, whether the ambulatory cardiac patient is experiencing a treatable arrhythmia and, in response to determining that the ambulatory cardiac patient is experiencing a treatable arrhythmia, delivering a cardioversion/defibrillation shock to the ambulatory cardiac patient via the at least one treatment electrode.

The wearable cardiac monitoring device further includes an adhesive patch configured to be worn on skin of the ambulatory cardiac patient for an extended period of time and a cardiac monitor configured to be mounted on the adhesive patch. The one or more externally worn sensors include at least one ECG electrode embedded in the adhesive patch and configured to output the at least one ECG signal. The cardiac monitor includes the one or more motion detectors.

In one or more examples, a wearable cardiac monitoring and rehabilitative system configured to monitor patient engagement among ambulatory cardiac patients being monitored for cardiac arrhythmias is provided. The system includes a wearable cardiac monitoring device configured to continuously monitor an ambulatory cardiac patient for one or more arrhythmias. The device is configured to generate one or more of an electrocardiogram (ECG) signal, motion data indicative of physical activity performed by the ambulatory cardiac patient, or wear state data of the wearable cardiac monitoring device. The system also includes a non-transitory server database and one or more server processors in communication with the wearable cardiac monitoring device. The one or more server processors are configured to receive, from a clinician, incentive criteria for a predetermined patient engagement incentive program for the ambulatory cardiac patient; store, in the server database, a patient incentive data structure for the ambulatory cardiac patient based on the received incentive criteria; and receive from the wearable cardiac monitoring device over a period of time the one or more of the ECG signal, the motion data indicative of physical activity performed by the ambulatory cardiac patient, or the wear state data of the wearable cardiac monitoring device. The one or more server processors are further configured to update, based on the received one or more of the ECG signal, motion data, or wear state data, the patient incentive data structure to indicate the ambulatory cardiac patient's progress towards earning incentive rewards; retrieve, from the server database, incentive rewards progress information of a predetermined cohort of other ambulatory cardiac patients; and provide an output indicating the ambulatory cardiac patient's progress towards earning incentive rewards relative to the incentive rewards progress information of the predetermined cohort of other ambulatory cardiac patients.

Implementations of the wearable cardiac monitoring and rehabilitative system configured to monitor patient engagement among ambulatory cardiac patients being monitored for cardiac arrhythmias can include one or more of the following features. The one or more server processors are further configured to determine a wear state for the wearable cardiac monitoring device. The wear state data includes an indication of whether the patient wore the wearable cardiac monitoring device. The system includes a clinician-authorized user terminal. The one or more server processors are configured to receive the incentive criteria for the predetermined patient engagement incentive program via the clinician-authorized user terminal.

The incentive criteria for the predetermined engagement incentive program include one or more physical activity goals for the ambulatory cardiac patient. The one or more physical activity goals include at least one of a number of steps for the ambulatory cardiac patient to perform, a number of minutes of physical activity for the ambulatory cardiac patient to perform, or a predetermined combination thereof. The incentive criteria for the predetermined patient engagement incentive program include an amount of time for the ambulatory cardiac patient to wear the wearable cardiac monitoring device. The incentive criteria for the predetermined patient engagement incentive program include a health goal for the ambulatory cardiac patient. The health goal includes at least one of a resting heart rate goal, an R-R interval goal, or a heart rate variability goal. The health goal includes a health improvement goal. The health improvement goal includes a number or percentage improvement over a starting health value.

The one or more server processors are further configured to assign at least one predetermined weight to the incentive criteria. At least one of a size or type of the complete or partial incentive reward depends on the at least one weight of the incentive criteria. The weight assigned to each incentive criterion is associated with a risk score for the ambulatory cardiac patient's health if the ambulatory cardiac patient does not satisfy the incentive criteria. The incentive criteria are individualized to the ambulatory cardiac patient. The incentive criteria are based on demographics of the ambulatory cardiac patient. The incentive criteria are based on a predetermined classification of the ambulatory cardiac patient's health.

The one or more server processors are further configured to adjust the output indicating the ambulatory cardiac patient's progress towards earning incentive rewards relative to the incentive rewards progress information of the predetermined cohort of other ambulatory cardiac patients to account for a health status of the ambulatory cardiac patient relative to health statuses of the predetermined cohort of other ambulatory cardiac patients. The health status of the ambulatory cardiac patient includes at least one of a heart failure classification of the ambulatory cardiac patient or a time elapsed from a prior myocardial infarction event experienced by the ambulatory cardiac patient. The health statuses of the predetermined cohort of other ambulatory cardiac patients includes at least one of heart failure classifications for the predetermined cohort of other ambulatory cardiac patients or prior myocardial infarction events experienced by the predetermined cohort of other ambulatory cardiac patients.

The one or more server processors are further configured to adjust the output indicating the ambulatory cardiac patient's progress towards earning incentive rewards relative to the incentive rewards progress information of the predetermined cohort of other ambulatory cardiac patients to account for at least one demographic of the ambulatory cardiac patient relative to demographics of the predetermined cohort of other ambulatory cardiac patients. The at least one demographic of the ambulatory cardiac patient includes at least one of an age or a gender of the ambulatory cardiac patient. The demographics of the predetermined cohort of other ambulatory cardiac patients include at least one of ages or genders of the predetermined cohort of other ambulatory cardiac patients. The output includes a visual representation indicating the ambulatory cardiac patient's progress towards earning incentive rewards relative to the incentive rewards progress information of the predetermined cohort of other ambulatory cardiac patients. The one or more server processors are further configured to transmit the visual representation to a patient user device. The one or more server processors are further configured to transmit the visual representation to a clinician-authorized user terminal. The output indicating the ambulatory cardiac patient's progress towards earning incentive rewards relative to the incentive rewards progress information of the predetermined cohort of other ambulatory cardiac patients includes a trend of incentive rewards earned by the ambulatory cardiac patient over a time period relative to a trend of incentive rewards earned by the predetermined cohort of other ambulatory cardiac patients over the time period.

The one or more server processors are further configured to receive, from the clinician, aggregate incentive criteria for a patient population comprising the ambulatory cardiac patient. The aggregate incentive criteria define an aggregate goal for the patient population. The one or more server processors are further configured to store, in the server database, an aggregate incentive data structure for the patient population based on the aggregate incentive criteria. The one or more server processors are further configured to update, based on one or more of motion data of the patient population or wear state data of the patient population, the aggregate incentive data structure to indicate the patient population's progress towards earning incentive rewards and provide an aggregate output indicating the patient population's progress towards completing the aggregate goal. The patient population further includes the predetermined cohort of other ambulatory cardiac patients. The one or more server processors are further configured to receive an input from the ambulatory cardiac patient opting in to being compared relative to the predetermined cohort of other ambulatory cardiac patients and provide the output indicating the ambulatory cardiac patient's progress towards earning incentive rewards relative to the incentive rewards progress information of the predetermined cohort of other ambulatory cardiac patients after receiving the input from the ambulatory cardiac patient opting in to being compared relative to the predetermined cohort of other ambulatory cardiac patients.

The one or more server processors are further configured to identify the predetermined cohort of other ambulatory cardiac patients. The one or more server processors are further configured to identify the predetermined cohort of other ambulatory cardiac patients based on a demographic shared between the ambulatory cardiac patient and the cohort of other ambulatory cardiac patients. The one or more server processors are further configured to identify the predetermined cohort of other ambulatory cardiac patients based on a health status shared between the ambulatory cardiac patient and the cohort of other ambulatory cardiac patients. The one or more server processors are further configured to identify the predetermined cohort of other ambulatory cardiac patients based on a date range during which the ambulatory cardiac patient and the cohort of other ambulatory cardiac patients received their respective wearable cardiac monitoring devices. The one or more server processors are further configured to receive a request from the ambulatory cardiac patient to share the ambulatory cardiac patient's progress towards earning incentive rewards with a second ambulatory cardiac patient from the predetermined cohort of other ambulatory cardiac patients and provide the ambulatory cardiac patient's progress towards earning incentive rewards to the second ambulatory cardiac patient from the predetermined cohort of other ambulatory cardiac patients.

The wearable cardiac monitoring device includes a garment configured to be worn about a torso of the ambulatory cardiac patient. The wearable cardiac monitoring device further includes one or more externally worn sensors configured to generate the ECG signal and one or more motion detectors configured to generate the motion data indicating of physical activity performed by the ambulatory cardiac patient. The one or more externally worn sensors and the one or more motion detectors are configured to be mounted on the garment. The wearable cardiac monitoring device further includes at least one treatment electrode configured to deliver a cardioversion/defibrillation shock to the ambulatory cardiac patient. The wearable cardiac monitoring device is configured to generate at least the ECG signals. The wearable cardiac monitoring device further includes a controller configured to determine, using the ECG signal, whether the ambulatory cardiac patient is experiencing a treatable arrhythmia and, in response to determining that the ambulatory cardiac patient is experiencing the treatable arrhythmia, deliver a cardioversion/defibrillation shock to the ambulatory cardiac patient via the at least one treatment electrode.

The wearable cardiac monitoring device includes an adhesive patch configured to be worn on skin of the ambulatory cardiac patient for an extended period of time and a cardiac monitor configured to be mounted on the adhesive patch. The wearable cardiac monitoring device further includes at least one ECG electrode embedded in the adhesive patch and configured to generate the ECG signal. The cardiac monitor includes at least one motion detector configured to generate the motion data indicative of physical activity performed by the ambulatory cardiac patient.

In one or more examples, a wearable cardiac monitoring and rehabilitative system configured to monitor patient engagement among ambulatory cardiac patients being monitored for cardiac arrhythmias is provided. The system includes a non-transitory server database and one or more server processors in communication with a wearable cardiac monitoring device prescribed for an ambulatory cardiac patient. The wearable cardiac monitoring device is configured to continuously monitor the ambulatory cardiac patient for one or more arrhythmias. The one or more server processors are configured to receive, from a clinician, incentive criteria for a predetermined patient engagement incentive program for the ambulatory cardiac patient; store, in the server database, a patient incentive data structure for the ambulatory cardiac patient based on the received incentive criteria; and receive from the wearable cardiac monitoring device over a period of time one or more of an ECG signal, motion data indicative of physical activity performed by the ambulatory cardiac patient, or wear state data of the wearable cardiac monitoring device. The one or more server processors are further configured to update, based on the received one or more of the ECG signal, motion data, or wear state data, the patient incentive data structure to indicate the ambulatory cardiac patient's progress towards earning incentive rewards; retrieve, from the server database, incentive rewards progress information of a predetermined cohort of other ambulatory cardiac patients; and provide an output indicating the ambulatory cardiac patient's progress towards earning incentive rewards relative to the incentive rewards progress information of the predetermined cohort of other ambulatory cardiac patients.

Implementations of this wearable cardiac monitoring and rehabilitative system configured to monitor patient engagement among ambulatory cardiac patients being monitored for cardiac arrhythmias can include one or more of the features discussed with respect to the wearable cardiac monitoring and rehabilitative system above.

In one or more examples, a non-transitory computer-readable medium storing sequences of instructions executable by at least one processor. The sequences of instructions instruct the at least one processor to monitor patient engagement among ambulatory cardiac patients being monitored for cardiac arrhythmias. The sequences of instructions include instructions to receive, from a clinician, incentive criteria for a predetermined patient engagement incentive program for an ambulatory cardiac patient prescribed to wear a wearable cardiac monitoring device configured to continuously monitor the ambulatory cardiac patient for one or more arrhythmias; store, in a server database, a patient incentive data structure for the ambulatory cardiac patient based on the received incentive criteria; and receive from the wearable cardiac monitoring device over a period of time one or more of an ECG signal, motion data indicative of physical activity performed by the ambulatory cardiac patient, or wear state data of the wearable cardiac monitoring device. The sequences of instructions further include instructions to update, based on the received one or more of the ECG signal, motion data, or wear state data, the patient incentive data structure to indicate the ambulatory cardiac patient's progress towards earning incentive rewards; retrieve, from the server database, incentive rewards progress information of a predetermined cohort of other ambulatory cardiac patients; and provide an output indicating the ambulatory cardiac patient's progress towards earning incentive rewards relative to the incentive rewards progress information of the predetermined cohort of other ambulatory cardiac patients.

Implementations of this non-transitory computer-readable medium storing sequences of instructions executable by at least one processor can include one or more of the features discussed with respect to the wearable cardiac monitoring and rehabilitative system above.

In one or more examples, a method for monitoring patient engagement among ambulatory cardiac patients being monitored for cardiac arrhythmias is provided. The method includes receiving, from a clinician, incentive criteria for a predetermined patient engagement incentive program for an ambulatory cardiac patient prescribed to wear a wearable cardiac monitoring device configured to continuously monitor the ambulatory cardiac patient for one or more arrhythmias; storing, in a server database, a patient incentive data structure for the ambulatory cardiac patient based on the received incentive criteria; and receiving from the wearable cardiac monitoring device over a period of time one or more of an ECG signal, motion data indicative of physical activity performed by the ambulatory cardiac patient, or wear state data of the wearable cardiac monitoring device. The method further includes updating, based on the received one or more of the ECG signal, motion data, or wear state data, the patient incentive data structure to indicate the ambulatory cardiac patient's progress towards earning incentive rewards; retrieving, from the server database, incentive rewards progress information of a predetermined cohort of other ambulatory cardiac patients; and providing an output indicating the ambulatory cardiac patient's progress towards earning incentive rewards relative to the incentive rewards progress information of the predetermined cohort of other ambulatory cardiac patient.

Implementations of the method for monitoring patient engagement among ambulatory cardiac patients being monitored for cardiac arrhythmias can include one or more of the following features. The method further includes determining a wear state for the wearable cardiac monitoring device. The wear state data includes an indication of whether the patient wore the wearable cardiac monitoring device. Receiving the incentive criteria for the predetermined patient engagement incentive program includes receiving, from the clinician via a clinician-authorized user terminal, the incentive criteria for the predetermined patient engagement incentive program for the ambulatory cardiac patient prescribed to wear the wearable cardiac monitoring device configured to continuously monitor the ambulatory cardiac patient for one or more arrhythmias.

The incentive criteria for the predetermined engagement incentive program include one or more physical activity goals for the ambulatory cardiac patient. The one or more physical activity goals include at least one of a number of steps for the ambulatory cardiac patient to perform, a number of minutes of physical activity for the ambulatory cardiac patient to perform, or a predetermined combination thereof. The incentive criteria for the predetermined patient engagement incentive program include an amount of time for the ambulatory cardiac patient to wear the wearable cardiac monitoring device. The incentive criteria for the predetermined patient engagement incentive program include a health goal for the ambulatory cardiac patient. The health goal includes at least one of a resting heart rate goal, an R-R interval goal, or a heart rate variability goal. The health goal includes a health improvement goal. The health improvement goal includes a number or percentage improvement over a starting health value.

The method further includes assigning at least one predetermined weight to the incentive criteria. At least one of a size or type of the complete or partial incentive reward depends on the at least one weight of the incentive criteria. The weight assigned to each incentive criterion is associated with a risk score for the ambulatory cardiac patient's health if the ambulatory cardiac patient does not satisfy the incentive criteria. The incentive criteria are individualized to the ambulatory cardiac patient. The incentive criteria are based on demographics of the ambulatory cardiac patient. The incentive criteria are based on a predetermined classification of the ambulatory cardiac patient's health.

The method further includes adjusting the output indicating the ambulatory cardiac patient's progress towards earning incentive rewards relative to the incentive rewards progress information of the predetermined cohort of other ambulatory cardiac patients to account for a health status of the ambulatory cardiac patient relative to health statuses of the predetermined cohort of other ambulatory cardiac patients. The health status of the ambulatory cardiac patient includes at least one of a heart failure classification of the ambulatory cardiac patient or a time elapsed from a prior myocardial infarction event experienced by the ambulatory cardiac patient. The health status of the predetermined cohort of other ambulatory cardiac patients includes at least one of heart failure classifications for the predetermined cohort of other ambulatory cardiac patients or prior myocardial infarction events experienced by the predetermined cohort of other ambulatory cardiac patients.

The method further includes adjusting the output indicating the ambulatory cardiac patient's progress towards earning incentive rewards relative to the incentive rewards progress information of the predetermined cohort of other ambulatory cardiac patients to account for at least one demographic of the ambulatory cardiac patient relative to demographics of the predetermined cohort of other ambulatory cardiac patients. The at least one demographic of the ambulatory cardiac patient includes at least one of an age or a gender of the ambulatory cardiac patient. The demographics of the predetermined cohort of other ambulatory cardiac patients include at least one of ages or genders of the predetermined cohort of other ambulatory cardiac patients.

The output includes a visual representation indicating the ambulatory cardiac patient's progress towards earning incentive rewards relative to the incentive rewards progress information of the predetermined cohort of other ambulatory cardiac patients. The method further includes transmitting the visual representation to a patient user device. The method further includes transmitting the visual representation to a clinician-authorized user terminal. The output indicating the ambulatory cardiac patient's progress towards earning incentive rewards relative to the incentive rewards progress information of the predetermined cohort of other ambulatory cardiac patients includes a trend of incentive rewards earned by the ambulatory cardiac patients over a time period relative to a trend of incentive rewards earned by the predetermined cohort of other ambulatory cardiac patients over the time period.

The method further includes receiving, from the clinician, aggregate incentive criteria for a patient computing including the ambulatory cardiac patient. The aggregate incentive criteria define an aggregate goal for the patient population. The method further includes storing, in the server database, an aggregate data structure for the patient population based on the aggregate incentive criteria. The method further includes updating, based on one or more of motion data of the patient population or wear state data of the patient population, the aggregate incentive data structure to indicate the patient population's progress towards earning incentive rewards and providing an aggregate output indicating the patient population's progress towards completing the aggregate goal. The patient population further includes the predetermined cohort of other ambulatory cardiac patients. The method further includes receiving an input from the ambulatory cardiac patient opting in to being compared relative to the predetermined cohort of other ambulatory cardiac patients and providing the output indicating the ambulatory cardiac patient's progress towards earning incentive rewards relative to the incentive rewards progress information of the predetermined cohort of other ambulatory cardiac patients after receiving the input from the ambulatory cardiac patient opting in to being compared relative to the predetermined cohort of other ambulatory cardiac patients.

The method further includes identifying the predetermined cohort of other ambulatory cardiac patients. Identifying the predetermined cohort of other ambulatory cardiac patients includes identifying the predetermined cohort of other ambulatory cardiac patients based on a demographic shared between the ambulatory cardiac patient and the cohort of other ambulatory cardiac patients. Identifying the predetermined cohort of other ambulatory cardiac patients includes identifying the predetermined cohort of other ambulatory cardiac patients based on a health status shared between the ambulatory cardiac patient and the cohort of other ambulatory cardiac patients. Identifying the predetermined cohort of other ambulatory cardiac patients includes identifying the predetermined cohort of other ambulatory cardiac patients based on a date range during which the ambulatory cardiac patient and the cohort of other ambulatory cardiac patients received their respective wearable cardiac monitoring devices. The method further includes receiving a request from the ambulatory cardiac patient to share the ambulatory cardiac patient's progress towards earning incentive rewards with a second ambulatory cardiac patient from the predetermined cohort of other ambulatory cardiac patients and providing the ambulatory cardiac patient's progress towards earning incentive rewards to the second ambulatory cardiac patient from the predetermined cohort of other ambulatory cardiac patients.

The wearable cardiac monitoring device includes a garment configured to be worn about a torso of the ambulatory cardiac patient. The wearable cardiac monitoring device further includes one or more externally worn sensors configured to generate the ECG signal and one or more motion detectors configured to generate motion data indicative of physical activity performed by the ambulatory cardiac patient. The one or more externally worn sensors and the one or more motion detectors configured to be mounted on the garment. The wearable cardiac monitoring device further includes at least one treatment electrode configured to deliver a cardioversion/defibrillation shock to the ambulatory cardiac patient. The wearable cardiac monitoring device is configured to generate at least the ECG signal. The wearable cardiac monitoring device further includes a controller. The method further includes determining, using the at least one ECG signal, whether the ambulatory cardiac patient is experiencing a treatable arrhythmia and, in response to determining that the ambulatory cardiac patient is experiencing a treatable arrhythmia, delivering a cardioversion/defibrillation shock to the ambulatory cardiac patient via the at least one treatment electrode.

The wearable cardiac monitoring device further includes an adhesive patch configured to be worn on skin of the ambulatory cardiac patient for an extended period of time and a cardiac monitor configured to be mounted on the adhesive patch. The one or more externally worn sensors include at least one ECG electrode embedded in the adhesive patch and configured to generate the ECG signal. The cardiac monitor includes at least one motion detector configured to generate the motion data indicative of physical activity performed by the ambulatory cardiac patient.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example wearable cardiac monitoring and rehabilitative system.

FIG. 2A depicts an example sample process flow for managing patient engagement incentives for ambulatory cardiac patients being monitored for cardiac arrhythmias.

FIG. 2B depicts another example sample process flow for managing patient engagement incentives for ambulatory cardiac patients being monitored for cardiac arrhythmias.

FIG. 2C depicts another example sample process flow for managing patient engagement incentives for ambulatory cardiac patients being monitored for cardiac arrhythmias.

FIG. 3 depicts another example process flow for managing patient engagement incentives for ambulatory cardiac patients being monitored for cardiac arrhythmias.

FIG. 4 depicts an example wearable cardiac monitoring device.

FIG. 5A depicts an example electronic architecture for a wearable cardiac monitoring device.

FIG. 5B depicts another example electronic architecture for a wearable cardiac monitoring device.

FIG. 6 depicts another example wearable cardiac monitoring device.

FIG. 7 depicts an example of a cardiac monitor being attached to an adhesive patch.

FIG. 8 depicts an example of a cardiac monitor and adhesive patch placed on a patient.

FIG. 9 depicts another example of a cardiac monitor and an adhesive patch placed on a patient.

FIG. 10 depicts an example exploded view of a cardiac monitor.

FIG. 11A depicts an example electronic architecture for a cardiac monitor.

FIG. 11B depicts an example process flow for a patient receiving and using a wearable cardiac monitoring device.

FIG. 12 depicts an example user interface for entering patient incentive criteria.

FIG. 13 depicts another example user interface for entering patient incentive criteria.

FIG. 14 depicts an example user interface for displaying rewards to a patient.

FIG. 15 depicts another example user interface for displaying rewards to a patient.

FIG. 16 depicts another example user interface for displaying rewards to a patient.

FIG. 17A depicts an example electronic architecture for a wearable cardiac monitoring device.

FIG. 17B depicts an example electronic architecture for a remote server in communication with a wearable cardiac monitoring device.

FIG. 18A depicts an example user interface.

FIG. 18B depicts another example user interface.

FIG. 18C depicts another example user interface.

FIG. 18D depicts another example user interface.

FIG. 18E depicts another example user interface.

FIG. 18F depicts another example user interface.

FIG. 19 depicts another example wearable cardiac monitoring device.

FIG. 20 depicts another example wearable cardiac monitoring device.

### DETAILED DESCRIPTION

Wearable medical devices, such as cardiac event monitoring and/or treatment devices, are used in clinical or outpatient settings to monitor and/or record ECG and other physiological signals of a patient. These ECG and other physiological signals can be used to monitor for arrhythmias, and, in example devices described herein, provide treatment such as defibrillation, cardioversion, or pacing shocks in the event of life-threatening arrhythmias. As such, features, techniques, and systems are described herein that encourage and/or verify that the patient is complying with device use guidelines by continuously wearing a prescribed wearable medical device. Such features can be important to the ambulatory patient's health, as the monitoring and/or treatment functions can only be performed while the patient is wearing the device. Further, features, techniques, and systems are described herein that encourage and/or verify that the patient understands how to use the wearable medical device (e.g., how to assemble the device, disassemble the device, clean the device, charge the device, etc.). Such features can also be important to the device being functional while the patient is wearing it. Additionally, a clinician or other caregiver for the patient may prescribe that the patient maintain a certain health level and/or make improvements to their health, such as by taking a certain number of steps in a day, by exercising a certain amount of time in a day or a week, by getting a certain number of hours of sleep per night, and so on. Further, a patient may need to attend follow-up appointments and/or provide a clinician or other caregiver for the patient with information about the patient's health or the status of the wearable medical device. Features, techniques, and systems are described herein to encourage and/or verify patient compliance with some or all of the foregoing guidelines.

As such, this disclosure relates to a wearable cardiac monitoring and rehabilitative system configured to manage patient engagement incentives for ambulatory cardiac patients being monitored for cardiac arrhythmias. A patient is prescribed a wearable cardiac monitoring device configured to continuously monitor an ambulatory cardiac patient for arrhythmias. The wearable cardiac monitoring device includes one or more externally worn sensors configured to output physiological signals including an ECG signal, one or more motion detectors configured to generate motion data indicative of physical activity performed by the ambulatory cardiac patient, and/or wear state detection circuitry configured to detect wear state data of the wearable cardiac monitoring device. The wearable cardiac monitoring device may include monitoring-only functionalities, e.g., devices and systems such as arrhythmia monitoring system (AMS) or heart failure management system (HFMS) devices. In some examples, the wearable cardiac monitoring device may include treatment functionalities, e.g., devices and systems such as a wearable cardioverter defibrillator (WCD). For example, the wearable cardiac monitoring device may provide a therapeutic shock (e.g., defibrillation, cardioversion, and/or pacing shock) to the patient upon detecting a treatable arrhythmia.

The wearable cardiac monitoring device is configured to transmit the physiological signals (e.g., including the ECG signal), the motion data, and/or the wear state data to a remote server. Separately, the remote server is also configured to receive, from a clinician or other caregiver, incentive criteria for the patient. The incentive criteria define one or more goals for the patient to accomplish in connection with one or more patient engagement initiatives. Such patient engagement initiatives can include encouraging and/or verifying a certain level of device use period per day, per week, or other predetermined period. Such patient engagement initiatives can include encouraging and/or verifying a certain level of physical activity performed by the patient per day, per week, or other predetermined period. Such patient engagement initiatives can include encouraging and/or verifying that the patient learn certain information about best practices, uses, or guidelines relating to the wearable device. Examples of goals include acknowledging messages sent to the patient (e.g., text messages or emails reminding the patient of an appointment, a message with a questionnaire, a message with an educational video for the patient to watch), viewing an educational chapter or video, viewing a landing page associated with the wearable cardiac monitoring device (e.g., with information about the device and/or information about the patient engagement incentive program), completing questionnaires, meeting wear compliance requirements for the wearable cardiac monitoring device (e.g., wearing the wearable cardiac monitoring device for a certain amount of time each day), meeting a step count goal, meeting an exercise goal, meeting a physiological goal (e.g., maintaining heart rate, R-R interval, heart rate variability, etc. within a target range), meeting a sleep goal (e.g., sleeping a certain amount of hours per day), attending a follow-up appointment, and/or the like. The patient can then earn rewards in a patient engagement incentive program by meeting the goals set by the clinician or other caregiver through the incentive criteria.

Upon receiving the incentive criteria from the clinician or other caregiver, the remote server is configured to store, in a server database, a patient incentive data structure for the ambulatory cardiac patient based on the received incentive criteria. The patient incentive data structure may include, for instance, the goals set for the patient, rewards that the patient can earn by meeting these goals, a timeframe for meeting the goals, a maximum amount of rewards that the patient can earn by meeting each goal, and/or the like. The remote server is further configured to determine, using the received physiological signals, motion data, and/or wear state data, whether the ambulatory cardiac patient has satisfied or partially satisfied the incentive criteria. If the patient has satisfied and/or partially satisfied the incentive criteria, the remote server is configured to update the patient incentive data structure to indicate the rewards and/or partial rewards that the patient has earned.

Additionally, the remote server may group the patient with a predetermined cohort of other ambulatory cardiac patients, for example, based on the patients sharing a clinician, the patients being located in the same geographical area, the patients having similar demographics, the patients having similar health levels, and/or the like. The remote server may update the patient incentive data structure to indicate the patient's progress towards earning incentive rewards (e.g., including complete and partial rewards the patient has earned, including rewards milestones the patient has met, including the patient's progress towards rewards the patient has not yet earned, and/or the like) and retrieve similar incentive rewards progress information for the predetermined cohort of other patients. The remote server may then provide an output indicating the patient's progress towards incentive rewards relative to the incentive rewards progress information of the predetermined cohort of other ambulatory cardiac patients. For example, the remote server may provide a visual representation of the patient's progress towards earning various rewards and reward milestones versus the progress of the predetermined cohort of other patients.

In one example use case, a clinician or other caregiver prescribes that a patient at risk of heart failure wear a wearable cardiac monitoring device for a certain amount of time (e.g., until the patient is scheduled for a surgery to receive an implantable cardiac defibrillator). The wearable cardiac monitoring device may include monitoring functions and therapy functions such that the wearable cardiac monitoring device can monitor for cardiac arrhythmias and provide a therapeutic shock to the patient in response to detecting a treatable arrhythmia. The caregiver also sets a rehabilitative program for the patient that includes the patient getting 30 minutes of exercise and completing at least 5,000 steps per day. The caregiver sends these rehabilitative goals to the remote server (e.g., via an authorized user interface or terminal), and the remote server generates a patient incentive data structure that includes the goals of 30 minutes of exercise and at least 5,000 steps per day.

As the patient wears the wearable cardiac monitoring device, the wearable cardiac monitoring device transmits ECG signals and motion data to the remote server, where the motion data includes accelerometer counts and posture information for the patient. Using the ECG signals, accelerometer counts, and posture information, the remote server determines how much time per day the patient was active (e.g., using the patient's heart rate determined from the ECG signals and accelerometer counts) and how many steps per day the patient took (e.g., using the patient's accelerometer counts and posture information). The remote server then determines each day whether the patient met the 30 minutes of exercise and 5,000 steps per day goal. For each day each goal is met, the remote server updates the patient incentive data structure to indicate associated rewards earned for the met goals (e.g., 2 points per goal per day). The patient can view the rewards the patient has earned at any given time on a patient engagement application running on their smartphone. While the predetermined incentive rewards are described here as “points” it is understood that other types of rewards indicia such as “ribbons” or credits may be implemented.

In another example use case, a clinician or other caregiver prescribes that a patient who has complained of chest pain wear a wearable cardiac monitoring device for a certain amount of time (e.g., 15 days, 30 days, 60 days, 90 days). The wearable cardiac monitoring device is configured to monitor physiological attributes of the patient over the prescribed amount of time, such as the patient's ECG and motion, to determine if the patient has been experiencing cardiac arrhythmias. The remote server sets as, a default incentive criteria for a patient engagement incentive program, that the patient wear the wearable cardiac monitoring device for at least 22 hours each day. The remote server also sets as a default that the patient can earn 1 “ribbon” (or points, or credits, or other predetermined rewards indicia) if the patient wears the wearable cardiac monitoring device for 20 hours in a day and that the patient can earn 2 ribbons if the patient wears the wearable cardiac monitoring device for the full 22 hours in a day. The remote server also receives incentive criteria from the patient's clinician or other caregiver asking that the patient fill out a daily health questionnaire, which the remote server sets from a default that the patient can earn 1 ribbon per day from completing. The incentive criteria further includes a goal for the patient to get at least 7 hours of sleep per night, as well as a request from the physician to increase the amount of rewards ribbons per day from a default of 2 ribbons per day up to 3 ribbons per day for the goal of getting at least 7 hours of sleep per night. The remote server generates a patient incentive data structure including these goals and associated rewards.

The remote server receives data related to the monitored physiological attributes of the patient, including ECG signals and motion data. The remote server also receives wear state data from an impedance sensor incorporated in the wearable cardiac monitoring device, where the impedance sensor is configured to transmit a falloff signal into the patient and receive the signal back from the patient, provided that the patient is wearing the wearable cardiac monitoring device. From the ECG signals, motion data, and wear state data, the remote server determines how many hours per day the patient has been wearing the wearable cardiac monitoring device (e.g., from the wear state data) and how many hours per day the patient has been sleeping (e.g., from the patient's heart rate determined from the ECG signals and from the motion data). Additionally, the remote server receives daily data (e.g., from the clinician or other caregiver, from an application running on the patient's phone, from a server hosting a website associated with the patient engagement incentive program, etc.) indicating whether the patient sent in the questionnaire for that day. The remote server determines, each day, which of the patient's goals have been met (or partially met, in the case of wear compliance) and updates the patient incentive data structure to indicate the complete and/or partial rewards that the patient has earned. The remote server also generates a weekly output summarizing the patient's progress towards earning the rewards and transmits the weekly output to the clinician or other caregiver to review.

In another example use case, a caregiver prescribes for a group of patients at risk of heart failure that each patient wear a wearable cardiac monitoring device for a certain amount of time. The caregiver also sets health goals for each patient and transmits the incentive criteria defining the health goals to the remote server. The remote server generates a patient incentive data structure for each of the patients, storing in the patient incentive data structure the respective patient's health goals and associated rewards. The remote server receives physiological data from each of the wearable cardiac monitoring devices, such as ECG signals, motion data, bioacoustics data, etc., which the remote server uses to periodically determine (e.g., daily, weekly, monthly, etc.) if each of the patients has met their associated health goals. As the patients meet health goals and earn rewards, the remote server updates the patient incentive data structures to indicate the rewards earned. Additionally, for each patient, the remote server generates visual representations that the patient can view by accessing a patient engagement dashboard (e.g., via an application running on a smartphone or tablet computer, by logging into the dashboard via a browser, etc.) showing the patient's progress towards earning rewards, as well as how the patient's progress compares to the progress of the average patient of the group of patients and the progress of the top three earners of the group of patients.

The wearable cardiac monitoring and rehabilitative system described herein may provide several advantages over prior art systems. By providing a patient with a patient engagement incentive program that rewards the patient for understanding and complying with a prescription to wear a wearable cardiac monitoring device, the patient may be more likely to comply with the prescription, thereby ensuring better monitoring and/or treatment can be provided to the patient. Additionally, as described above, the patient engagement incentive program can reward patients for taking actions under a health improvement plan set by the patient's clinician or other caregiver, as well as performing other actions likely to improve the patient's health, such as attending follow-up appointments, which may lead to better health outcomes for the patient in the long term. Further, the amount of rewards awarded for the patient completing each action can be set and weighted towards task completions that result in the highest correlation with engagement, compliance, outcomes, and patient satisfaction. These reward amounts may be set, for example, based on patient studies (e.g., trials, commercial pilots, or equivalent) to determine the correlation between the completion of tasks or activities and patient outcomes (e.g., mortality, quality of life, recovery time, etc.) to determine the appropriate weighting for each category of tasks/activities, as well as the level of achievement within each category, to make beneficial health impacts for patients. Milestones may also be set for patients (e.g., for total rewards accrued) to encourage patients to repeat the same beneficial actions that will result in the best patient outcomes. As such, this gamification can be leveraged to encourage digital engagement by patients with a patient incentive rewards program, which in turn rewards behavior change and motivates patients to continue usage of prescribed devices and tasks/activities.

Further, as described above, the patient may be able to view their rewards progress on a digital engagement dashboard. The patient may also be able to view their rewards progress compared to other patients in a predetermined cohort. Viewing their progress compared to other patients in the predetermined cohort (e.g., including patients who are concurrently prescribed wearable cardiac monitoring devices and/or participating in other health programs such as cardiac rehabilitation) may further help motivate patients to comply with their prescription and perform actions associated with improved patient outcomes as a sense of community belonging can be a key element in driving health-related behavior changes. Having this predetermined cohort as a community may motivate patients to “keep up” and/or “compete” to try to achieve the best results in the patient engagement incentive program. The progress of each patient toward earning rewards may also be tailored to the patient, such as setting milestones and/or the rewards the patient earns for completing certain actions according to each patient's ability to meet certain goals (e.g., setting step count milestones according to the mobility of each patient and the patient's tolerance for exercise). This tailored approach addresses the issue that more direct measures, such as step count, activity level, heart health, etc., will vary depending on the overall health status of each patient. As such, the competition between the patients may keep kept to safe and fair levels regardless of each patient's overall status and health. This may further help ensure patient compliance by preventing patients from feeling burned out with attempting to meet goals or earn rewards that may be too difficult for them.

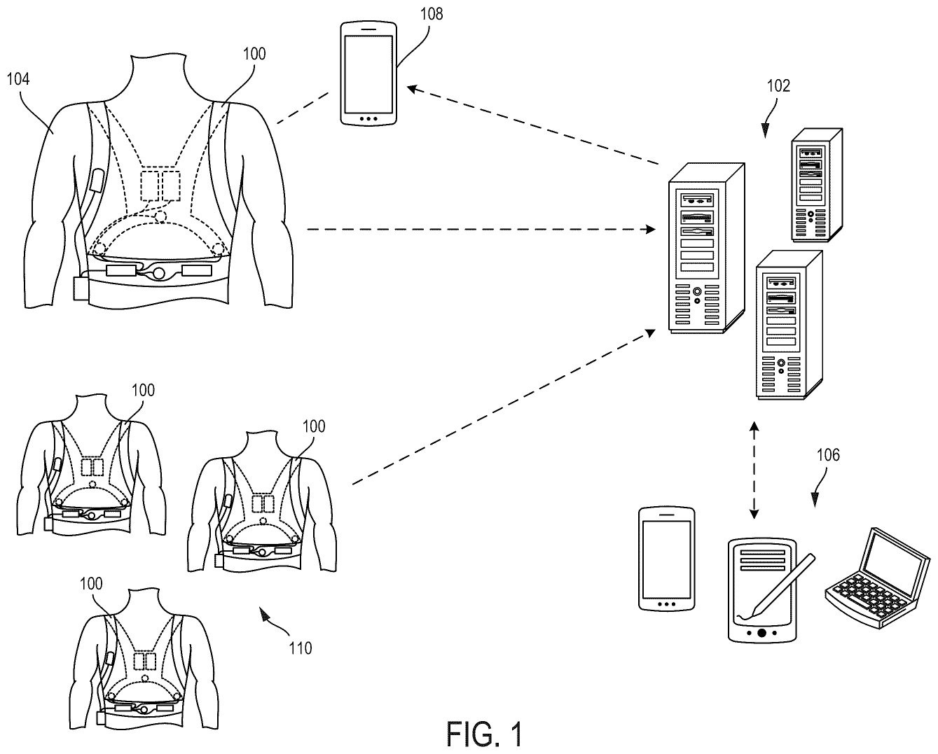

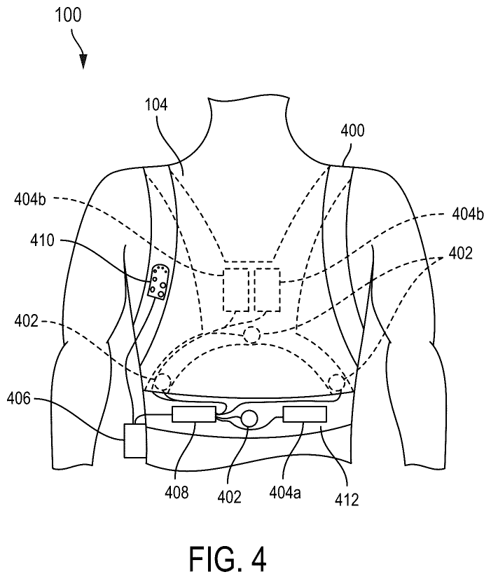

FIG. 1 shows a wearable cardiac monitoring and rehabilitative system that includes a wearable cardiac monitoring device 100 in communication with a remote server 102. The wearable cardiac monitoring device 100 is configured to continuously monitor and/or treat an ambulatory cardiac patient 104 for one or more arrhythmias. In implementations, the wearable cardiac monitoring device 100 may be implemented as a wearable garment configured to be worn continuously by the ambulatory cardiac patient 104 for an extended period of time. The wearable garment may include one or more electrocardiogram (ECG) sensing electrodes configured to output at least one ECG signal, one or more motion detectors configured to generate motion data indicative of physical activity performed by the ambulatory cardiac patient 104, and wear state detection circuitry configured to detect a wear state of the wearable cardiac monitoring device 100. In implementations, the wearable garment may further include one or more other externally worn sensors configured to output one or more other physiological signals for the ambulatory cardiac patient 104, such as one or more biovibrational sensors configured to output cardiac vibration signals, pulmonary vibration signals, or other biovibration signals. Additionally, in some implementations, the wearable garment may include one or more therapy electrodes. Accordingly, if the wearable cardiac monitoring device 100 detects a treatable arrhythmia, the wearable cardiac monitoring device 100 may provide a defibrillation shock or a cardioversion/defibrillation shock to the ambulatory cardiac patient 104. The wearable cardiac monitoring device 100 is described in further detail below with reference to FIGS. 4-11.

The wearable cardiac monitoring device 100 is configured to transmit signals and data generated by the wearable cardiac monitoring device 100 and transmit the signals and data to the remote server 102. Accordingly, the wearable cardiac monitoring device 100 may be in wireless communication with the remote server 102. As an illustration, the wearable cardiac monitoring device 100 may communicate with the remote server 102 via cellular networks, via Bluetooth®-to-TCP/IP access point communication, via Wi-Fi, and the like. As such, the wearable cardiac monitoring device 100 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the communications circuitry in the wearable cardiac monitoring device 100 may be part of an Internet of Things (IoT) and communicate with the remote server 102 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

The remote server 102 is configured to receive and process the signals and data transmitted by the wearable cardiac monitoring device 100 worn by the ambulatory cardiac patient 104. Accordingly, the remote server 102 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the signals transmitted by the wearable cardiac monitoring device 100. For example, the at least one processor of the remote server 102 may be configured similarly to the processor 516 of the wearable cardiac monitoring device 100 discussed in further detail below. The database may be implemented as flash memory, solid state memory, magnetic memory, optical memory, cache memory, combinations thereof, and/or others.

Additionally, as further shown in FIG. 1, the remote server 102 is configured to receive and process signals and data transmitted by additional wearable cardiac monitoring devices 100 worn by other ambulatory cardiac patients 110. In various implementations, the remote server 102 may use the signals and data transmitted by the wearable cardiac monitoring devices 100 to manage patient engagement incentives for ambulatory cardiac patients being monitored for cardiac arrhythmias (e.g., including the ambulatory cardiac patient 104 and the other ambulatory cardiac patients 110), as described in further detail below.

As shown in FIG. 1, the wearable cardiac monitoring and rehabilitative system further includes one or more user interfaces, such one or more clinician-authorized user terminals 106 and one or more patient user devices 108. The clinician-authorized user terminals 106 and patient user device 108 are in electronic communication with the remote server 102 through a wired or wireless connection. For instance, the clinician-authorized user terminals 106 and patient user device 108 may communicate with the remote server 102 via Wi-Fi, via Ethernet, via cellular networks, and/or the like. The clinician-authorized user terminals 106 and patient user device 108 may include, for example, desktop computers, laptop computers, and/or portable personal digital assistants (e.g., smartphones, tablet computers, etc.).

The one or more clinician-authorized user terminals 106 are configured to electronically communicate with the remote server 102 for the purpose of sending and receiving information relating to patients wearing wearable cardiac monitoring devices, such as the ambulatory cardiac patient 104 wearing the wearable cardiac monitoring device 100. In implementations, the clinician-authorized user terminals 106 are configured to receive incentive criteria for a predetermined patient engagement incentive program from a clinician and transmit the incentive criteria to the remote server 102. Examples of incentive criteria and patient engagement incentive programs are described in further detail below. Further, the user terminals 106 may be configured to allow clinicians to view information on patients wearing wearable cardiac monitoring devices, such as the ambulatory cardiac patient 104 wearing the wearable cardiac monitoring device 100. In some implementations, a clinician-authorized user terminal 106 may display to the user (e.g., a clinician or other caregiver associated with the ambulatory cardiac patient 104) one or more reports summarizing arrhythmia information for the patient 104, health information for the patient 104 (e.g., activity information for the patient 104, the average heart rate of the patient 104, how many hours per night the patient 104 has been sleeping, etc.), wear status information for the patient 104 (e.g., how many hours per day the patient 104 has been wearing the wearable cardiac monitoring device 100), and/or the like.

The one or more patient user devices 108 are associated with the ambulatory cardiac patient 104, as shown in FIG. 1. For example, a patient user device 108 may be a patient's personal smartphone, laptop computer, desktop computer, tablet computing device, and/or the like. As another example, a patient user device 108 may be a computing device specifically associated with the wearable cardiac monitoring and rehabilitative system. For instance, the patient user device 108 may be implemented in a portable gateway 604 clinician-aut, described in further detail below. Additionally, the one or more patient user devices 108 are configured to electronically communicate with the remote server 102 for the purpose of sending and receiving information relating to the predetermined patient engagement incentive program. For example, the patient user device 108 may receive data from the remote server 102 indicating complete or partial incentive rewards, offered as part of the patient engagement incentive program, that the patient 104 has earned. The patient user device 108 may then display these rewards to the patient 104. As another example, the patient user device 108 may receive data from the remote server 102 indicating the progress of the patient 104 towards earning incentive rewards relative to the progress of other patients wearing other wearable cardiac monitoring devices 100 and/or other patients in cardiac rehabilitation programs towards earning incentive rewards. For instance, the patient user device 108 may receive data from the remote server 102 indicating the progress of the patient 104 towards earning incentive rewards compared to the progress of each of the other ambulatory cardiac patients 110, or a subset of the other ambulatory cardiac patients 110, towards earning the same incentive rewards. The patient user device 108 may then display the progress of the patient 104 relative to the progress of the other ambulatory cardiac patients 110.

In some implementations, a clinician-authorized user terminal 106 and/or a patient user device 108 may be a specialized user interface configured to communicate with the remote server 102. As an example, the clinician-authorized user terminal 106 may be a specialized computing device configured to communicate with the remote server 102 to send and receive information relating to patients wearing wearable cardiac monitoring devices 100. As another example, the patient user device 108 may be a specialized computing device configured to receive information relating to a predetermined patient engagement incentive program, as well as display earned rewards and the progress of the patient 104 relative to other patients wearing other wearable cardiac monitoring devices 100.

In some implementations, a clinician-authorized user terminal 106 and/or a patient user device 108 may be a generalized user interface that has been adapted to communicate with the remote server 102. To illustrate, the clinician-authorized user terminal 106 may be a computing device (e.g., a laptop, a portable personal digital assistant such as a smartphone or tablet, etc.) executing a clinician application that configures the computing device to communicate with the remote server 102. For example, the clinician application may be downloaded from an application store or otherwise installed on the computing device. Accordingly, when the computing device executes the clinician application, the computing device is configured to establish an electronic communication link with the remote server 102 to receive and transmit information regarding patient wearing wearable cardiac monitoring devices 100. Similarly, the patient user device 108 may be a computing device (e.g., a laptop, a portable personal digital assistant such as a smartphone or tablet, etc.) executing a patient application that configures the computing device to communicate with the remote server 102. The patient application may be similarly downloaded from an application store or otherwise installed on the computing device and, when executed, may configure the computing device to establish a communication link with the remote server 102 to receive and display information on a predetermined patient engagement incentive program.

The application store is typically included within an operating system of a computing device implementing a user interface. For example, in a device implementing an operating system provided by Apple Inc. (Cupertino, California), the application store can be the App Store, a digital distribution platform, developed and maintained by Apple Inc., for mobile apps on its iOS and iPadOS® operating systems. The application store allows a user to browse and download an application, such as the clinician or patient application, developed in accordance with the Apple® iOS Software Development Kit. For instance, such technician or caregiver application may be downloaded on an iPhone® smartphone, an iPod Touch® handheld computer, or an iPad® tablet computer, or transferred to an Apple Watch® smartwatch. Other application stores may alternatively be used for other types of computing devices, such as computing devices operating on the Android® operating system.

In some implementations, the clinician application and the patient application may be the same application, and the application may provide different functionalities to the computing device executing the application based on, for example, credentials provided by the user. For instance, the application may provide clinician functionalities to a first computing device in response to authenticating clinician credentials entered on the first computing device, and may provide patient functionalities to a second computing device in response to authenticating patient credentials entered on the second computing device. In other cases, the clinician application and the patient application may be separate applications, each providing separate functionalities to a computing device executing them.

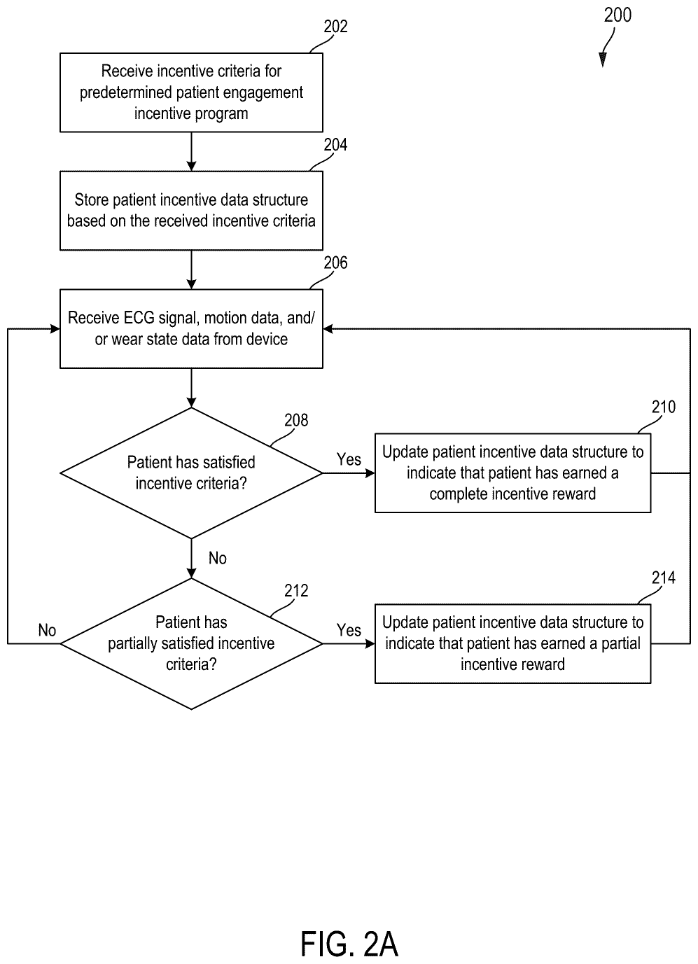

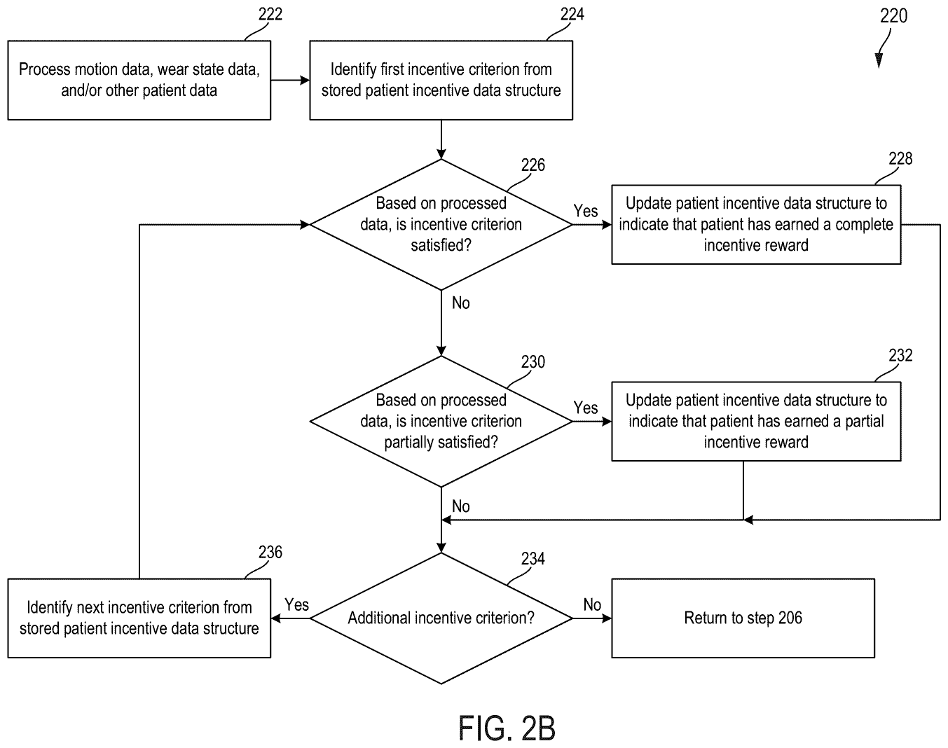

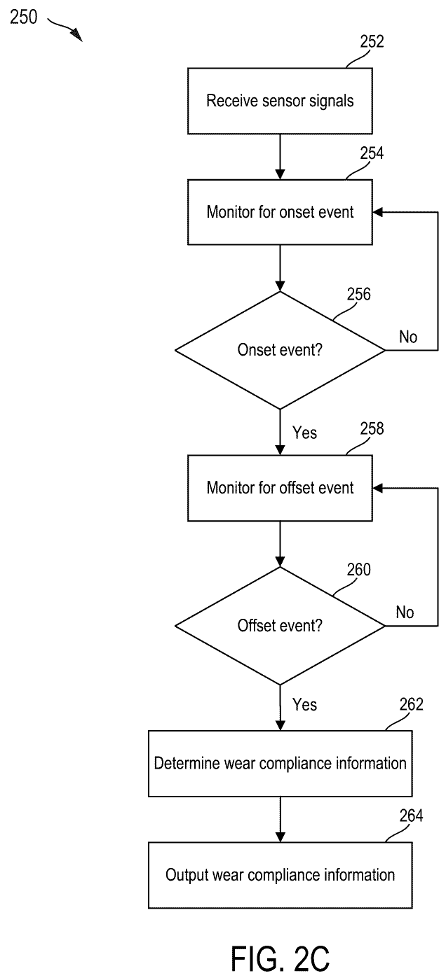

FIG. 2A illustrates a sample process flow for managing patient engagement incentives for ambulatory cardiac patients being monitored for cardiac arrhythmias. The sample process 200 shown in FIG. 2A can be implemented by a remote server 102. As shown in FIG. 2A, the remote server 102 receives incentive criteria at step 202. In implementations, the incentive criteria are for a predetermined patient engagement program for the ambulatory cardiac patient 104. As such, the remote server 102 may receive the incentive criteria from a clinician, for example, via a clinician-authorized user terminal 106.

The incentive criteria may define goals for the patient 104 to achieve in order to earn a complete and/or partial reward. Rewards may include one or more types of predetermined rewards indicia (e.g., virtual rewards), such as points, ribbons, badges, emotes, avatars, and/or the like. The virtual rewards may be standalone rewards, or the patient 104 may be able to exchange or redeem the virtual rewards for other types of prizes. For example, the patient 104 may be able to redeem points for gift cards, items from a catalogue, donations to a charity, etc.

As an example, the incentive criteria may include a physical activity goal for the patient 104, such as a number of steps for the patient 104 to perform, a number of minutes of physical activity for the patient 104 to perform, and/or a predetermined combination of the two. As another example, the incentive criteria may include an amount of time for the patient 104 to wear the wearable cardiac monitoring device 100, such an amount of time per day, per week, etc. (e.g., measured in hours, measured in percentage of the day, etc.) for the patient 104 to wear the wearable cardiac monitoring device 100. As another example, the incentive criteria may include a health goal for the patient 104, such as a resting heart goal, an R-R interval goal, a heart rate variability goal, and/or a health improvement goal. A health improvement goal may, for instance, be a number or percentage improvement over a starting health value. To illustrate, a health improvement goal may be a 3 bmp, 5 bpm, 8 bpm, 10 bbpm, etc. improvement over a baseline heart rate determined when the patient 104 is fitted for or receives the wearable cardiac monitoring device 100. A health improvement goal may be a 50 ms, 100 ms, 200 ms, 300 ms, 400 ms, 500 ms, etc. improvement over a baseline R-R interval. A health improvement goal may be a 3 ms, 5 ms, 8 ms, 10 ms, etc. improvement over a baseline heart rate variability value. A health improvement goal may be a 2%, 3%, 5%, 8%, 10%, 15%, 20%, etc. improvement over a starting health value, such as a baseline heart rate, baseline R-R interval, or baseline heart rate variability value. As another example, the incentive criteria may include a sleep goal for the patient. For instance, the clinician may set a goal that the patient 104 get at least a certain number of hours of sleep per night (e.g., at least 7 hours of sleep, at least 8 hours of sleep, etc.).

In some implementations, the incentive criteria may include an action that the patient 104 needs to perform. In examples, the incentive criteria may include the patient 104 acknowledging a message sent to the patient 104 (e.g., by the remote server 102 or another messaging system). As an example, the patient 104 may receive a message related to the prescription of the patient 104 to wear the wearable cardiac monitoring device 100. For instance, the message may contain instructions for how to assemble, wear, and disassemble the wearable cardiac monitoring device 100; instructions for caring for the wearable cardiac monitoring device 100; information about the duration of the prescription of the patient 104; information about a health improvement plan prescribed by a clinician of the patient 104; tips for improving the cardiac health of the patient 104; and/or so on. The message may include text, pictures, video, audio, etc. The patient 104 may receive this message as a text or SMS message (e.g., texted to the patient user device 108), as an email, within an application related to the wearable cardiac monitoring device 100 that is running on the patient user device 108, and/or the like. As such, in examples, acknowledging the message as part of the incentive criteria may include the patient 104 opening the message. In examples, the incentive criteria may also include the patient 104 opening messages on an ongoing basis, such as the requiring the patient 104 to open weekly messages sent to the patient 104 about their cardiac health and/or the amount of time the patient 104 has been wearing the wearable cardiac monitoring device 100. In examples, acknowledging the message as part of the incentive criteria may include the patient 104 clicking or otherwise interacting with the content of the message. To illustrate, a text or SMS message may include a link to a landing page associated with the wearable cardiac monitoring and rehabilitative system, a link to review chapters of a patient education video, etc. Accordingly, acknowledging the message may include clicking on the link in the message. In implementations, a copy of the message or a copy of the content of the message sent to the patient 104 may also be sent to the patient's clinician.

As another example, the patient 104 may receive a video sent to the patient 104. To illustrate, the video may show the patient 104 how to assemble the wearable cardiac monitoring device 100, how to disassemble the wearable cardiac monitoring device 100, how to put on and take off the wearable cardiac monitoring device 100, how to take out and charge a battery of the wearable cardiac monitoring device 100, what happens when the wearable cardiac monitoring device 100 detects a treatable arrhythmia, how to avoid a defibrillation shock if the wearable cardiac monitoring device 100 detects a treatable arrhythmia and the patient 104 is still conscious, and/or so on. As with the message described above, the patient 104 may receive the video as a text or SMS message, as an email, within an application running on the patient user device 108, and/or the like. Accordingly, acknowledging the message as part of the incentive criteria may include the patient 104 watching the video.

As another example, the patient 104 may receive a questionnaire sent to the patient 104. The questionnaire may be sent periodically (e.g., daily, weekly, monthly, etc.), and/or the questionnaire may be sent in response to a certain event (e.g., the patient 104 meeting a health goal, a detected equipment malfunction with the wearable cardiac monitoring device 100, the patient 104 experiencing and being treated for a treatable cardiac arrhythmia, etc.). The questionnaire may ask the patient 104 about their experiences with wearing the wearable cardiac monitoring device 100, about any cardiac-related symptoms the patient 104 has been experiencing (e.g., chest pain, shortness of breath, fatigue, trouble sleeping without multiple pillows, etc.) and when the patient 104 has experienced those symptoms, how often and for how long the patient 104 wears the wearable cardiac monitoring device 100, and/or so on. As with the message described above, the patient 104 may receive the questionnaire as a text or SMS message, as an email, within an application running on the patient user device 108, and/or the like. Accordingly, acknowledging the message as part of the incentive criteria may include the patient completing the questionnaire.

In examples, the incentive criteria may include the patient 104 attending an appointment with a caregiver. For instance, a clinician may transmit the time, date, and location of one or more appointments that the patient 104 has with the clinician and/or with other caregivers. The clinician may enter the appointment(s) manually, or the remote server 102 may automatically receive appointment information for the patient 104 (e.g., by interfacing with an appointment system used by the clinician, by receiving appointment information from the patient user device 108, etc.).

In examples, the incentive criteria may include the patient 104 sharing information related to a health status of the patient 104 and/or a status of the wearable cardiac monitoring device 100 with a caregiver. As an example, the patient 104 may share information with the clinician who sent the incentive criteria or another clinician associated with the patient 104 and/or share information with a friend or family member helping to care for the patient 104 (e.g., if the patient 104 is requested to share health status information). As another example, the patient 104 may share information with a manufacturer of the wearable cardiac monitoring device 100 (e.g., if the patient is requested to share the status of the wearable cardiac monitoring device 100). The patient 104 may share information via an application running on the patient user device 108, via an email, via a phone call, via a text or SMS message, by filling out a form on a website associated with the clinician and/or the wearable cardiac monitoring device 100, and/or the like.

In implementations, the clinician may provide the incentive criteria to the remote server 102 by manually selecting incentive criteria for the patient 104. For example, the remote server 102 may provide a list of possible incentive criteria to the clinician-authorized user terminal 106, and the clinician may select incentive criteria for the patient 104 from the list. The list of possible incentive criteria may be standardized to all patients, or the list of possible incentive criteria may be individualized to the patient 104. For instance, the clinician may input demographic information, health information (e.g., a classification of the health of the patient 104, such as the heart failure stage of the patient 104 according to the American Heart Association (AHA) or the New York Heart Association (NYHA)), etc. The remote server 102 may then provide a list of possible incentive criteria to the clinician according to, for example, the demographics and/or predetermined classification of the health of the patient 104. As another example, the remote server 102 may provide a list of standard incentive criteria to the clinician-authorized user terminal 106, where each of the standard incentive criteria can be customized to the patient 104 and the desired outcomes for the patient 104 based on information input by the clinician. The clinician may then input information for the patient 104 for each of the standard incentive criteria. To illustrate, one standard incentive criterion may be to reduce the resting heart rate for the patient 104. Accordingly, the clinician may input the current resting heart rate for the patient 104, a desired resting heart rate for the patient 104, and a time period for the patient 104 to achieve the resting heart rate. As another illustration, a standard incentive criterion may be for the patient 104 to perform a certain amount of physical activity per day and/or per week. The clinician may thus enter in the desired amount of physical activity for the patient 104 to perform per day and/or per week.

In implementations, the clinician may provide the incentive criteria to the remote server 102 by giving the remote server 102 information about the patient 104, and the remote server 102 may automatically or partially automatically select the incentive criteria for the patient 104 based on the provided information. As an example, the clinician may input the gender, height, weight, resting heart rate, heart failure stage (e.g., stage A-D as classified by the AHA or stage I-IV as classified by the NYHA), other demographic information, other health information, and/or other biometric information for the patient 104. The remote server 102 may then automatically select incentive criteria for the patient 104 using the information for the patient 104 provided by the clinician. For instance, the remote server 102 may determine where the patient 104 fits into one or more demographic categories based on the provided information, where each demographic category has one or more associated incentive criteria. As such, the remote server 102 may set the incentive criteria for the patient 104 based on the incentive criteria for the demographic category or categories that the patient 104 fits into. As another illustration, the remote server 102 may determine the incentive criteria for the patient 104 based on a predetermined classification of the health of the patient 104, such as based on a heart failure stage of the patient 104, based on the resting heart rate of the patient 104, based on the ejection fraction of the patient 104, and/or based on a combination of health factors (e.g., a combination of the patient's age, resting heart rate, activity level, and/or the like). Each health classification may have an associated list of one or more incentive criteria, and the remote server 102 may set the incentive criteria for the patient 104 based on the predetermined classification of the health of the patient 104.

As another example, the clinician may provide desired outcomes for the patient 104, and the remote server 102 may automatically set the incentive criteria for the patient 104 based on the desired outcomes. To illustrate, the clinician may provide desired health outcomes for the patient 104, such as a desired resting heart rate, desired amount of physical activity per day and/or per week, a desired R-R interval, a desired heart rate variability, and/or the like for the patient 104. As another illustration, the clinician may provide desired patient actions to the remote server 102, such as the patient 104 making and keeping a certain frequency of appointments with the clinician, the patient 104 wearing the wearable cardiac monitoring device 100 for a certain amount of time each day, the patient 104 watching certain informational videos about how to improve the health of the patient 104 and/or how to use and maintain the wearable cardiac monitoring device 100, and/or the like. Based on the desired outcomes provided by the clinician, the remote server 102 may determine incentive criteria that will help the patient 104 meet the desired outcomes. For instance, if the clinician has input a desired resting heart rate for the patient 104, the remote server 102 may determine an amount of physical activity the patient 104 should engage in every day and/or every week to help lower the current resting heart rate of the patient 104 over time.

In implementations, the clinician and/or the remote server 102 may determine a predetermined time period during which the patient 104 must complete one or more actions as part of receiving and setting the incentive criteria. The predetermined time period may be different for each incentive criterion and, for example, may be a day, 2 days, 3 days, 5 days, a week, a month, 3 months, or 6 months. In examples, the predetermined time period may be user-configurable (e.g., by the clinician). For example, the clinician may set an amount of time that the patient 104 should engage in physical activity per day and/or per week. The incentive criterion may start at a default setting, such as 30 minutes per day or 150 minutes per week, and the clinician may be able to adjust the amount of physical activity per day and/or per week based on the patient 104. To illustrate, the clinician may increase the amount of physical activity for a mobile patient 104 capable of carrying out more physical activity without suffering from cardiac distress and may decrease the amount of physical activity for a less mobile patient 104 and/or a patient capable of less physical activity without suffering from cardiac distress.

In implementations, at least some of the incentive criteria may be individualized or personalized to the patient, such as individualized to the patient based on the demographics and/or a predetermined health classification for the patient 104 as described above. In implementations, at least some of the incentive criteria may be standardized to all patients wearing a wearable cardiac monitoring device 100, such as the ambulatory cardiac patient 104 and the other ambulatory cardiac patients 110. For example, the incentive criteria for all patients may include a criterion that the patient watch informational videos on how to assemble, wear, disassemble, and clean the wearable cardiac monitoring device 100. As another example, the incentive criteria for all patients may include a criterion that the patient wear the wearable cardiac monitoring device for a predetermined amount of time each day.

In implementations, the incentive criteria may include multiple rewards thresholds. For example, incentive criteria may include a first threshold that the patient 104 can meet to earn a partial reward and a second threshold that the patient 104 can meet to earn a complete or full reward. To illustrate, the incentive criteria may include a criterion that that patient should exercise 30 minutes per day to earn a complete reward and 20 minutes per day to earn a partial reward. As another illustration, the incentive criteria may include a criterion that the patient should watch 10 informational videos to earn a complete reward and 5 informational videos to earn a partial reward. As another illustration, the incentive criteria may include a criterion that the patient earns a partial reward when the patient 104 decreases their resting heart rate by 3 bpm and earns a complete reward when the patient 104 decreases their resting heart rate by 6 bpm.

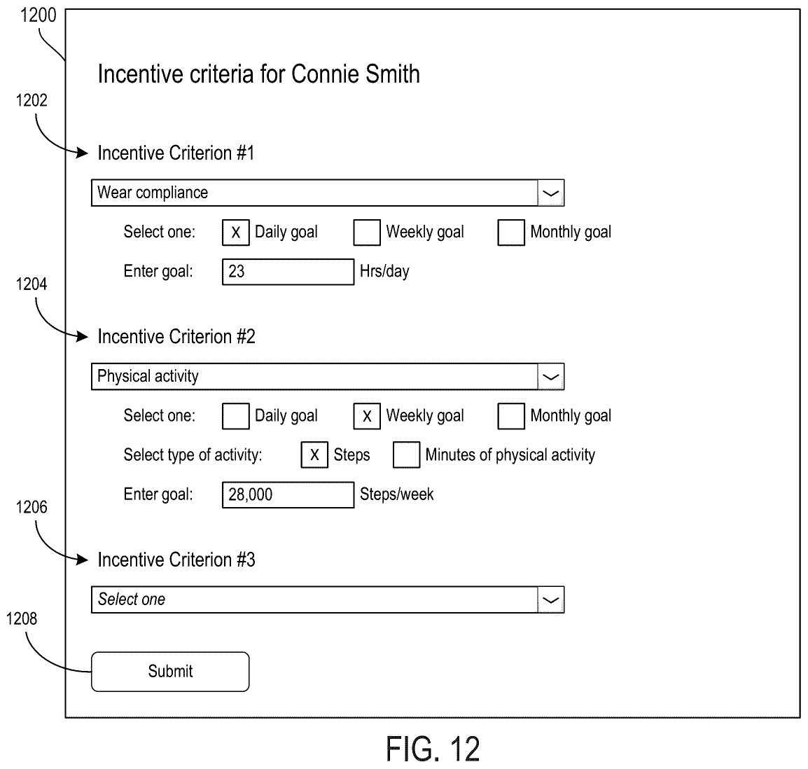

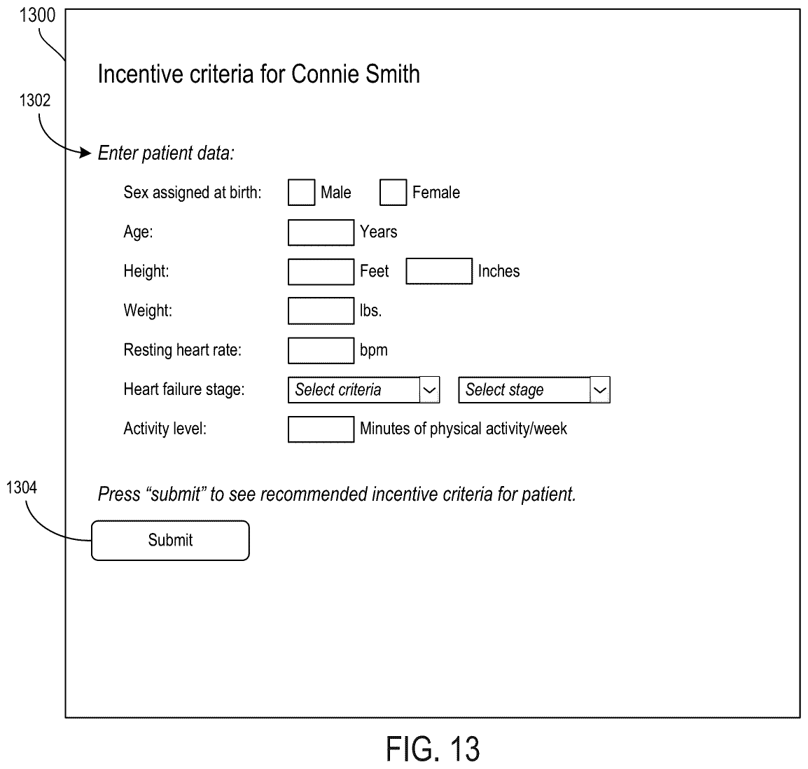

As examples, FIGS. 12 and 13 depict user interfaces for entering patient incentive criteria according to some embodiments. The user interface 1200 of FIG. 12 and the user interface 1300 of FIG. 13 may be displayed, for example, on clinician-authorized user terminals 106. Referring first to FIG. 12, the user interface 1200 shows an illustration of how incentive criteria may be gathered for an example patient 104, in this case “Connie Smith.” The user interface 1200 includes sections 1202, 1204, and 1206 configured to receive incentive criteria for the patient 104 from a clinician for the patient 104. As shown in FIG. 12, the clinician has selected “wear compliance” from a drop-down menu in section 1202. From the selection of wear compliance from the drop-down menu, the user interface 1200 has populated options for the clinician to fill in within section 1202, specifically, for the clinician to select a frequency of the goal (e.g., a daily goal, weekly goal, or monthly goal) and an amount for the goal (e.g., in hours/day, hours/week, or hours/month). In the example user interface 1200, the clinician has selected a daily goal of 23 hours/day. In embodiments, by selecting wear compliance, the clinician may be presented with a default wear compliance goal that the clinician can modify, such as a default wear compliance goal of 22 hours/day that the clinician can modify to 23 hours/day as shown.

In section 1204, the clinician has selected “physical activity” from a drop-down menu. From the selection of physical activity, the user interface 1200 has also populated options for the clinician to fill in within section 1204, specifically, for the clinician to select a frequency of the goal (e.g., a daily goal, weekly goal, or monthly goal), a type of physical activity (e.g., steps or minutes of physical activity), and an amount for the goal (e.g., in steps/day, steps/week, steps/month, minutes of physical activity/day, minutes of physical activity/week, or minutes of physical activity/month). As shown in FIG. 12, in the example user interface 1200, the clinician has selected a weekly goal of 28,000 steps/week. Similar to the description of the section 1202 above, in embodiments, by selecting physical activity, the clinician may be presented with a default physical activity goal that the clinician can modify (e.g., 5,000 steps/day).

As further illustrated in FIG. 12, the user interface 1200 includes an additional section 1206 that the clinician has not or not yet selected an incentive criterion for the patient 104 in. Examples of other incentive criteria the clinician may select from using the drop-down menu of section 1206 may include hours of sleep per night, a target heart rate to maintain, a target R-R interval to maintain, a health improvement goal, an appointment goal, an informational video goal, and/or the like. In embodiments, if the clinician selects an incentive criterion for the patient 104 using section 1206, the user interface 1200 may repopulate to include an additional section by which the clinician can select a fourth incentive criterion, and so on. In embodiments, there may be a limit to the number of incentive criteria the clinician may select for the patient 104 (e.g., a limit of 5, 8, 10, 12, 15, etc.).

At the bottom of the user interface 1200 is a “submit” button 1208. The clinician may press the submit button 1208 to transmit the incentive criteria to the remote server 102. In embodiments, the incentive criteria for the patient 104 thus includes the incentive criteria selected for the patient 104 using the user interface 1200. In embodiments, the incentive criteria for the patient 104 may include the incentive criteria selected for the patient 104 using the user interface 1200 as well as one or more default incentive criteria. As an illustration, in addition to the wear compliance and physical activity incentive criteria for the patient 104, the patient's incentive criteria may include a default incentive criterion to watch a series of educational videos on how to use and care for the wearable cardiac monitoring device 100.

In embodiments, the clinician may be able to edit the incentive criteria for the patient 104 after submitting the incentive criteria to the remote server 102. For example, the clinician may be presented with a confirmation screen that shows the incentive criteria the clinician has selected for the patient 104 that the clinician must confirm before the clinician-authorized user terminal 106 submits the incentive criteria to the remote server 102. As another example, the clinician may be able to access a clinician portal associated with the predetermined patient engagement program, view the incentive criteria for the patient 104 within the portal, and use the portal to modify the incentive criteria.

Referring next to FIG. 13, the user interface 1300 shows another illustration of how incentive criteria may be gathered for an example patient 104, again named “Connie Smith.” The user interface 1300 includes a section 1302 whereby the clinician can enter patient data for the patient 104. The patient data may, for instance, include demographic data, health data, biometric data, and/or the like. For example, as shown in FIG. 13, the section 1302 includes fields for “sex assigned at birth,” “age,” “height,” “weight,” “resting heart rate,” “heart failure stage” (e.g., for the clinician to select from the AHA or NYHA criteria and a stage of the AHA or NYHA criteria), and “activity level” (e.g., in estimated minutes of physical activity per week). The user interface 1300 further includes a “submit” button 1304 that the clinician may press to see recommended incentive criteria for the patient 104 determined from the patient data input in section 1302. In embodiments, the clinician may modify the recommended incentive criteria, such as by adding one or more incentive criteria, deleting one or more incentive criteria, and/or changing the parameters of one or more incentive criteria.

The remote server 102 stores a patient incentive data structure based on the received incentive criteria at step 204. As an illustration, in implementations, the remote server 102 may set a series of functions and/or variables for the patient 104 based on the received incentive criteria. For example, the remote server 102 may create a new data structure for the patient 104 upon receiving the incentive criteria. If the incentive criteria include a criterion that the patient engage in physical activity or other exercise for 150 minutes per week, the remote server 102 may store an entry in the data structure indicating that the remote server 102 should check the patient's physical activity (e.g., by storing a call function for determining physical activity in the data structure). The remote server 102 may also store a variable indicating that the threshold for the patient earning a reward is 150 minutes of physical activity per week (e.g., by setting a variable to 150 and storing the variable in the data structure). If the incentive criteria include a criterion that the patient achieve a resting heart rate of 75, the remote server 102 may store an entry in the data structure indicating that the remote server 102 should check the patient's heart rate (e.g., by storing a call function for determining heart rate in the data structure), as well as a variable indicating that the threshold for the patient earning a reward is a heart rate of 75 (e.g., by setting a variable to 75 and storing the variable in the data structure).

In implementations, the remote server 102 may also store the reward(s) that the patient earns for completing the incentive criteria in the patient incentive data structure. The clinician may determine and set the rewards that the patient 104 earns for completing an incentive criterion, and/or the remote server 102 may determine and set the rewards that the patient 104 earns for completing an incentive criterion. For example, each incentive criterion may be associated with a default reward. As such, the remote server 102 may set the reward for each incentive criterion to be the default reward. The remote server 102 may accordingly set the reward by storing the default reward in the patient incentive data structure (e.g., by setting a variable to be the default number of points the patient 104 earns by completing the incentive criterion, by setting a pointer to the default reward as stored in another data structure, etc.). Otherwise, the default reward may be incorporated as part of a function used to determine whether the patient 104 has earned a complete and/or partial reward.

As another example, the clinician may set the reward for each incentive criterion when the clinician provides the incentive criterion to the remote server 102 at step 202 (e.g., by manually entering the reward for the incentive criterion, by selecting the reward for the incentive criterion from a list of possible rewards, etc.). As another example, the remote server 102 may set the reward for each incentive criterion to be a default reward, but the clinician can raise or lower the level of the reward (e.g., when the clinician provides the incentive criteria to the remote server 102). In examples where the reward is individualized or customized to the patient 104, the remote server 102 may set the reward by storing the reward in the patient incentive data structure. For instance, if the clinician has specified a number of points that the patient 104 earns for completing an incentive criterion, the remote server 102 may store a variable set to that number of points in the patient incentive data structure. As a further illustration, if the clinician has selected a rewards level for an incentive criterion from a range of possible rewards levels for the criterion, the remote server 102 may store a variable set to the selected level in the patient incentive data structure, or the remote server 102 may store a pointer indicating the selected level as stored in another data structure.

In implementations, the remote server 102 may assign one or more predetermined weights for the incentive criteria and store the assigned predetermined weight(s) in the patient incentive data structure. For example, a weight for an incentive criterion may be input by the clinician (e.g., when the clinician provides the incentive criterion to the remote server 102). As another example, a weight for an incentive criterion may be a default weight for the criterion. For instance, an incentive criterion to engage in a certain level of physical activity per week may be at a default first predetermined weight, and an incentive criterion to fill out a patient survey may be at a default second predetermined weight that is lower than the default first predetermined weight.

As another example, each incentive criterion may have a default weight, but the clinician may be able to modify the default weight. To illustrate, an incentive criterion to wear the wearable cardiac monitoring device 100 for at least 20 hours every day may have a default weight of 3 on a scale of 1-5. However, the clinician may observe (e.g., on a clinician dashboard or portal presented to the clinician via the clinician-authorized user terminal 106) that the patient has been consistently wearing the wearable cardiac monitoring device 100 for about 15 hours every day. As such, the clinician may increase the weight for the incentive criterion to wear the wearable cardiac monitoring device 100 for at least 20 hours every day to 4.

As another example, the remote server 102 may automatically determine the weight for each incentive criterion based on information about the patient and/or the wearable cardiac monitoring device 100. For instance, the clinician may provide current health information and desired health outcomes for the patient 104 to the remote server 102, and the remote server 102 may use the desired health outcomes compared to the current health information to determine weights for the incentive criteria. As an illustration, the incentive criteria may include the following goals for the patient: (a) fill out a weekly survey on the health and status of the patient 104, (b) wear the wearable cardiac monitoring device 100 for at least 22 hours per day, (c) engage in at least 150 minutes of physical activity per week, and (d) maintain a resting heart rate of 65-75 bpm. The clinician may also provide health information to the remote server 102 indicating that the patient reports comes in for regular appointments, is exercising less than an hour each week, and has a resting heart rate of 71. As the patient exercises less than 60 minutes each week, the remote server 102 may determine that (c) engaging in at least 150 minutes of physical activity per week should receive the highest weighting (e.g., weighting (c) at an 8 out of possible 10). Because the patient comes in for regular appointments, the remote server 102 may weight (a) filling out a weekly survey relatively low (e.g., weighting (a) at a 2). Because the resting heart rate of the patient 104 is already at 71, the remote server 102 may set the weight for (d) maintaining a resting heart rate of 65-75 bpm at a medium level (e.g., weighting (d) at a 5).

In implementations, the weight assigned to each incentive criterion is associated with a risk score for the health of the patient 104 if the patient 104 does not satisfy the incentive criterion. For instance, referring back to the previous example, the remote server 102 may weight (a) filling out a weekly survey at a 2 partially because not filling out the weekly survey has a relatively low risk of adversely impacting the health of the patient 104. However, the remote server 102 may weight (d) maintaining a resting heart rate of 70 at a medium level, even though the patient 104 is already meeting this incentive criterion, because maintaining a lower resting heart rate is associated with better patient outcomes. Additionally, the remote server 102 may weight (b) wearing the wearable cardiac monitoring device 100 for at least 22 hours a day relatively high because if the patient experiences a treatable cardiac arrhythmia while not wearing the device 100, the patient 104 could potentially die (e.g., weighting (b) at an 8). The risk scores for the incentive criteria may be based on statistics determined for patient populations (e.g., as determined in clinical studies), based on past patient outcomes, based on known risk factors for the demographic populations that the patient 104 belongs to, and/or the like.

In implementations where incentive criteria are assigned weights, the remote server 102 may, for example, store the weight for each incentive criterion in the patient incentive data structure (e.g., as a data entry in a matrix for the patient 104, as a variable set to the weight, as a pointer indicating the weight as stored in a separate data structure, etc.). In implementations, the weights may be associated with different rewards sizes and/or types. For instance, each incentive criterion may be weighted on a scale of 1 to 5, 1 to 10, or the like. The number of points or ribbons the patient 104 earns for completing the incentive criterion may be a function of the maximum amount of points for that type of incentive criterion and the weight for the incentive criterion. As an example, an incentive criterion to click on a text message sent to the patient 104 may be weighted at a 1 out of 5, and the maximum number of points possible for clicking on a text message may be 2.5. Accordingly, in this example, the rewards that the patient 104 earns by clicking on a text message sent to the patient 104 is 0.5 points. As another example, an incentive criterion to exercise for 30 minutes per day may be weighted at a 3 out of 5, and the maximum number of points possible for daily exercise may be 5, such that the rewards the patient 104 earns for each day the patient 104 exercises at least 30 minutes is 3 points. To illustrate another instance, each incentive criterion may be weighted on a scale of 1 to 5. However, for any incentive criteria weighted as a 4 or 5, the patient may receive a badge in addition to points. Thus, as an example, an incentive criterion to attend a follow-up appointment may be weighted as a 5, and the maximum rewards possible for attending an appointment is 10. Thus, if the patient attends a follow-up appointment, the patient 104 receives 10 points as well as a badge. In implementations, the remote server 102 may, instead of storing a weight for the incentive criterion, store the size and/or type of reward as predetermined by the weight in the patient incentive data structure (e.g., as a data entry in a matrix for the patient 104, as variable(s) set to the size and/or type of reward, as a pointer indicating the size and/or type of reward in a separate data structure, etc.). Illustrative examples of the data structure are described further below with reference to Tables 1-5 below.

The remote server 102 receives at least one ECG signal, motion data, and/or wear state data from the wearable cardiac monitoring device 100 over a period of time at step 206. For example, as described in further detail below, the wearable cardiac monitoring device 100 may include one or more ECG sensing electrodes configured to output at least one ECG signal, one or more motion detectors configured to generate motion data indicative of physical activity performed by the ambulatory cardiac patient, and wear state detection circuitry configured to detect wear state data of the wearable cardiac monitoring device 100. In implementations, the wear state data may include data indicating whether the wearable cardiac monitoring device 100 is powered on, user inputs from the patient 104 indicating whether the patient 104 is wearing the wearable cardiac monitoring device 100, the at least one ECG signal, the motion data, impedance data from a skin sensor (e.g., which transmits a falloff signal into the body of the patient 104 if the patient 104 is wearing the wearable cardiac monitoring device 100 and senses the falloff signal back from the body of the patient 104 to verify that the patient 104 is wearing the wearable cardiac monitoring device 100), and/or the like.

In implementations, the remote server 102 may also receive additional patient data for the patient 104 at step 202. In examples, the remote server 102 may receive message acknowledgement data for the ambulatory cardiac patient 104. For instance, if the patient 104 opens a message sent to the patient user device 108 (e.g., as a text or SMS message, within an application running on the patient user device 108, etc.), the patient user device 108 may generate and transmit an indication to the remote server 102 that the patient 104 has opened the message. As another illustration, if the patient 104 visits a landing page that the patient 104 has been requested to visit, the server hosting the splash page (e.g., the remote server 102 or another server system) may generate and send an indication to the remote server 102 that the patient 104 has visited the splash page. As another illustration, if the patient 104 watches an educational video within an application running on the patient user device 108, the patient user device 108 may generate and transmit an indication to the remote server 102 that the patient has viewed the video. As another illustration, if the patient 104 fills out a questionnaire (e.g., by visiting a website, on an application running on the patient user device 108, etc.), the patient user device 108, server hosting the questionnaire, etc. may generate and send an indication to the remote server 102 that the patient 104 has filled out the questionnaire.

In examples, the remote server 102 may receive appointment data for the ambulatory cardiac patient 104. As an illustration, the remote server 102 may receive data from the clinician (e.g., via the clinician-authorized user terminal 106) indicating when the patient 104 has attended an appointment. As another illustration, the remote server 102 may receive global positioning system (GPS) data from the wearable cardiac monitoring device 100, which the remote server 102 can use to determine when the patient 104 has attended an appointment (e.g., by determining when the patient 104 has been at the location of the patient's clinician for a threshold amount of time). The remote server 102 may also receive appointment information from the patient 104 to use in determining whether the patient 104 has attended an appointment, such as calendar entries for appointments (e.g., stored in a calendar application on the patient user device 108).

In examples, the remote server 102 may receive information sharing data related to the patient 104 sharing information about a health status of the ambulatory cardiac patient 104 and/or a status of the wearable cardiac monitoring device 100 with the clinician. To illustrate, the remote server 102 may receive an indication from the clinician (e.g., via the clinician-authorized user terminal 106) that the patient 104 has sent health status/device status information that the clinician needed from the patient 104. As another illustration, the remote server 102 may receive an indication that the patient 104 has filled in patient information about a health status of the patient 104 (e.g., a questionnaire about symptoms or biometrics, such as blood pressure, of the patient 104) and/or information about a status of the wearable cardiac monitoring device 100 via an application running on the patient user device 108. For instance, the remote server 102 may receive the information sharing data from the patient user device 108 and/or from a server communicating with the patient user device 108 (e.g., the remote server 102 or another server system).

Using the signals and data received at step 206, the remote server determines whether the patient 104 has satisfied the incentive criteria at step 208. As shown in FIG. 2A, if the patient 104 has satisfied the incentive criteria, the remote server 102 updates the patient incentive data structure to indicate that the patient 104 has earned a complete incentive reward at step 210 and returns to monitoring for transmissions of signals and data at step 206. If the patient 104 has not satisfied the incentive criteria, the remote server 102 determines whether the patient has partially satisfied the incentive criteria at step 212. If the patient 104 has partially satisfied the incentive criteria, the remote server 102 updates the patient incentive data structure to indicate that the patient 104 has earned a partial incentive reward at step 214 and returns to monitoring for transmissions of signals and data at step 206. If the patient 104 has not fully satisfied or partially satisfied the incentive criteria, the remote server 102 makes no updates to the patient incentive data structure and returns to monitoring for transmissions of signals and data at step 206.

FIG. 2B shows a sample process 220 further illustrating examples of implementing steps 208, 210, 212, and 214 of FIG. 2A. As shown in FIG. 2B, the remote server 102 processes the motion data, wear state data, and/or other patient data (e.g., received at step 206 of FIG. 2A) at step 222. In implementations, with respect to the motion data, the remote server 102 may process the motion data to determine when the patient 104 was moving, when the patient was at rest, when the patient 104 was physically active, what the posture of the patient 104 was, and/or the like. As an illustration, if the motion data was captured by a 3D accelerometer with three axes (e.g., having a sampling rate of 50 Hz and a dynamic range of 16 bit), the motion data may include posture information for the patient 104. More specifically, the posture may be calculated by measuring the projected earth gravity vector in each of the three axes of the accelerometer. The device tilt may thus be measured as the arcsine of the ratio between the measured acceleration and earth's gravitational factor (g). In some cases, this posture information determination may be made at the remote server 102, which receives, for example, the projected earth gravity vectors from a motion detector of the wearable cardiac monitoring device 100 that includes the 3D accelerometer. In other cases, this determination may be made by a motion detector of the wearable cardiac monitoring device 100, which transmits motion signals to the remote server 102 that include a device tilt signal that corresponds to posture information for the patient 104 over time.

For example, in some implementations, the wearable cardiac monitoring device 100 may include a tri-axial accelerometer sensitive to the gravitational field, where the accelerometer reading samples during the measurement time window may be represented as xᵢ, yᵢ, and zᵢ. For each axis, the remote server 102 may normalize the accelerometer samples by the g (gravity) value. The remote server 102 may then average the accelerometer measurements over the measurement time window, which may be represented as:

xₐ=mean(xᵢ)

yₐ=mean(yᵢ)

zₐ=mean(zᵢ)

If the accelerometer is being worn on the patient's front (e.g., on the patient's chest), the remote server 102 may apply an angle transformation to the averaged accelerometer measurements. This transformation rotates the axis reference from the front location to a side location to bring the data to a common frame of reference (e.g., a common frame of reference with accelerometer data from accelerometers worn on the patient's side). To do this transformation, the remote server 102 defines rotation matrices ROT₁ and ROT₂ as follows:

$${ROT}_{1} = [ \begin{matrix}cos ( θ ) & - sin ( θ ) & 0 \\ sin ( θ ) & cos ( θ ) & 0 \\ 0 & 0 & 1\end{matrix} ] \\ {ROT}_{2} = [ \begin{matrix}1 & 0 & 0 \\ 0 & cos ( α ) & - sin ( α ) \\ 0 & sin ( α ) & cos ( α )\end{matrix} ]$$

where α=24° and θ=45°. The remote server 102 then rotates the acceleration vector as follows:

$$[ \begin{matrix}{x}_{r} \\ {y}_{r} \\ {z}_{r}\end{matrix} ] = [ \begin{matrix}{x}_{a} \\ {y}_{a} \\ {z}_{a}\end{matrix} ] * {ROT}_{1} * {ROT}_{2}$$

Other similar transformations may be applied if the accelerometer is worn elsewhere, such as on the patient's back.

For accelerometer data normalized to a common frame of reference, the remote server 102 may then calculate pitch and roll using the formulas:

$$pitch = a tan 2 ( - {y}_{r} , \sqrt{{x}_{r}^{2} + {z}_{r}^{2}} ) \\ roll = a tan 2 ( {z}_{r} , \sqrt{{y}_{r}^{2} + μ * {x}_{r}^{2}} )$$

where atan 2 is the four quadrant inverse tangent, μ=0.1, and μ*ᵣₓ² is used to remove calculation instabilities. Additionally, in the formula above, the pitch angle is the tilt angle from a vertical orientation. This angle is used to determine if the patient 104 is supine, reclined, or upright.

In some embodiments, the remote server 102 determines the posture information by classifying the patient's posture over time into various posture types. The posture types may include, for example, “supine,” “reclined,” “upright,” “standing,” “sitting,” “resting,” “lying on the right side,” “lying on the left side,” and/or the like. To illustrate, the remote server 102 may either determine or receive from the motion sensor a signal indicating the device tilt of the motion sensor in degrees over time. The remote server 102 may compare the degree of the motion sensor's tilt to various degree thresholds to classify whether a patient's posture at any given time fits under various categories, such as “supine,” “reclined,” and “upright.” For instance, the remote server 102 may determine that the patient 104 was supine where the motion sensor's tilt (e.g., the patient's pitch angle) was less than 30 degrees, determine that the patient 104 was reclined when the motion sensor's tilt was between 30 degrees and 60 degrees, and determine that the patient was upright when the motion sensor's tilt was greater than 60 degrees.

The posture for the patient 104 may also be calibrated, e.g., the first time that the patient 104 wears the wearable cardiac monitoring device 100 to determine an upright posture for the patient 104. As an example, the patient 104 may wear the wearable cardiac monitoring device 100 in a caregiver's office (e.g., an office of the clinician). The caregiver may indicate to the wearable cardiac monitoring device 100, or directly to the remote server 102 via a clinician-authorized user terminal 106, when the patient 104 is upright versus reclined. The remote server 102 may then set degree thresholds for classifying posture based on this calibration.

As another example, a motion detector on the wearable cardiac monitoring device 100 may include an accelerometer, and the motion data may capture activity information for the patient 104. In particular, activity may be measured in accelerometer counts, which are correlated with energy expenditure due to physical activity (e.g., a commonly accepted measure of activity level). Counts may be calculated by taking patient acceleration, removing the component exerted by the Earth's gravity, taking the absolute value, and integrating over time. In some implementations, determining the activity information for the patient 104 may include determining accelerometer counts for a certain amount of time. For instance, the number of accelerometer counts per second may be determined. As another example, the number of accelerometer counts per second may be determined and multiplied by 60 to find the number of accelerometer counts per minute at any given time for the patient 104. As another example, these number of accelerometer counts per minute may be averaged over the course of a certain period of time, such as five minutes, to determine an average number of accelerometer counts over the period of time. In some cases, the count determination may be made at the remote server 102, which may receive raw or filtered accelerometer signals from the motion sensor. In other cases, this determination may be made by the motion sensor of the cardiac monitoring device 100, which transmits motion data that include accelerometer counts corresponding to activity information for the patient 104 over time to the remote server 102.

In implementations, the remote server 102 may apply step count estimation using the following criteria to identify step candidates: 1) an acceleration value greater than a threshold, 2) a gap between steps with acceleration lower than a threshold, 3) a minimal time gap between steps, and 4) the peak acceleration being similar for raw data peaks and lowpass filtered peaks. To detect missed steps, the remote server 102 may calculate the time gaps between step candidates. In case of a large gap, relative to the neighboring steps, the remote server 102 may look for a peak that might have been missed between the two respective steps. To avoid double counting steps that may have a multi-peak pattern, the remote server 102 may distribute the time gaps between the steps.

In some embodiments, the remote server 102 determines the activity information by classifying the patient's activity level over time into various activity level types. The activity level types may include, for example, “active,” “non-active,” “walking,” “low activity,” “intermediate activity,” “high activity,” “patient fall,” and/or the like. As an illustration, the remote server 102 may either determine or receive, from the motion sensor, signals indicating the accelerometer counts of the motion sensor over time. The remote server 102 may compare the accelerometer counts to various count thresholds to classify whether a patient's activity level at any given time fits under various categories, such as “active” and “non-active.” For instance, the remote server 102 may determine that the patient 104 was active when the accelerometer counts per minute were 1000 or more and that the patient 104 was non-active when the accelerometer counts per minute were less than 1000. To illustrate another example, the remote server 102 may determine that the patient 104 was active when the accelerometer counts per second were greater than 15 for at least 30 seconds of a minute time interval and that the patient 104 was non-active the rest of the time.

As another illustration, the remote server 102 may determine that the patient 104 was in an “active” state for a given minute when the patient 104 showed activity (e.g., accelerometer counts above a predetermined threshold) for at least 30 seconds and had an average posture greater than 35 degrees. The remote server 102 may determine that the patient 104 was in an “asleep” state for a given minute when the patient 104 showed activity for less than 12 seconds and had an average posture less than or equal to 35 degrees for that minute. The remote server 102 may finally determine that the patient 104 was in an “inactive” state if the patient 104 had a combination of activity and posture values for a given minute that did not satisfy the active or sleep state thresholds.

As another example, a motion detector on the wearable cardiac monitoring device 100 may include an accelerometer, and the motion data may capture respiration information for the patient 104. More specifically, after signal filtering (e.g., filtering the motion signals from the accelerometer to isolate low-frequency portions of the signals), the filtered motion signals may be divided into recording intervals. Each recording interval may be checked for regularity characteristic of breathing cycles. This regularity may be marked, for instance, by a combination of requirements regarding signal peak and dip levels and distribution. For example, each recording interval may be compared to one or more breathing cycle templates to identify breathing cycles, and a certain number of breathing cycles may be compared to one another to determine if there is regularity in their signal peaks and dip levels. Once a “regular” stretch is confirmed, inhalation peaks are detected and counted. Respiration rate is then calculated as the average time interval between consecutive peaks in the regular stretch. In some cases, this respiration rate determination may be made at the remote server 102, which may receive raw or filtered accelerometer signals from the motion sensor. In other cases, this determination may be made by the motion sensor of the wearable cardiac monitoring device 100, which transmits motion data to the remote server 102 that include respiration rates for the patient 104 over time.

Systems and techniques for determining respiration from triaxial accelerometer data are described below, based on “Respiratory rate and flow waveform estimation from tri-axial accelerometer data,” A. Bates, M. J. Ling, J. Mann, and D. K. Arvind, Sensors, 2016, 16, 750-756. The accelerometer is assumed to be worn on the chest of the patient 104. If the accelerometer is worn elsewhere on the patient 104 (e.g., worn on the back or the side of the patient 104), the accelerometer data may be transformed to normalize the data according to the position of the accelerometer, or the formulas below may be modified to account for the position of the accelerometer. In this case, if the patient 104 is still, then the measured and normalized acceleration vector, a, will be close to the acceleration due to gravity, g, because the linear accelerations due to breathing would be relatively small. As noted in Bates et al., “as the accelerometer rotates, the gravity vector will rotate in the co-ordinate frame of the device. The axis of this rotation may be arbitrarily oriented in the device frame, and may change due to differences in the way the patient breathes at different times. It can also change with the orientation of the patient.” More specifically, the angle θₜ and the axis of rotation rₜ between consecutive measurements of the acceleration vector at times t−1 and t were determined by dot and cross products as follows:

$${θ}_{t} = {cos}^{- 1} ( {a}_{t} \cdot {a}_{t - 1} ) \\ {r}_{t} = {a}_{t} \times {a}_{t - 1}$$

Because oscillatory rotation rₜ inverts when the direction of rotation reverses, Bates et al. normalized the axis direction by comparison to a reference axis r_{ref} as follows:

$${r}_{t}^{′} \{ \begin{matrix}{r}_{t} , & {r}_{t} \cdot {r}_{ref} \geq 0 \\ - {r}_{t ,} & {r}_{t} \cdot {r}_{ref} < 0\end{matrix}$$

The predominant axis of rotation was then identified from measurements over a window of length W. Additionally, to reduce the noise, the estimate was weighted by the angle θₜ associated with each measurement, and to weight measurements closer in time to t, a Hamming window function H(n) was used. This process may be represented as follows:

$${\overline{r}}_{t} = normalize ( {\sum }_{i = - W / 2}^{W / 2} H ( i ) {θ}_{t + i} {r}_{t + i}^{′} )$$

The central point of the present rotations was estimated similarly:

$${\overline{a}}_{t} = normalize ( {\sum }_{i = - W / 2}^{W / 2} {a}_{t + i} )$$

Using these values, the current rotation angle ϕₜ, measured from the mean direction of gravity, was determined as follows:

$${ϕ}_{t} = {sin}^{- 1} ( ( {\overline{a}}_{t} \times {\overline{r}}_{t} ) \cdot {a}_{t} )$$

The angular velocity on the axis ωₜ was then determined as the derivative of ϕₜ:

$${ω}_{t} \frac{d ϕ}{dt}$$

The angular rate in radians per second is used to determine the respiration rate. For example, thresholds in the angular rate signals, scaled based on the RMS level of the signal, and dividing the amplitude range of the signal into low, middle and high bands can be determined. Accordingly, identification of a complete breath can be established using a state machine, where the signal transitions through a middle, a high, the middle, a low, and then middle bands, in order to register a breath. The instantaneous respiratory rate is then given by the number of such qualifying breaths within a predetermined period of time.

In some embodiments, the remote server 102 determines the respiration information by classifying the patient's respiration over time into various respiration types. The respiration types may include, for example, “regular breathing,” “rapid breathing,” “shallow breathing,” “rapid shallow breathing,” “deep breathing,” “wheezing,” “sleep disordered breathing,” “Cheyne-Stokes respiration,” and/or the like. As an illustration, the remote server 102 may either determine or receive from the motion sensor a signal indicating the respiration rate of the patient 104 over time. The remote server 102 may compare the respiration rates to various respiration thresholds to classify whether a patient's respiration rate at any time fits under various categories, such as “regular breathing,” “shallow breathing,” and “deep breathing.” For instance, the remote server 102 may determine that the patient 104 was experiencing regular breathing when the patient 104 was breathing between 12 and 20 breaths per minute, that the patient 104 was experiencing shallow breathing when the patient 104 when the patient 104 was breathing over 20 breaths per minute, and that the patient 104 was experiencing deep breathing when the patient 104 was breathing less than 12 breaths per minute. In some cases, the respiration thresholds used to determine which category the patient's respiration rate fits into are determined on a patient-by-patient basis, such as based on a respiration rate baseline for the patient 104 taken by a clinician.

In implementations, with respect to the wear state data, the remote server 102 may process the wear state data to determine the wear state of the wearable cardiac monitoring device 100 over time. FIG. 2C illustrates a sample process flow for monitoring for and outputting patient wear compliance information, including the wear state of the wearable cardiac monitoring device 100. For example, the sample process 250 can be implemented by the remote server 102, as described below. Alternatively, in some embodiments, the sample process 250 can be at least partially implemented by the wearable cardiac monitoring device 100, and the wearable cardiac monitoring device 100 may transmit an output of the sample process 250 to the remote server 102 or an output of part of the sample process 250 to the remote server 102 for further processing.

As shown in FIG. 2C, the remote server 102 can receive one or more sensor signals from the wearable cardiac monitoring device 100 at step 252. For example, the sensor signals can include signals from one or more physiological sensors such as an ECG sensor, an RF-based physiological sensor, a bio-acoustic sensor, a biovibrational sensor, and other similar sensors. The sensor signals can also include signals from one or more motion sensors such as accelerometers. In some examples, the signals can also include electrical signals from one or more sensors from which various electrical parameters such as impedance measurements can be measured.

A processor of the remote server 102 can monitor the received signals for an indication of a wear compliance onset event at step 254. As described herein, a wear compliance onset event, or simply an onset event, is a change in one or more monitored signals that indicates that the patient 104 has transitioned from not wearing the wearable cardiac monitoring device 100 to wearing the wearable cardiac monitoring device 100. For example, as noted herein, when a predetermined threshold based on ECG signal information is met, the patient 104 is deemed to be wearing the wearable cardiac monitoring device 100. As an illustration, the below is an example implementation of such a feature, reproduced as a sample functional specification listing various functions and/or requirements for implementation by, for example, a processor of the remote server 102:
Find the QRS complex based on the dual criteria of the amplitude and duration of the QRS complex. In an example, use the Pan Tompkins algorithm to detect QRS. After a predetermined duration of time, called, for example, “WearTimePreOnPeriod” (e.g., 5 seconds of QRS signals or other pre-configured value, or dynamically changing value), initiate wear time (e.g., “wearTimeStart”).
Dynamic changes to Wear TimePreOnPeriod duration: If the signal is noisy (as indicated by, for example, variable ECGnoiseFlagMask), then the predetermined duration of time can be extended. For example, the predetermined duration of time can be extended to around 10 seconds to allow for more ECG samples to be collected.
Dynamic changes to predetermined duration WearTimePreOnPeriod: If QRS samples are detected for a preset portion of the WearTimePreOnPeriod duration. For example, the preset portion may be set to 80%. This means that if during 80% of the Wear TimePreOnPeriod the dual criteria is met, then compliance tracking is initiated (WearTimeStart). Otherwise, the WearTimePreOnPeriod duration is extended by an additional period, for example, 3 seconds. The above dynamic check is then repeated for the extended Wear TimePreOnPeriod duration. The WearTimePreOnPeriod resets when the total duration reaches a predetermined maximum (e.g., 15 seconds).

In certain implementations, the remote server 102 can be configured to receive a user input indicating that the patient 104 has put on the wearable cardiac monitoring device 100. Depending upon the implementation, the remote server 102 can monitor one or more additional signals to confirm that the patient 104 has put on the wearable cardiac monitoring device 100 as described herein.

During monitoring 254, the remote server 102 can determine whether an onset event has occurred at step 256. If the remote server 102 determines that an onset event has not occurred, the remote server 102 can continue to monitor 254 the electrical signals for an onset event. Conversely, if the remote server 102 does determine that an onset event has occurred, the remote server 102 can record the onset event and monitor the electrical signals for a wear compliance offset event at step 258. For example, as described herein, a wear compliance offset event, or simply offset event, may be a change in one or more monitored signals that indicates that the patient 104 has transitioned from wearing the wearable cardiac monitoring device 100 to not wearing the wearable cardiac monitoring device 100.

As further shown in FIG. 2C, during monitoring 258, the remote server 102 can determine whether an offset event has occurred at step 260. If the remote server 102 determines that an offset event has not occurred, the remote server 102 can continue to monitor the electrical signals for an offset event. Conversely, if the remote server 102 does determine that an offset event has occurred, the remote server 102 can determine wear compliance information for the period of time between the onset event and the offset event at step 262. The remote server 102 can then output the wear compliance information to, for example, a wear compliance data structure for the patient 104 at step 264. Additionally, the remote server 102 can be configured to output at least a portion of the wear compliance information in a notification to the patient 104, a caregiver of the patient 104, and/or the prescribing physician (e.g., via the patient user device 108 and/or a clinician-authorized user terminal 106). In such an example, the output notification can include any recorded changes in the patient's wear compliance that exceeds, for example, a compliance change threshold. For example, if a patient's wear compliance percentage changes by more than a particular amount in a day (e.g., between 5% and 15% between consecutive days), the notification can include information about the change in wear compliance. Monitoring wear compliance for a patient wearing a medical device may be found in U.S. patent application Ser. No. 16/951,246, entitled “Systems and Methods of Monitoring Wear Compliance of a Patient Wearing an Ambulatory Medical Device,” filed on Nov. 18, 2020, is hereby incorporated by reference in its entirety and submitted herewith as Appendix A.

Using the sample process 250, the remote server 102 may determine the wear state for the wearable cardiac monitoring device 100 over time. In implementations, the wear state may include an indication of whether the patient 104 is wearing/was wearing the wearable cardiac monitoring device 100. In implementations, the wear state may include how tightly the patient 104 wore the wearable cardiac monitoring device 100 (e.g., “wear comfortable”, “wear loose”, “wear tight”, etc.), the conditions under which the patient 104 is wearing the wearable cardiac monitoring device 100 (e.g., “wear sleep”, “wear active”, “wear shower”, “wear exercise”, “wear bathe”, etc.), the general duration of how long the patient 104 wore the wearable cardiac monitoring device 100 (e.g., “wear long duration”, “wear short duration”, etc.), and/or the like.

In implementations, the remote server 102 may process additional signals and data, such as the at least one ECG signal. For example, the remote server 102 may identify R waves in the ECG signal, and use the R waves to determine the heart rate of the patient 104 over time, the R-R intervals of the patient 104 over time, the heart rate variability of the patient 104 over time, and so on. The remote server 102 may then determine, for instance, using the processed ECG signals and the processed motion detector data the resting heart rate of the patient 104, the average R-R interval of the patient 104 (e.g., over a period of time, such as over a period of hours or days, while the patient 104 was inactive), the average heart rate variability of the patient 104 (e.g., over a period of time, such as over a period of hours or days, while the patient 104 was inactive), and so on.

As another example, the remote server 102 may process signals and data indicating whether the patient 104 performed a certain action, such as acknowledging a message or attending an appointment. For instance, the remote server 102 may receive appointment data indicating that the patient 104 had an appointment with a clinician during a particular time period (e.g., from the clinician's appointment system, from a calendar entry on the patient user device 108). The remote server 102 may then receive additional appointment data (e.g., from the clinician's health records for the patient 104, from GPS information from the patient user device 108) related to whether the patient 104 attended the appointment. The remote server 102 may process the appointment data to identify the appointment and determine whether the patient 104 attended the appointment.

As another example, the remote server 102 may process a combination of signals to determine a status or condition of the patient 104, such as processing a combination of signals to determine the sleep status of the patient 104. Sleep status for the patient 104 may be determined from, for example, a combination of posture, activity level, and/or respiration of the patient 104. For instance, the patient 104 may be determined to be asleep when the patient 104 is identified as supine (e.g., at a tilt of less than 35 degrees) and non-active (e.g., showed a non-active accelerometer count for at least 12 seconds of a minute time interval). To illustrate another example, the patient 104 may be determined to be asleep when the patient 104 is identified as supine, non-active, and experiencing deep breathing (e.g., less than 12 breaths per minute) for at least five consecutive minutes.

As another illustration, the remote server 102 may determine heart rate recovery information for the patient 104 by identifying a period of activity (e.g., a certain amount of time, such as five or ten minutes, during which the patient's accelerometer counts showed that the patient was active) and determining the patient's heart rate (e.g., determined from the ECG signals at step 232 of FIG. 2B) at one or more interval following the end of the period of activity. For example, the remote server may determine the patient's heart rate at the cessation of activity and one minute after the cessation of activity, and further determine the difference between the heart rates as the heart rate recovery. As another example, the remote server 102 may determine the patient's heart rate at the cessation of activity and 10, 20, 30, 40, 50, and 60 seconds after the cessation of activity. The remote server 102 may further determine the difference between the heart rates at cessation and each of the 10-second intervals after the cessation of activity as the patient's heart rate recovery.

Processing the motion data, wear state data, and/or other patient data 222 may include storing the processed data in a data structure, such as the patient incentive data structure or another data structure for the patient 104. To illustrate, the remote server 102 may generate a data structure that includes wear state data for the patient 104 over time, such as how much time the patient 104 wore the wearable cardiac monitoring device 100 for each day of a predetermined time period (e.g., 10 days, 20 days, 30 days, 45 days, 60 days, 90 days, 180 days, etc.).

The remote server 102 identifies the first incentive criterion from the stored patient incentive data structure at step 224. For example, the remote server 102 retrieves the first incentive criterion entry in the patient incentive data structure, where the entry defines the first incentive criterion (e.g., though a call function formatted to output whether the patient 104 has met the incentive criterion as submitted by the patient's clinician) or points to the first incentive criterion stored in another location (e.g., through a pointer indicating a call function to be used to determine whether the patient 104 has met the incentive criterion as submitted by the patient's clinician). As another example, the remote server 102 may identify whether a data structure for a particular incentive criterion or type of incentive criteria, where the data structure includes entries for a number of patients related to the particular incentive criterion or type of incentive criteria, includes an entry for the patient 104.

The remote server 102 determines, based on the processed data from step 222, whether the retrieved incentive criterion is satisfied at step 226. In implementations, the remote server 102 may execute a call function that outputs whether the patient 104 has satisfied the incentive criterion and/or outputs the amount of reward related to the incentive criterion that the patient 104 receives.

For example, the remote server 102 may determine whether the patient 104 has met a physical activity goal (e.g., a number of steps over a predetermined time period and/or a number of minutes of physical activity over a predetermined time period) based on the processed motion detector data and/or processed ECG signal. As another example, the remote server 102 may determine whether the patient 104 has met a health goal (e.g., a resting heart rate goal, an R-R interval goal, a heart rate variability goal, etc.) based on the processed motion detector data and/or processed ECG signal. As another example, the remote server 102 may determine whether the patient 104 has met a health improvement goal based on the processed motion detector data and/or processed ECG signal and further based on a starting health value (e.g., starting resting heart rate, starting R-R interval, starting heart rate variability, starting exercise level, etc.) measured or otherwise taken from the patient 104 at the beginning of the period the patient 104 started wearing the wearable cardiac monitoring device 100. As another example, the remote server 102 may determine whether the patient 104 has met a wear compliance goal (e.g., a goal to wear the wearable cardiac monitoring device 100 for at least a certain number of hours per day and/or per week) based on the processed wear state data. As another example, the remote server 102 may determine whether the patient 104 has met a sleep goal based on the processed motion detector data and/or processed ECG data (e.g., processed to determine when the patient 104 was awake versus sleeping, as described above).

As another example, the remote server 102 may determine whether the patient 104 has acknowledged a message sent to the patient (e.g., a text message, a content chapter the patient 104 was requested to read, a landing page the patient 104 was requested to visit, an educational video the patient 104 was requested to watch, a questionnaire the patient 104 was requested to fill out, etc.) based on processed message acknowledgement data for the patient 104. As another example, the remote server 102 may determine whether the patient 104 attended an appointment based on processed appointment data for the patient 104. As another example, the remote server 102 may determine whether the patient shared information related to a health status of the patient 104 and/or a status of the wearable cardiac monitoring device 100 with a caregiver based on processed information sharing data for the patient 104.

If the remote server 102 determines that the patient 104 has satisfied the incentive criterion, the remote server 102 updates the patient incentive data structure to indicate that the patient 104 has earned a complete incentive reward at step 228. For example, the remote server 102 may determine the type and/or amount of reward that the patient 104 has earned by completing the incentive criterion (e.g., based on information for the reward stored in the patient incentive data structure, based on standardized or default reward information for the satisfied incentive criterion stored elsewhere, etc.). The remote server 102 may then create, fill out, or update an entry in the patient incentive data structure to indicate the reward that the patient 104 has earned. As another example, the information for the reward that the patient 104 can earn by completing the incentive criterion may be already stored in the patient incentive data structure, and the remote server 102 may update the patient incentive data structure to indicate that the patient 104 has earned the reward.

In implementations, updating the patient incentive data structure may include determining whether the patient has reached a rewards limit. As an illustration, the default reward for a patient clicking on a text or SMS message may be one point, but the incentive criteria may include a limit (e.g., a default limit or a limit imposed by the patient's clinician) on the number of points the patient 104 can receive for clicking on a text message over a predetermined time period, such as a day, week, or month. For instance, the patient 104 may be limited to eight points from clicking on text messages per week. Accordingly, if the remote server 102 determines that the patient has clicked on a text message (e.g., based on message acknowledgement data), the remote server 102 may determine if the patient has already received eight points for clicking on text messages within the calendar week. If the patient 104 has not reached the eight-point limit for the calendar week, the remote server 102 updates the patient incentive data structure to indicate that the patient 104 has earned a point for clicking on the text message. However, if the patient has already reached the eight-point limit for the calendar week, the remote server 102 does not update the patient incentive data structure. In some examples, if the remote server 102 determines that the patient 104 has reached the eight-point limit, the remote server 102 updates the patient incentive data structure to indicate that the patient has received eight points from clicking on text messages but that the patient 104 cannot receive further points for clicking on text messages until the next calendar week.

After updating the patient incentive data structure 228, at step 234, the remote server 102 moves on to determine if there is an additional incentive criterion for the patient 104 that the remote server 102 has not yet determined whether the patient 104. Otherwise, if the patient 104 has not satisfied the incentive criterion at step 226, the remote server 102 determines whether the patient 104 has partially satisfied the incentive criterion at step 230. In implementations, determining whether the patient 104 has partially satisfied the incentive criterion may be a separate step from determining whether the patient has completely satisfied the incentive criterion 226 (e.g., based on the remote server 102 executing a separate function). In implementations, determining whether the patient 104 has partially satisfied the incentive criterion may be part of determining whether the patient has completely satisfied the incentive criterion 226 (e.g., part of executing the function to determine whether the patient 104 has completely satisfied the incentive criterion 226).

In implementations, as discussed above, the patient incentive data structure may include information on partial rewards that the patient 104 may earn by partially satisfying a given incentive criterion. As an illustration, the patient incentive data structure may store an indication that the patient 104 receives two ribbons for completing 30 minutes of physical activity in a day and that the patient 104 receives one ribbon for completing 20 minutes of physical activity in a day. Accordingly, if the remote server 102 determines that the patient has not completed 30 minutes of physical activity in a day, the remote server 102 may move on to determining whether the patient 104 has completed 20 minutes of exercise in the day and has thus earned a partial reward.

In implementations, the remote server 102 determining whether the patient 104 has partially satisfied the incentive criterion 230 may include determining whether the patient has satisfied a predetermined proportion of the incentive criterion. For instance, if the incentive criterion is to wear the wearable cardiac monitoring device 100 for a predetermined number of hours per day, the remote server 102 may determine the proportion of the predetermined number of hours that the patient 104 wore the wearable cardiac monitoring device 100 for a given day. The partial reward may be the same as the proportion of the incentive criterion that the patient 104 satisfied. To illustrate, if the patient 104 is prescribed to wear the wearable cardiac monitoring device 100 for 20 hours per day, the patient 104 may receive one point for every four hours that the patient 104 wore the wearable cardiac monitoring device 100 in the day, for a maximum of five points. In some examples, the patient 104 may only receive a reward if the patient has completed at least a predetermined proportion of the incentive criterion. Referring to the previous illustration, the patient 104 may receive one point for every four hours that the patient 104 wore the wearable cardiac monitoring device 100 in a day, but the accrued points may only be rewarded to the patient if the patient wore the wearable cardiac monitoring device 100 for at least 10 hours in the day. Alternatively, in some embodiments, the partial reward may be different, such as less than, the proportion of the incentive criterion that the patient 104 has satisfied. Again referring to the previous illustration, the patient 104 may receive five points for wearing the wearable cardiac monitoring device 100 for at least 20 hours in a day and receive two points for wearing the wearable cardiac monitoring device 100 for at least 16 hours in the day.

In implementations, the remote server 102 may award partial rewards to the patient 104 based on how many consecutive days, weeks, etc. the patient 104 has satisfied the incentive criterion 230. Alternatively, the remote server 102 may award partial rewards to the patient 104 based on a proportion of days in a predetermined time span, such as a week, ten days, two weeks, etc., that the patient 104 satisfied the incentive criterion 230. The partial rewards may scale non-linearly with the number of days the patient 104 fulfilled the incentive criterion 230. For instance, if the patient 104 wore the wearable cardiac monitoring device 100 for at least 20 hours in a day for seven consecutive days in the previous week, the remote server 102 may award the patient 104 with seven points. If the patient 104 wore the wearable cardiac monitoring device 100 for at least 20 hours in a day for five to six consecutive days in the previous week, the remote server 102 may award the patient 104 with three points. If the patient 104 wore the wearable cardiac monitoring device 100 for at least 20 hours in a day for three to four consecutive days in the previous week, the remote server 102 may award the patient 104 with one point. If the patient 104 wore the wearable cardiac monitoring device 100 for at least 20 hours in a day for two or less consecutive days in the previous week, the patient 104 may receive no points.

If the remote server 102 determines that the patient 104 has partially satisfied the incentive criterion, the remote server 102 updates the patient incentive data structure to indicate that the patient has earned a partial incentive reward at step 232. Step 232 may be performed similarly to step 228, as discussed above. After updating the patient incentive data structure, at step 234, the remote server 102 then identifies if there is an additional incentive criterion that the remote server 102 has not yet determined whether the patient 104 has satisfied. If there is an additional incentive criterion, the remote server 102 identifies the next incentive criterion from the stored patient incentive data structure at step 236 and repeats step 226 for the next incentive criterion. Otherwise, if the remote server 102 identifies that the remote server 102 has determined whether or not the patient satisfies all the incentive criteria stored for the patient 104, the remote server 102 returns to monitoring for signals and data for the patient 104 at step 206 of FIG. 2A. If instead the remote server 102 determines that the patient 104 has not partially satisfied the incentive criterion at step 230, the remote server 102 identifies if there is an additional incentive criterion that the remote server 102 has not yet determined whether the patient 104 has satisfied at step 234, as discussed above.

In some implementations, the remote server 102 may not provide the patient 104 with partial rewards. As such, if the patient 104 has not satisfied the incentive criterion, the remote server may not perform steps 230 and 232 and skip directly to step 234 after updating the patient incentive data structure to indicate that the patient has earned a complete incentive reward at step 228 or after determining that the patient 104 has not satisfied the incentive criterion at step 226.

In some implementations, the remote server 102 may execute the sample process 220 periodically, such as daily at a predetermined time of day. In some implementations, the remote server 102 may execute the sample process 220 continuously. In some implementations, the remote server 102 may execute the sample process 220 upon a trigger, such as upon receiving signals and/or data from the wearable cardiac monitoring device 100.

In some implementations, the remote server 102 may be configured to receive more than one set of incentive criteria for the patient 104. For example, the remote server 102 may receive a first set of incentive criteria for the patient 104 at a first time and receive a second set of incentive criteria for the patient at a second time. The remote server 102 may then update the patient incentive data structure for the ambulatory cardiac patient 104 based on the second set of incentive criteria (e.g., similar to the process described above with respect to step 204 of FIG. 2A). As another example, the remote server 102 may receive a first set of incentive criteria for the patient 104 defining a first goal or set of goals and, at the same time, a second set of incentive criteria for the patent 104 defining a second goal or set of goals. As an illustration, a first set of goals may be related to the physical health of the patient 104, and the second set of goals may be related to actions the patient 104 is requested to perform. The remote server 102 may update the patient incentive data structure for the ambulatory cardiac patient 104 based on the second set of goals, as described above. After the patient incentive data structure is updated, the remote server 102 may execute steps 206-214 of FIG. 2A and sample process 220 of FIG. 2B for all of the incentive criteria stored in the patient incentive data structure, including both sets of incentive criteria.

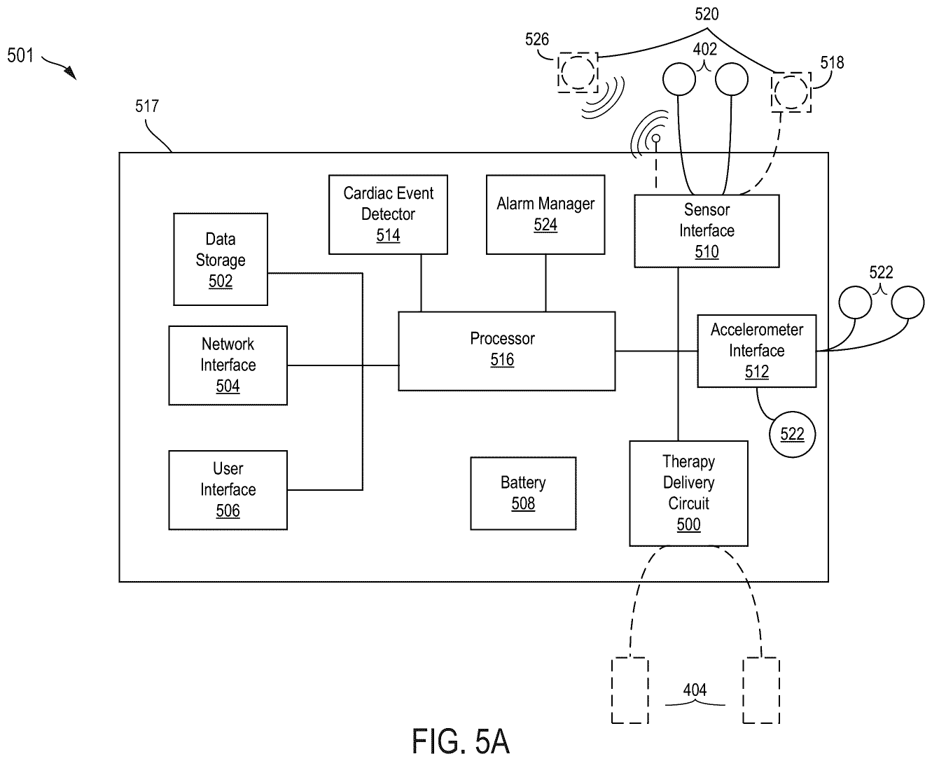

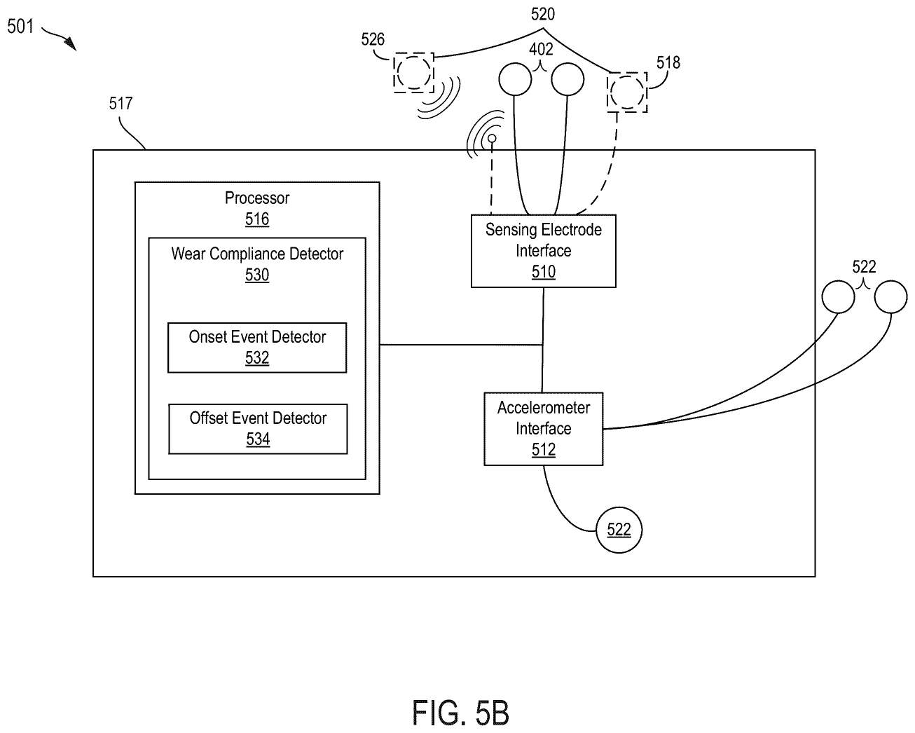

An example of a patient incentive data structure is shown below as Table 1. In this example data structure, a first column includes entries of patient ID numbers corresponding to patients prescribed to wear wearable cardiac monitoring devices 100. A second column includes entries of the prescription period for each patient. A third column includes entries of the number of days each patient in the data structure has met a daily goal (e.g., defined by the incentive criteria for each patient) for wearing the wearable cardiac monitoring device 100 for a predetermined amount of time. Similarly, a fourth column includes entries of the number of days each patient in the data structure has partially met the daily goal for wearing the wearable cardiac monitoring and treatment device 100 for a predetermined amount of time. The remote server 102 may generate the entries for the third and fourth columns by, e.g., using impedance data from an impedance circuit of each wearable cardiac monitoring device 100 to determine when each patient was wearing the device 100. The remote server 102 determining the number of days a patient has met a goal to wear the device 100 for a predetermined amount of time is described in further detail above with respect to FIGS. 2B and 2C and below with respect to FIG. 5B.

TABLE 1

| | | | Number of days |
|---|---|---|---|
| | Total device | Number of days | patient has |
| | prescribed | patient has met | partially met |
| Patient ID | period (days) | wear goal (days) | wear goal (days) |
| P00233545 | 90 | 23 | 30 |
| P00233546 | 90 | 36 | 44 |
| P00233547 | 90 | 34 | 34 |
| P00233548 | 45 | 39 | 39 |
| P00233549 | 60 | 57 | 58 |
| P00233550 | 60 | 54 | 54 |
| P00233551 | 45 | 39 | 39 |
| P00233552 | 45 | 40 | 40 |
| P00233553 | 90 | 78 | 81 |
| P00233554 | 180 | 150 | 161 |
| P00233555 | 180 | 145 | 145 |
| P00233556 | 180 | 23 | 40 |
| P00233557 | 90 | 23 | 23 |
| P00233558 | 90 | 34 | 34 |
| P00233559 | 180 | 39 | 72 |
| P00233560 | 45 | 32 | 32 |
| P00233561 | 60 | 54 | 54 |
| P00233562 | 60 | 39 | 43 |
| P00233563 | 45 | 44 | 44 |
| P00233564 | 90 | 23 | 32 |
| P00233565 | 90 | 23 | 23 |
| P00233566 | 90 | 34 | 44 |
| P00233567 | 45 | 39 | 39 |
| P00233568 | 60 | 57 | 58 |
| P00233569 | 60 | 12 | 19 |
| P00233570 | 45 | 39 | 39 |

Table 2 below illustrates a continuation of the data structure of Table 1. As shown in Table 2, the example data structure includes a fifth column of the percentage of days each patient has met the wear goal compared to the total number of days in the prescribed period of time (e.g., the percentage of column 3 to column 2 of Table 1 above). Similarly, a sixth column includes the percentage of days each patient has partially met the wear goal compared to the total number of days in the prescribed period of time (e.g., the percentage of column 4 to column 2 of Table 1 above).

TABLE 2

| Percentage of days | Percentage of days wear | Calculated |
|---|---|---|
| wear goal met to | goal partially met to | incentives |
| prescribed period | prescribed period | (ribbons) |
| 25.5556 | 33.3333 | 60 |
| 40.0000 | 48.8889 | 89 |
| 37.7778 | 37.7778 | 76 |
| 86.6667 | 86.6667 | 174 |
| 95.0000 | 96.6667 | 192 |
| 90.0000 | 90.0000 | 180 |
| 86.6667 | 86.6667 | 174 |
| 88.8889 | 88.8889 | 178 |
| 86.6667 | 90.0000 | 178 |
| 83.3333 | 89.4444 | 175 |
| 80.5556 | 80.5556 | 162 |
| 12.7778 | 22.2222 | 36 |
| 25.5556 | 25.5556 | 525 |
| 37.7778 | 37.7778 | 762 |
| 21.6667 | 40.0000 | 63 |
| 71.1111 | 71.1111 | 144 |
| 90.0000 | 90.0000 | 180 |
| 65.0000 | 71.6667 | 137 |
| 97.7778 | 97.7778 | 196 |
| 25.5556 | 35.5556 | 62 |
| 25.5556 | 25.5556 | 52 |
| 37.7778 | 48.8889 | 88 |
| 86.6667 | 86.6667 | 174 |
| 95.0000 | 96.6667 | 192 |
| 20.0000 | 31.6667 | 52 |
| 86.6667 | 86.6667 | 174 |

A seventh column includes the calculated number of incentives, such as ribbons, for each patient listed in the data structure. The calculated number of incentives for each patient is based on the percentage of days each patient has completely or partially met the daily goal of wearing the wearable cardiac monitoring device 100 for the predetermined amount of time (e.g., according to each patient's incentive criteria). For example, in Table 2, the calculated incentives for each patient was determined by rounding up the percentage of days the wear goal was completely met in the fifth column and multiplying this number by two. Then, the difference between the percentage of days the wear goal was completely met in the fifth column was subtracted from the percentage of days the wear goal was partially met in the sixth column. This difference was rounded up and added to the rounded up percentage of days the wear goal was completely met. In other words, the remote server 102 awarded each patient of the data structure two ribbons for the percentage of days the patient has completely met the wear goal and one ribbon for the additional percentage of days the patient has partially met the wear goal. As such, patients are rewarded more for completely meeting the wear goal than for only partially meeting the wear goal. Additionally, because the calculated incentives are based on the percentage of days the wear goal has been completely or partially met, the calculated incentives are standardized across the patients such that, for example, a patient with higher and lower prescription periods have the same capacity to earn ribbons. The formula used to determine the entries for the seventh column is also provided below:

$$Calculated incentive = ROUNDUP ( \frac{Days goal met}{Days of prescription} ) * 2 + ROUNDUP ( \frac{Days goal partially met}{Days of prescription} - \frac{Days goal met}{Days of prescription} )$$

Another example of a patient incentive data structure is shown in Table 3 below. The example data structure of Table 3 includes a first column of entries of patient ID numbers corresponding to patients prescribed to wear wearable cardiac monitoring devices 100. A second column includes entries of the prescription period for each patient. A third column includes entries of which day of the prescription period each patient of the data structure is currently on. A fourth column includes entries of a cohort number for each patient of the data structure. As described in further detail below, each patient may be grouped into a predetermined cohort of patients prescribed to wear the wearable cardiac monitoring device 100. Each patient may be shown how their progress towards completing their incentive criteria and/or earning incentive rewards compares to the other members of their cohort. For example, as shown in Table 3, patients may be grouped into a cohort depending on when they started their prescription (as illustrated in the third column) and/or how many total days they've been prescribed to wear the wearable cardiac monitoring device 100 (as shown in the second column). As an illustration, patients in Table 3 who have a prescription for between 45-90 days and are on day 43-46 of their prescription are grouped in cohort number 5, patients who have a prescription for 180 days and are on day 42-45 of their prescription are grouped in cohort number 6, and patients who have a prescription for 45-90 days and are on day 40-41 of their prescription are grouped in cohort number 7.

TABLE 3

| | Total device | Day of prescribed | Cohort |
|---|---|---|---|
| Patient ID | prescribed period | period | number |
| P00445890 | 90 | 45 | 5 |
| P00445891 | 90 | 45 | 5 |
| P00445892 | 90 | 45 | 5 |
| P00445893 | 45 | 44 | 5 |
| P00445894 | 60 | 44 | 5 |
| P00445895 | 60 | 44 | 5 |
| P00445896 | 45 | 44 | 5 |
| P00445897 | 45 | 43 | 5 |
| P00445898 | 90 | 43 | 5 |
| P00445899 | 180 | 43 | 6 |
| P00445900 | 180 | 43 | 6 |
| P00445901 | 180 | 43 | 6 |
| P00445902 | 90 | 43 | 5 |
| P00445903 | 90 | 42 | 7 |
| P00445904 | 180 | 42 | 6 |
| P00445905 | 45 | 42 | 7 |
| P00445906 | 60 | 42 | 7 |
| P00445907 | 60 | 42 | 7 |
| P00445908 | 45 | 42 | 7 |
| P00445909 | 90 | 41 | 7 |
| P00445910 | 90 | 41 | 7 |
| P00445911 | 90 | 41 | 7 |
| P00445912 | 45 | 41 | 7 |
| P00445913 | 60 | 41 | 7 |
| P00445914 | 60 | 41 | 7 |
| P00445915 | 45 | 40 | 7 |

Table 4 below illustrates a continuation of the data structure of Table 3. A fifth column, as shown in Table 4, includes entries of a daily step goal for each patient. The daily step goal may be set as part of the incentive criteria for each patient (e.g., as described above with reference to FIGS. 2A, 12, and 13). A sixth column includes entries of how many steps the patient walked the previous day. The remote server 102 may determine the steps the patient walked the previous day, for example, as described above with respect to FIG. 2B above. A seventh column includes entries of whether each patient met their step goal the previous day. As an illustration, the remote server 102 may determine whether it is “TRUE” or “FALSE” that the total steps the patient walked the previous day is greater than or equal to the daily step goal for the patient.

TABLE 4

| Daily step | Steps patient | Did patient |
|---|---|---|
| goal for patient | walked previous day | meet step goal? |
| 4000 | 4567 | TRUE |
| 4000 | 3456 | FALSE |
| 5000 | 6900 | TRUE |
| 7000 | 10894 | TRUE |
| 4000 | 1278 | FALSE |
| 5500 | 4578 | FALSE |
| 7000 | 7836 | TRUE |
| 8000 | 7635 | FALSE |
| 3000 | 1782 | FALSE |
| 3500 | 5632 | TRUE |
| 4000 | 4829 | TRUE |
| 6000 | 6823 | TRUE |
| 2000 | 3899 | TRUE |
| 5500 | 5672 | TRUE |
| 6000 | 5782 | FALSE |
| 7000 | 2893 | FALSE |
| 7000 | 8263 | TRUE |
| 7000 | 9832 | TRUE |
| 4000 | 3788 | FALSE |
| 4000 | 2722 | FALSE |
| 5000 | 6371 | TRUE |
| 5000 | 7220 | TRUE |
| 5000 | 3987 | FALSE |
| 8000 | 8217 | TRUE |
| 7000 | 7709 | TRUE |
| 4000 | 5710 | TRUE |

Table 5 below illustrates a further continuation of the data structure shown in Tables 3 and 4. As shown in Table 5, an eighth column includes entries of the total days each patient has met their step goal. For example, when the remote server 102 updates the data structure shown in Tables 3-5 (e.g., on a daily basis), the remote server 102 may re-determine whether each patient met their step goal the previous day, and if a given patient did meet their step goal, the remote server 102 may increase the total number of days the patient has met their step goal by one. Similarly, a ninth column includes entries of the total days each patient met their step goal over the previous week (e.g., the previous calendar week). As an illustration, the remote server 102 may filter all entries for whether the patient met their step goal (e.g., as stored in entries of the data structure of Tables 3-5 not shown here, or as stored in another data structure) to entries for the previous calendar week. The remote server 102 may determine how many “TRUE” entries the patient had for the previous week and enter that number in the corresponding entry for the patient in the ninth column.

TABLE 5

| | | Total | Weekly |
|---|---|---|---|
| Total days | Total days patient | calculated | calculated |
| patient has | met step goal | incentives | incentives |
| met step goal | previous week | (ribbons) | (ribbons) |
| 43 | 7 | 86 | 14 |
| 38 | 6 | 76 | 12 |
| 45 | 7 | 90 | 14 |
| 44 | 7 | 88 | 14 |
| 39 | 6 | 78 | 12 |
| 41 | 7 | 82 | 14 |
| 42 | 7 | 84 | 14 |
| 34 | 3 | 68 | 6 |
| 40 | 6 | 80 | 12 |
| 43 | 7 | 86 | 14 |
| 42 | 7 | 84 | 14 |
| 40 | 6 | 80 | 12 |
| 43 | 7 | 86 | 14 |
| 42 | 7 | 84 | 14 |
| 33 | 5 | 66 | 10 |
| 38 | 6 | 76 | 12 |
| 42 | 7 | 84 | 14 |
| 42 | 7 | 84 | 14 |
| 30 | 4 | 60 | 8 |
| 28 | 5 | 56 | 10 |
| 40 | 7 | 80 | 14 |
| 39 | 6 | 78 | 12 |
| 13 | 2 | 26 | 4 |
| 41 | 7 | 82 | 14 |
| 41 | 7 | 82 | 14 |
| 39 | 7 | 78 | 14 |

As shown in Table 5, a tenth column includes entries of the total calculated incentives, such as ribbons, each patient of the data structure has earned from completing their daily step count goal. In the example of Table 5, each day a patient successfully meets their daily step count goal, the patient earns two ribbons. Thus, the total calculated incentives (e.g., in ribbons) is twice the number of total days the patient has met their step goal (e.g., as shown in the eighth column). An eleventh column includes entries of the weekly calculated incentives (e.g., for the previous calendar week) that each patient has earned. For instance, the remote server 102 may populate the entries for the eleventh column by multiplying the total number of days in the previous week that the patient has met their step goal (e.g., as shown in the ninth column) by two.

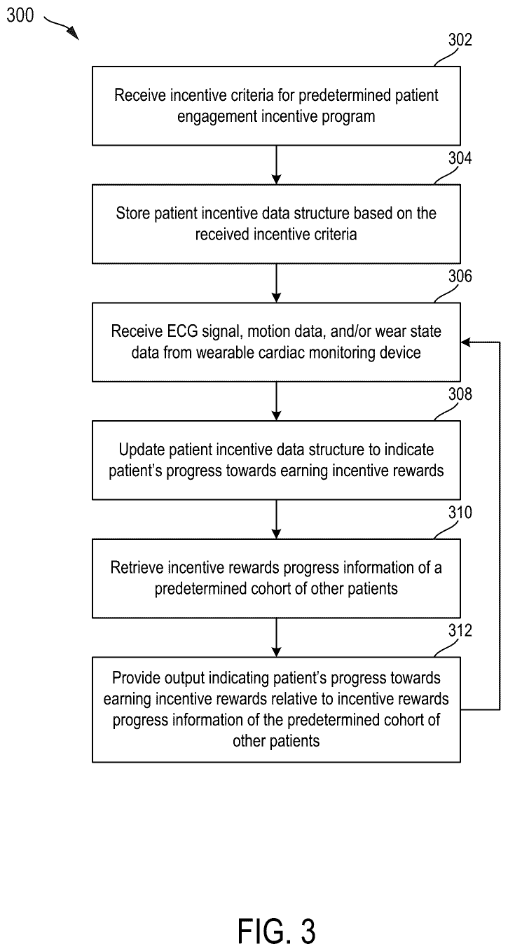

FIG. 3 illustrates a sample process flow for managing patient engagement incentives for ambulatory cardiac patients being monitored for cardiac arrhythmias. The sample process 300 shown in FIG. 3 can be implemented by a remote server 102. As shown in FIG. 3, the remote server 102 receives incentive criteria at step 302. In various embodiments, step 302 may be implemented using the sample processes described above with respect to step 202 of FIG. 2A. The remote server 102 stores a patient incentive data structure based on the received criteria at step 304. In various embodiments, step 304 may be implemented using the sample processes described above with respect to step 204. The remote server 102 receives at least one ECG signal, motion data, and/or wear state data from the wearable cardiac monitoring device 100 over a period of time at step 306. In various embodiments, step 306 may be implemented using the sample processes described above with respect to step 206 of FIG. 2A.

The remote server 102 updates the patient incentive data structure to indicate the patient's progress towards earning incentive rewards at step 308. In various embodiments, step 308 may be implemented using the sample processes described above with respect to steps 208, 210, 212, and 214 of FIG. 2A, sample process 220 of FIG. 2B, and sample process 250 of FIG. 2C. Additionally, in some implementations, the remote server 102 may update the patient incentive data structure to indicate the patient's partial progress towards earning incentive rewards using similar processes to those described above with respect to these sample processes of FIGS. 2A-2C. For example, if the patient 104 has not earned a reward yet but has made partial progress towards earning the reward, the remote server 102 may update the patient incentive data structure to indicate the patient's progress so far towards earning the reward, such as by updating the patient incentive data structure to indicate a percentage or proportion of an incentive criterion that the patient 104 has completed. As an illustration, if one of the patient's incentive criteria is lowering their resting heart rate by 5 bpm, and the patient has lowered their resting heart rate by 3 bpm, the remote server 102 may update the patient incentive data structure to indicate that the patient is 60% of the way to earning the incentive reward for the incentive criterion.

In implementations, the patient's progress towards earning incentive rewards may include determining milestones that the patient 104 may have met with earned rewards. For instance, a milestone may be a total amount of rewards that the patient 104 has earned (e.g., with milestones occurring at predetermined amounts of total ribbons earned, such as 25, 50, 75, 100, etc. total points or ribbons earned). As another example, a milestone may be a total amount of rewards that the patient 104 has earned in a particular category. To illustrate, the patient 104 may reach a milestone when the patient 104 has earned a predetermined amount of rewards for acknowledging messages, when the patient 104 has earned a predetermined amount of rewards for daily compliance with wearing the wearable cardiac monitoring device 100, when the patient 104 has earned a predetermined amount of rewards for daily exercise, etc.

The remote server 102 retrieves incentive rewards progress information of a predetermined cohort of other patients at step 310. For example, the predetermined cohort of other patients may be, may include, or may be a subset of the other patients 110 wearing cardiac monitoring devices 100 shown in FIG. 1. Each of the other patients 110 may also be associated with a patient incentive data structure updated with the respective patient's progress towards earning rewards and/or partial rewards, as described above.

In implementations, retrieving the incentive rewards progress information for the predetermined cohort of other patients may include identifying the predetermined cohort of other patients. For example, the predetermined cohort of other patients may be patients seeing the same clinician as the patient 104 and also prescribed to wear a wearable cardiac monitoring device 100. As another example, the predetermined cohort of other patients may be patients prescribed to wear the same type and/or model of the wearable cardiac monitoring device 100 as the patient 104 (e.g., prescribed to wear a wearable cardiac monitoring device 100 that includes a garment versus a wearable cardiac monitoring device 100 that includes an adhesive patch and cardiac monitor, as described in further detail below). As another example, the predetermined cohort of other patients may be patients with similar incentive criteria as the patient 104 (e.g., having a predetermined number of incentive criteria in common with the patient 104, having incentive criteria within a certain range of the incentive criteria of the patient 104 such as within 10% or 20% of health goals for the patient 104, etc.). As another example, the predetermined cohort of other patients may be patients who share a demographic with the patient 104, such as are within the same age range, are the same gender, live in the same geographical area, and/or the like as the patient 104. As another example, the predetermined cohort of other patients may share a health status with the patient 104. To illustrate, the predetermined cohort of other patients and the patient 104 may have a same cardiac diagnosis, may have the same or similar comorbidities as the patient 104, may exhibit the same or similar symptoms as the patient 104 (e.g., chest pain, shortness of breath, fatigue, etc.), may be in the same stage of heart failure (e.g., as classified according to the AHA or NYHA heart failure stages), and/or so on. As another example, the predetermined cohort of other patients may have received a wearable cardiac monitoring device 100 to wear in the same date range as the patient 104 (e.g., within the same 3 day period, within the same 5 day period, within the same 7 day period, within the same 10 period, within the same 14 day period, etc.).

The remote server 102 provides an output indicating the progress of the patient 104 towards earning incentive rewards relative to the incentive rewards progress information of the predetermined cohort of other patients at step 312. In implementations, the output includes a visual representation indicating the progress of the patient 104 towards earning incentive rewards relative to the incentive rewards progress information of the predetermined cohort of the other ambulatory cardiac patients. For example, the visual representation may be configured to be displayed on a computing device, such as the clinician-authorized user terminal 106 and/or the patient user device 108. As such, the remote server 102 may be configured to transmit the visual representation to the clinician-authorized user terminal 106 and/or the patient user device 108. The visual representation may be configured to be displayed via, for instance, an application running on the computing device, via a browser running on the computing device (e.g., after the user of the computing device navigates to a webpage associated with the wearable cardiac monitoring and rehabilitative system and enters user credentials), an email opened on the computing device, a text or SMS message opened on the computing device, and/or the like. In some implementations, the output generated and transmitted to the clinician-authorized user terminal 106 may be the same as the output generated and transmitted to the patient user device 108, and in some implementations, the output generated and transmitted to the clinician-authorized user terminal 106 may be different from the output generated and transmitted to the patient user device 108. For instance, the output generated for the clinician-authorized user terminal 106 may include a dashboard showing information for all of the patients wearing wearable cardiac monitoring devices 100, including the patient 104, and how their incentive rewards progress information compares to each other. By contrast, the output generated for the patient user device 108 may include an initial dashboard showing the patient's progress towards earning incentive rewards and then illustrating how the patient's progress compares to the incentive rewards progress information of the predetermined cohort further down on the dashboard.

In implementations, the output includes information allowing the patient 104 to see the rewards they have earned. For example, the output may show the patient's incentive criteria, as well as rewards the patient 104 has earned and/or the patient's progress toward earning rewards for each incentive criterion. As another example, the output may show what rewards the patient 104 has earned, how the patient 104 earned the rewards, and when the patient 104 earned the rewards. To illustrate, the output may include a timeline that the patient 104 can scroll through to see when the patient 104 earned various rewards. As another example, the output may show a total amount of accrued rewards (e.g., at the top of a visual representation). As another example, the output may show milestones that the patient 104 has met with their earned rewards, such as milestones for total rewards earned, rewards earned in particular categories, etc.

In implementations, the output includes a variety of information about the predetermined cohort of other patients and how the patient's progress towards earning rewards compares to the progress of the predetermined cohort of other patients. As an example, the output may include basic community stats, such as how many people are in the predetermined cohort of other patients and how the patient 104 ranks in terms of rewards earned (e.g., in terms of total rewards earned, in terms of total rewards earned for different types of incentive criteria, etc.). In implementations, the output may include other types of statistics for the patient 104 and the predetermined cohort of other patients. Examples of other types of statistics may include a step count (e.g., daily step count, weekly step count, etc.), an activity level (e.g., minutes of physical activity completed per day, per week, etc.), an amount of time the wearable cardiac monitoring device 100 has been worn (e.g., per day, per week, etc.), a number of message acknowledgement clicks the patient has engaged in, a number of video views the patient has engaged in (e.g., views of educational videos about the wearable cardiac monitoring device 100, of videos aimed at improving the health of the patient, etc.), cumulative time the patient has watched videos, and/or the like.

In implementations, the output may include trend lines for the patient 104. The trend lines may show the amount of rewards the patient 104 has earned over time, progress the patient 104 has made towards achieving health goals (e.g., the patient's average resting heart rate over time, the patient's R-R interval over time, etc.), and/or the like. Additionally, the output may include how the trend lines for the patient 104 compare to similar trend lines for the predetermined cohort of other patients. For example, the output may include a trend of incentive rewards earned by the patient 104 (e.g., per day, per week, etc.) over a time period relative to a trend of incentive rewards earned by the predetermined cohort of other patients (e.g., the average amount of incentive rewards earned per day, per week, etc. by the predetermined cohort) over the same time period. In implementations, the remote server 102 is configured to adjust the output indicating the progress of the patient 104 towards earning incentive rewards relative to the incentive rewards progress information of the predetermined cohort of other patients. To illustrate, the remote server 102 may adjust the progress of patient 104 relative to the progress information of the predetermined cohort of other patients to compensate for differences in health between the patient 104 and the patients of the predetermined cohort. For example, if the patient 104 is less mobile than the average patient of the predetermined cohort, the remote server 102 may modify the milestones for the patient 104 to compensate for the decreased mobility of the patient 104. The remote server 102 may, for instance, lower the amount of rewards the patient 104 needs to earn to reach milestones, in total and/or for incentive criteria related to movement (e.g., physical activity and/or step counts incentive criteria). In examples, the remote server 102 may adjust the output by adjusting the amount of rewards that the patient 104 earns for completing various incentive criteria compared to the amount of rewards that the patients of the predetermined cohort earn for similar incentive criteria. As an illustration, if the patient 104 is less mobile than the average patient of the predetermined cohort, the remote server 102 may provide the patient 104 with comparatively more in rewards for performing less in physical activity compared to the average patient of the predetermined cohort.

In implementations, the remote server 102 may adjust the output indicating the ambulatory cardiac patient's progress towards earning incentive rewards relative to the incentive rewards progress information of the predetermined cohort of other patients to account for a health status of the patient 104 relative to the health statuses of the predetermined cohort of other ambulatory cardiac patients. As an illustration, the health status of the patient 104 may include a heart failure classification for the patient 104 and/or the amount of time that has elapsed since the patient 104 experienced a prior myocardial infarction or other serious cardiac event (if such event has occurred for the patient 104). Similarly, the health statuses of the predetermined cohort of other ambulatory cardiac patients may include the heart failure classifications for each of the other patients and/or the amount of time that has elapsed since each of the other patients experienced a prior myocardial infarction or other serious cardiac event (if such event has occurred for each of the other patients). The remote server 102 may then compensate the milestones, the rewards earned, etc. according to the health statuses of the patient 104 and the predetermined cohort of other patients to more equally reward the patient 104 and the predetermined cohort of other patients based on their respective ability to earn rewards.

In implementations, the remote server 102 may adjust the output indicating the ambulatory cardiac patient's progress towards earning incentive rewards relative to the incentive rewards progress information of the predetermined cohort of other ambulatory cardiac patients to account for at least one demographic of the patient 104 relative to the demographics of the predetermined cohort of other ambulatory cardiac patients. For instance, the at least one demographic of the patient 104 may include an age, gender, geographical location, whether the patient 104 is employed, and/or the like. Similarly, the demographics of the predetermine cohort of other ambulatory cardiac patients may include an age, gender, geographical location, whether the respective patient is employed, and/or the like for each patient of the predetermined cohort. The remote server 102 may then compensate the milestones, rewards earned, etc. for the patient 104 compared to the predetermined cohort of other patients based on a predicted ability to earn rewards from these demographics.

In implementations, the remote server 102 is configured to set an aggregate goal for a patient population including the patient 104 to meet. For example, the patient 104 and the predetermined cohort of other patients may together form a patient population. As another example, the patient 104 and a subset of the predetermined cohort of other patients may together form a patient population. As another example, the patient 104, the predetermined cohort of other patients, and additional patients may together form a patient population. As another example, all patients currently prescribed a wearable cardiac monitoring device 100 may together form a patient population.

Meeting the patient population's goal may provide all of the patients in the patient population with bonus rewards, may cause a donation to be made to a non-profit organization (e.g., a non-profit organization selected collectively by the patient population from a list, a non-profit organization related to heart health, etc.), and/or the like. As an illustration, the remote server 102 may receive aggregate incentive criteria for the patient population, where the aggregate incentive criteria define an aggregate goal for the patient population (e.g., using similar processes as described above with respect to step 202 of FIG. 2A and step 302 of FIG. 3). For example, the clinician for the whole patient population may submit aggregate incentive criteria to the remote server 102 that the remote server 102 uses, or clinicians for all of the patients in the patient population may each submit aggregate incentive criteria to the remote server 102 that the remote server 102 uses (e.g., by averaging the aggregate incentive criteria from the clinicians). The remote server 102 may then store, in a database of the remote server 102, an aggregate incentive data structure based on the aggregate incentive criteria (e.g., using similar processes as described above with respect to step 204 of FIG. 2A and step 304 of FIG. 3). The remote server 102 may update, based on ECG data for the patient population, motion data for the patient population, wear state data for the patient population, and/or other patient data for the patient population, the aggregate incentive data structure to indicate the patient population's progress towards earning incentive rewards (e.g., using similar processes as described above with respect to step 308 of FIG. 3). The remote server 102 may further provide an aggregate output indicating the patient population's progress towards completing the aggregate goal (e.g., using similar processes as described above with respect to step 312 of FIG. 3), which may be included, for example, in the individual outputs for the patients of the patient population.

In implementations, a patient population may be ranked against a different patient population, such as a concurrent population or a historical population. For example, a patient population may be formed of patients that received a wearable cardiac monitoring device 100 within the same time frame, such that multiple populations of patients wearing wearable cardiac monitoring devices 100 exist at the same time. The rewards progress of each population may be ranked against the respective rewards progress of the other concurrent and/or historical patient populations at the points when the patients of the other concurrent and/or historical patient populations had had their wearable cardiac monitoring devices 100 for the same amount of time or nearly the same amount of time as the present patient population. The ranking of the patient population may also provide the patient population with additional incentives or rewards. For example, for every week that the patient population remains within the top five patient populations, each patient of the patient population may receive bonus rewards. As another example, if the patient population is ranked first for a given week, a donation may be made to a non-profit organization in the name of the patient population.

In implementations, the patient 104 may need to opt into a community program before the patient 104 is provided with outputs showing the patient's progress towards earning incentive rewards compared to the incentive rewards progress information of a predetermined cohort of other patients. For example, the patient 104 may be provided with a message (e.g., via text, via email, via an application running on the patient user device 108, etc.) explaining the community program to the patient 104 and asking for the patient's consent to be included in the community program. If the remote server 102 receive an input from the patient 104 opting into the community program, the remote server 102 determines a cohort of other patients and provides the outputs indicating the patient's progress relative to the incentive rewards progress information of the predetermined cohort. If the remote server 102 does not receive an input from the patient 104 opting into the community program, the remote server 102 may provide the patient 104 with outputs that do not include the patient's progress relative to the incentive rewards progress information of a predetermined cohort. As an illustration, the patient 104 may only be able to view the rewards they have earned, partial progress towards earning rewards, timelines of earned rewards, trendlines of earned rewards, and/or the like on, e.g., a rewards dashboard (e.g., displayed to the patient via the patient user device 108).

In implementations, the patient 104 may be able to communicate with other patients prescribed to wear a wearable cardiac monitoring device 100. For example, the patient 104 may be able to access (e.g., via an application running on the patient user device 108, by logging into a community dashboard on a browser, etc.) a community message board available to all members of a patient population of which the patient 104 is a member. The patient 104 may be able to post messages, read messages, reply to messages, react to messages (e.g., post a “like” to a message), and/or so on. As another example, the patient 104 may be able to chat with other patients prescribed to wear a wearable cardiac monitoring device 100 (e.g., via an application running on the patient user device 108, by logging onto a community dashboard on a browser, etc.).

In implementations, the patient 104 may be able to share their rewards progress. For example, the patient 104 may be able to share their rewards progress with another patient prescribed to wear a wearable cardiac monitoring device 100, with a caregiver, with a loved one, and so on. For example, the patient 104 may be able to “friend” other patients on a dashboard (e.g., by sending a request to the other patient that the patient 104 wishes to friend and/or by accepting a request from the other patient) and request that the patient's progress be sent to the friends of the patient 104 (and, in examples, vice versa). As an illustration, the patient 104 may be able to send a request to the remote server 102 asking that the remote server 102 transmit their progress towards earning incentive rewards to a second patient who is part of patient's predetermined cohort of other ambulatory patients. In response to receiving the request, the remote server 102 may provide the progress of the patient 104 to the second patient. The remote server 102 may provide the progress of the patient 104 to the second patient on an ongoing basis (e.g., in a portion of a dashboard for showing the progress of “friends”), or the remote server 102 may provide a one-time snapshot of the progress of the patient 104 to the second patient.

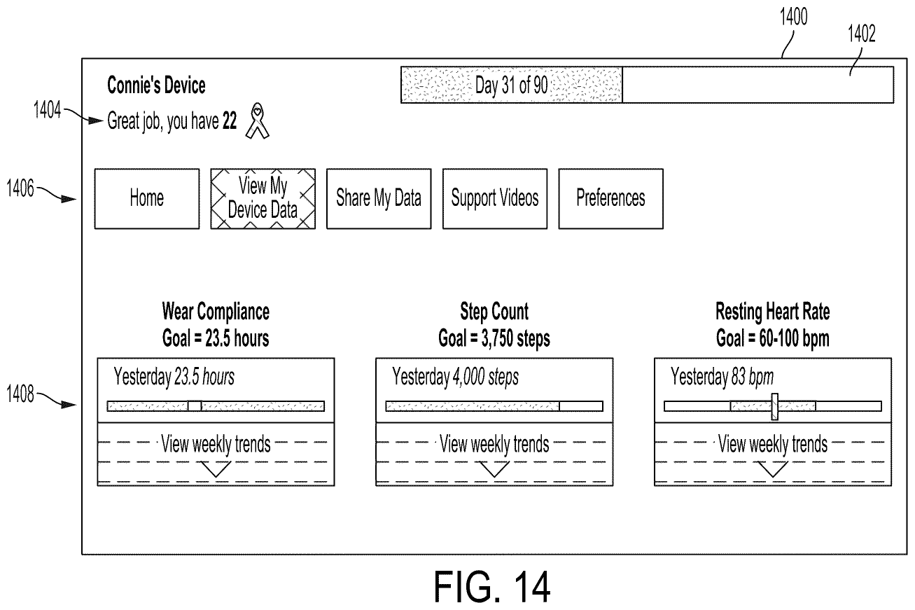

FIGS. 14-16 illustrate example user interfaces configured to display an output indicating the progress of the patient 104 towards earning incentive rewards. To begin with, FIG. 14 shows an example user interface 1400, as part of a patient engagement dashboard or portal, whereby the patient 104 can view their own incentive rewards progress information. The example user interface 1400 includes a prescription progress bar 1402 that illustrates the total length of the patient's prescription (e.g., 90 days in the example of FIG. 14) along with how many days the patient 104 has worn the wearable cardiac monitoring device 100 so far (e.g., 31 days in the example of FIG. 14). The user interface 1400 also includes a rewards summary 1404 showing the total number of incentive rewards the patient 104 has earned so far (e.g., 22 ribbons in the example of FIG. 14).

At the bottom of the user interface 1400, the patient 104 can view various incentive criteria and whether the patient 104 has been meeting these incentive criteria in an incentive criteria section 1408. For instance, as shown in the example of FIG. 14, the patient 104 has a wear compliance goal of 23.5 hours per day, a step count goal of 3,750 steps per day, and a resting heart rate goal of 60-100 bpm. The patient 104 can view whether they met these goals the previous day, and in the example of FIG. 14, they successfully met all three of these goals (e.g., as shown by the goals being in green in the incentive criteria section 1408). The patient 104 can also click on a drop-down button within the incentive criteria section 1408 to view weekly trends for each of these goals. For example, if the patient 104 clicks on the weekly trend button for wear compliance, the patient 104 may be shown a chart illustrating the average number of hours per day the patient 104 wore the wearable cardiac monitoring device 100 for each week of their prescription period. As another example, if the patient 104 clicks on the weekly trend button for wear compliance, the patient 104 may be shown a chart illustrating the average number of hours per day the patient 104 wore the wearable cardiac monitoring device 100 for each day of the present calendar week so far.

The example user interface 1400 also includes a menu 1406 with buttons that the patient 104 can press to view a “home” user interface, “device data” user interface (e.g., the user interface 1400 shown in FIG. 14), “sharing” user interface, “support” user interface, or “preferences” user interface. As an example, if the patient 104 clicks on the home button, the patient 104 may be shown a summary of their health and a list of tasks or activities to complete for the day (e.g., the incentive criteria the patient 104 is being encouraged to complete for the day). As another example, if the patient 104 clicks on the sharing data button, the patient 104 may be shown a user interface that they can use to enter in information about an individual the patient 104 would like to share their health and/or device 100 data with (e.g., a clinician, a caregiver for the patient 104, a member of the patient's predetermined cohort, etc.). As another example, if the patient 104 clicks on the support button, the patient 104 may be shown a list of frequently asked questions about the patient engagement program portal and/or the wearable cardiac monitoring device 100, as well as contact information in case the patient 104 experiences issues with the portal and/or device 100. As another example, if the patient 104 clicks on the preferences button, the patient 104 may be shown a list of settings or preferences that the patient 104 can modify related to the patient engagement program portal, such as settings for who can view the patient's health, device 100, and rewards information shown to the patient 104 on the portal.

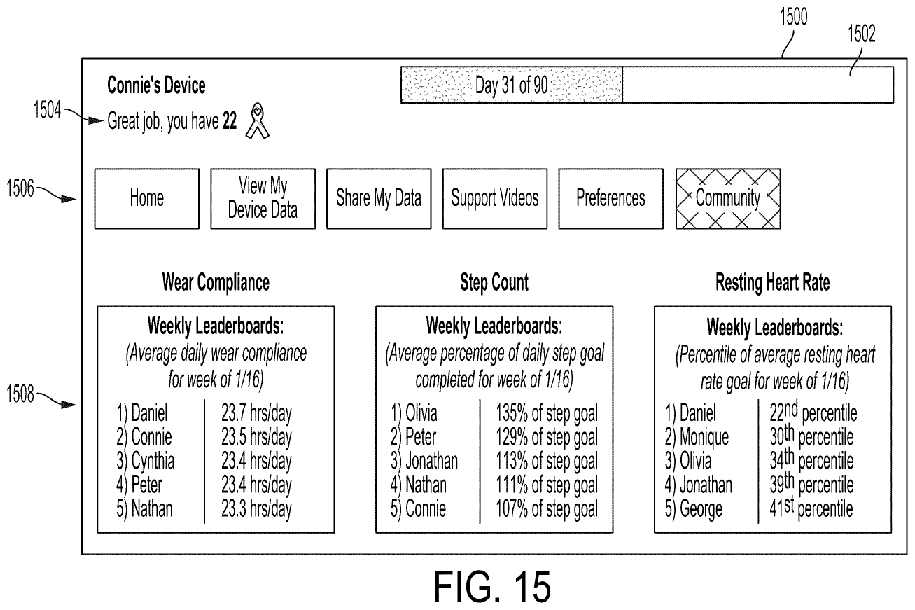

Referring now to FIG. 15, FIG. 15 shows another example user interface 1500 incorporated as part of a patient engagement dashboard or portal. The user interface 1500 is configured similarly to the user interface 1400 of FIG. 14, including a prescription progress bar 1502, a rewards summary 1504, and a menu 1506. However, the menu 1506 of the user interface 1500 also includes a “community” button that is currently selected. The user interface 1500 thus includes a community section 1508 that illustrates the patient's progress towards earning incentive rewards compared to the incentive rewards progress information of the patient's predetermined cohort. In the example of FIG. 15, the community section 1508 includes a weekly leaderboard for each of the patient's incentive criteria shown on the user interface 1400 of FIG. 14: wear compliance, step count, and resting heart rate. The weekly leaderboard for each of these incentive criteria ranks the patients of the predetermined cohort, including the patient 104, according to how well they met their incentive criteria for the previous week.

For instance, as shown in FIG. 15, the weekly leaderboard for wear compliance shows, on average, how many hours per day the cohort leaders wore the wearable cardiac monitoring device 100 per day for the previous calendar week. The weekly leaderboard for step count shows, on average, what percentage of their step count goal the cohort leaders completed per day for the previous calendar week. The weekly leaderboard for resting heart rate shows what percentile, on average, the cohort leaders placed within their respective resting heart rate goal ranges over the course of the previous calendar week. In the example of FIG. 15, each patient of the predetermined cohort may thus have the same wear compliance goal (e.g., 23.5 hours per day, as shown in the user interface 1400) but may have different step count and resting heart rate goals. As such, ranking the patients of the predetermined cohort according to step count goal percentage and resting heart rate goal percentile may normalize the patients' progress compared to each other.

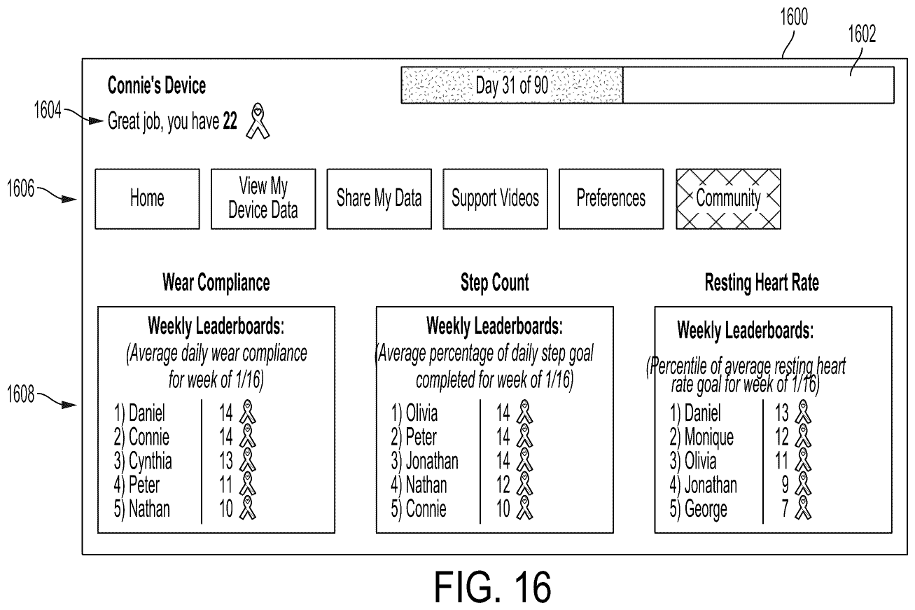

Referring now to FIG. 16, FIG. 16 shows another example user interface 1600 incorporated as part of a patient engagement dashboard or portal. The user interface 1600 is configured similarly to the user interface 1400 of FIG. 14 and the user interface 1500 of FIG. 15, including a prescription progress bar 1602, a rewards summary 1604, and a menu 1606. The user interface 1600 also includes a community section 1608 configured similarly to the community section 1508 of FIG. 15. However, instead of the weekly leaderboards showing the actual progress of the cohort leaders towards achieving their incentive criteria, the weekly leaderboards shown in the community section 1608 show the number of ribbons the cohort leaders have earned for meeting wear compliance, step count, and resting heart rate goals. In implementations, the community section 1608 may additionally or alternatively display the total number of ribbons cohort leaders earned over the previous week. Additionally, the user interfaces 1400, 1500, and 1600 are intended to be examples. Other user interfaces showing the patient's progress towards earning incentive rewards and/or the progress of other patient's in the patient's predetermined cohort towards earning incentive rewards (e.g., including additional, less, and/or alternative information) may be displayed to the patient 104.

Returning to the wearable cardiac monitoring device 100, FIG. 4 shows the wearable cardiac monitoring device 100 according to some implementations. The wearable cardiac monitoring device 100 shown in FIG. 4 is external and wearable by the patient 104 around the patient's torso. Such a wearable cardiac monitoring device 100 can be, for example, capable of and designed for moving with the patient 104 as the patient 104 goes about their daily routine. For example, the wearable cardiac monitoring device 100 as described herein with respect to FIGS. 4 and 5 can be a wearable cardioverter defibrillator, configured to be bodily-attached to the patient 104. In one example scenario, such wearable defibrillators can be worn nearly continuously or substantially continuously for a week, two weeks, a month, or two or three months at a time. During the period of time in which they are worn by the patient 104, the wearable defibrillators can be configured to continuously or substantially continuously monitor the vital signs of the patient 104 and, upon determination that treatment is required, can be configured to deliver one or more therapeutic electrical pulses to the patient 104. For example, such therapeutic shocks can be pacing, defibrillation, cardioversion, or transcutaneous electrical nerve stimulation (TENS) pulses.

The wearable cardiac monitoring device 100 can include one or more of the following: a garment 400 configured to be worn about the patient's torso; one or more externally worn sensors configured to output one or more physiological signals, such as electrodes 402 (e.g., ECG electrodes configured to output at least on ECG signal); one or more therapy electrodes 404a and 404b (collectively referred to herein as therapy electrodes 404); a medical device controller 406; a connection pod 408; a patient interface pod 410; a belt 412; or any combination of these. In implementations, the one or more externally worn sensors may include additional sensors, such as one or more motion detectors configured to generate motion data indicative of physical activity performed by the patient 104, one or more wear state sensors configured to detect a wear state of the wearable cardiac monitoring device 100, one or more bioacoustics sensors configured to generate bioacoustics signals for the heart of the patient 104, one or more respiration sensors configured to generate respiration signals indicative of the respiration activity of the patient 104, and/or the like. In some examples, at least some of the components of the wearable cardiac monitoring device 100 can be configured to be mounted on or affixed to the garment 400, such as by mating hooks, hook-and-loop fabric strips, receptacles (e.g., pockets), and the like. For instance, the sensing electrodes 402 may be mounted on the garment 400 by hook-and-look fabric strips on the electrodes 402 and the garment 400, and the therapy electrodes 404 may be mounted on the garment 400 by being inserted into receptacles of the garment 400. In some examples, at least some of the components of the wearable cardiac monitoring device 100 can be permanently integrated into the garment 400, such as by being sewn into the garment. In some examples, at least some of the components may be connected to each other through cables, through sewn-in connections (e.g., wires woven into the fabric of the garment 400), through conductive fabric of the garment 400, and/or the like.

The medical device controller 406 can be operatively coupled to the sensing electrodes 402, which can be affixed to the garment 400 (e.g., assembled into the garment 400 or removably attached to the garment 400, for example, using hook-and-look fasteners). In some implementations, the sensing electrodes 402 can be permanently integrated into the garment 400. The medical device controller 406 can also be operatively coupled to the therapy electrodes 404. For example, the therapy electrodes 404 can also be assembled into the garment 400, or, in some implementations, the therapy electrodes 404 can be permanently integrated into the garment 400. As shown in FIG. 4, the sensing electrodes 402 and/or the therapy electrodes 404 can be directly operatively coupled to the medical device controller 406 and/or operatively coupled to the medical device controller 406 through the connection pod 408. Component configurations other than those shown in FIG. 4 are also possible. For example, the sensing electrodes 402 can be configured to be attached at various positions about the body of the patient 104. In some implementations, the sensing electrodes 402 and/or at least one of the therapy electrodes 404 can be included on a single integrated patch and adhesively applied to the patient's body. In some implementations, the sensing electrodes 402 and/or at least one of the therapy electrodes 404 can be included in multiple patches and adhesively applied to the patient's body. Such patches may be in a wired (e.g., via the connection pod 408) or wireless connection with the medical device controller 406.

The sensing electrodes 402 can be configured to detect one or more cardiac signals. Examples of such signals include ECG signals and/or sensed cardiac physiological signals from the patient 104. Example sensing electrodes 402 include a metal electrode with an oxide coating such as tantalum pentoxide electrodes. For example, by design, digital sensing electrodes can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In some examples, the electrodes 402 can be used with an electrolytic gel dispersed between the electrode surface and the patient's skin. In certain implementations, the sensing electrodes 402 can be dry electrodes that do not need an electrolytic material. As an example, such a dry electrode can be based on tantalum metal and having a tantalum pentoxide coating as is described above. Such dry electrodes can be more comfortable for long-term monitoring applications.

In implementations, the sensing electrodes 402 can include additional components such as accelerometers, acoustic signal detecting devices, biovibrational sensors, and other measuring devices for recording additional parameters. For example, the sensing electrodes 402 can also be configured to detect other types of patient physiological parameters and acoustic signals, such as tissue fluid levels, heart vibrations, lung vibrations, respiration vibrations, patient movement, etc. In implementations, the wearable cardiac monitoring device 100 may include sensors or detectors separate from the sensing electrodes 402, such as separate motion detector(s), wear state detector(s), bioacoustics sensor(s), biovibrational sensor(s), respiration sensor(s), temperature sensor(s), pressure sensor(s), and/or the like.

In some examples, the therapy electrodes 404 can also be configured to include sensors configured to detect ECG signals as well as, or in the alternative, other physiological signals from the patient 104. The connection pod 408 can, in some examples, include a signal processor configured to amplify, filter, and digitize these cardiac signals prior to transmitting the cardiac signals to the medical device controller 406. One or more therapy electrodes 404 can be configured to deliver one or more therapeutic cardioversion/defibrillation shocks to the body of the patient 104 when the wearable cardiac monitoring device 100 determines that such treatment is warranted based on the signals detected by the sensing electrodes 402 and processed by the medical device controller 406. Example therapy electrodes 404 can include conductive metal electrodes such as stainless-steel electrodes that include, in certain implementations, one or more conductive gel deployment devices configured to deliver conductive gel between the metal electrode and the patient's skin prior to delivery of a therapeutic shock.

In some implementations, a wearable cardiac monitoring device 100 as described herein can be configured to switch between a therapeutic mode and a monitoring mode such that the wearable cardiac monitoring device 100 is configured to only monitor a patient (e.g., not provide or perform any therapeutic functions). For example, in such implementations, therapeutic components such as the therapy electrodes 404 and associated circuitry may be decoupled from (or coupled to) or switch out of (or switched into) the wearable cardiac monitoring device 100. As an illustration, a wearable cardiac monitoring device 100 can have optional therapeutic elements (e.g., defibrillation and/or pacing electrode components and associated circuitry) that are configured to operate in a therapeutic mode. The optional therapeutic elements may be physically decoupled from the wearable cardiac monitoring device 100 as a means to convert the device 100 from a therapeutic mode into a monitoring mode. Alternatively, the optional therapeutic elements may be deactivated (e.g., by means of a physical or software switch), essentially rendering the wearable cardiac monitoring device 100 as a monitoring-only device for a specific physiological purpose for a particular patient. As an example of a software switch, an authorized person may be able to access a protected user interface of the wearable cardiac monitoring device 100 and select a preconfigured option or perform some other user action via the user interface to deactivate the therapeutic elements of the wearable cardiac monitoring device 100.

FIG. 5A illustrates a sample component-level view of a medical device controller 501 included in a wearable cardiac monitoring device 100. The medical device controller 501 is an example of the controller 406 shown in FIG. 4 and described above. As shown in FIG. 5A, the medical device controller 501 can include a housing 517 configured to house a therapy delivery circuit 500 configured to provide one or more therapeutic shocks to the patient 104 via the therapy electrodes 404, a data storage 502, a network interface 504, a user interface 506, at least one battery 508 (e.g., within a battery chamber configured for such purpose), a sensor interface 510 (e.g., to interface with the sensing electrodes 402 and other physiological sensors or detectors such as vibrational sensors, lung fluid sensors, infrared and near-infrared-based pulse oxygen sensors, and blood pressure sensors, among others), a cardiac event detector 514, an alarm manager 524, and at least one processor 516. As described above, in some implementations, the wearable cardiac monitoring device 100 that includes like components as those described above but does not include the therapy delivery circuit 500 and the therapy electrodes 404 (shown in dotted lines). That is, in some implementations, the wearable cardiac monitoring device 100 can include the ECG monitoring components and not provide therapy to the patient.

The therapy delivery circuit 500 can be coupled to the therapy electrodes 404 configured to provide therapy to the patient 104. For example, the therapy delivery circuit 500 can include, or be operably connected to, circuitry components that are configured to generate and provide an electrical therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit 500 and under the control of one or more processors (e.g., processor 516) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation.

The capacitors can include a parallel-connected capacitor bank consisting of a plurality of capacitors (e.g., two, three, four, or more capacitors). In some examples, the capacitors can include a single film or electrolytic capacitor as a series connected device including a bank of the same capacitors. These capacitors can be switched into a series connection during discharge for a defibrillation pulse. For example, four capacitors of approximately 140 uF or larger, or four capacitors of approximately 650 uF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 500 can be configured to perform the switching and pulse delivery operations, e.g., under control of the processor 516. As the energy is delivered to the patient 104, the amount of energy being delivered can be tracked. For example, the amount of energy can be kept to a predetermined constant value even as the pulse waveform is dynamically controlled based on factors, such as the patient's body impedance, while the pulse is being delivered. In certain examples, the therapy delivery circuit 500 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

The data storage 502 can include one or more of non-transitory computer readable media, such as flash memory, solid state memory, magnetic memory, optical memory, cache memory, combinations thereof, and others. The data storage 502 can be configured to store executable instructions and data used for operation of the medical device controller 501. In some implementations, the data storage 502 can include sequences of executable instructions that, when executed, are configured to cause the processor 516 to perform one or more functions. For example, the data storage 502 can be configured to store information such as ECG data as received from, for instance, the sensor interface 510.

In some examples, the network interface 504 can facilitate the communication of information between the medical device controller 406 and one or more devices or entities over a communications network. For example, the network interface 504 can be configured to communicate with the remote server 102 or other similar computing device. The network interface 504 can include communications circuitry for transmitting data in accordance with a Bluetooth® wireless standard for exchanging such data over short distances to an intermediary device(s) (e.g., abase station, “hotspot” device, smartphone, tablet, portable computing device, and/or other device in proximity with the wearable cardiac monitoring device 100). The intermediary device(s) may in turn communicate the data to the remote server 102 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 102 over a Wi-Fi communications link based on the IEEE 802.11 standard. In some implementations, the network interface 504 may be configured to instead communicate directly with the remote server 102 without the use of intermediary device(s). In such implementations, the network interface 504 may use any of the communications links and/or protocols provided above.

In some implementations, the user interface 506 may include one or more physical interface devices, such as input devices, output devices, and combination input/output devices, and a software stack configured to drive operation of the devices. These user interface elements may render visual, audio, and/or tactile content. Thus, the user interface 506 may receive inputs and/or provide outputs, thereby enabling a user to interact with the medical device controller 406. In some implementations, the user interface 506 may include the patient user device 108, while in some implementations, the user interface 506 may include a device separate from the patient user device 108.

The medical device controller 406 can also include at least one battery 508 configured to provide power to one or more components integrated in the medical device controller 406. The battery 508 can include a rechargeable multi-cell battery pack. In one example implementation, the battery 508 can include three or more cells (e.g., 2200 mA lithium ion cells) that provide electrical power to the other device components within the medical device controller 406. For example, the battery 508 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 406.

The sensor interface 510 can include physiological signal circuitry that is coupled to one or more externally worn sensors configured to monitor one or more physiological parameters of the patient and output one or more physiological signals. As shown, the sensors may be coupled to the medical device controller 501 via a wired or wireless connection. The sensors can include one or more sensing electrodes 402 (e.g., ECG electrodes) configured to output at least one ECG signal. In some implementations, the sensors can include conventional ECG sensing electrodes and/or digital sensing electrodes. The sensors can also include one or more non-ECG physiological sensors 520 such as one or more vibration sensors 518, tissue fluid monitors 526 (e.g., based on ultra-wide band RF devices), one or more motion sensors (e.g., accelerometers, gyroscopes, and/or magnetometers), a temperature sensor, a pressure sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

The one or more vibration sensors 518 can be configured to detect cardiac or pulmonary vibration information. For example, the vibration sensors 518 can detect a patient's heart valve vibration information. For example, the vibration sensors 518 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensors 518 can also be configured to detect heart wall motion, for instance, by placement of the sensor in the region of the apical beat. The vibration sensors 518 can include a vibrational sensor configured to detect vibrations from a patient's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on sounds produced within the lungs (e.g., stridor, crackle, etc.). The vibration sensors 518 can also include a multi-channel accelerometer, for example, a three-channel accelerometer configured to sense movement in each of three orthogonal axes such that patient movement/body position can be detected and correlated to detected cardio-vibrations information. The vibration sensors 518 can transmit information descriptive of the cardio-vibrations information to the sensor interface 510 for subsequent analysis.

The tissue fluid monitors 526 can use RF based techniques to assess fluid levels and accumulation in a patient's body tissue. For example, the tissue fluid monitors 526 can be configured to measure fluid content in the lungs, typically for diagnosis and follow-up of pulmonary edema or lung congestion in heart failure patients. The tissue fluid monitors 526 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 526 can transmit information descriptive of the tissue fluid levels to the sensor interface 510 for subsequent analysis.

The controller 501 can further include a motion detector interface operably coupled to one or more motion detectors configured to generate motion data, for example, indicative of physical activity performed by the patient 104. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. As an illustration, the motion data may include accelerometer counts indicative of physical activity, accelerometer counts indicative of respiration rate, and posture information for the patient 104. For instance, in some implementations, the controller 501 can include an accelerometer interface 512 operably coupled to one or more accelerometers 522, as shown in FIG. 5A. Alternatively, in some implementations, the accelerometer interface 512 may be incorporated into other components of the controller 501. As an example, the accelerometer interface 512 may be part of the sensor interface 510, and the one or more accelerometers 522 may be part of the non-ECG physiological sensors 520.

The accelerometer interface 512 is configured to receive one or more outputs from the accelerometers. The accelerometer interface 512 can be further configured to condition the output signals by, for example, converting analog accelerometer signals to digital signals (if using an analog accelerometer), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In some examples, the accelerometer interface 512 can be configured to filter the signals using a high-pass or band-pass filter to isolate the acceleration of the patient due to movement from the component of the acceleration due to gravity.

Additionally, the accelerometer interface 512 can configure the output for further processing. For example, the accelerometer interface 512 can be configured to arrange the output of an individual accelerometer 522 as a vector expressing the acceleration components of the x-axis, the y-axis, and the z-axis as received from each accelerometer. The accelerometer interface 512 can be operably coupled to the processor 516 and configured to transfer the output signals from the accelerometers 522 to the processor for further processing and analysis.

The one or more accelerometers 522 can be integrated into one or more components of the wearable cardiac monitoring device 100. In some implementations, the one or more motion detectors 522 may be located in or near the sensing electrodes 402. In some implementations, the one or more motion detectors 522 may be located elsewhere on the wearable cardiac monitoring device 100. For example, a motion detector 522 can be integrated into the controller 501 (e.g., such that the one or more motion detectors 522 would be located within the block diagram for the medical device controller 501 shown in FIG. 5A). In some examples, a motion detector 522 can be integrated into one or more of a therapy electrode 404, a sensing electrode 402, the connection pod 408, and/or into other components of the wearable cardiac monitoring device 100. In some examples, a motion detector 522 can be integrated into an adhesive ECG sensing and/or therapy electrode patch.

As described above, the sensor interface 510 and the accelerometer interface 512 can be coupled to any one or combination of sensing electrodes/other sensors to receive patient data indicative of patient parameters. Once data from the sensors has been received by the sensor interface 510 and/or the accelerometer interface 512, the data can be directed by the processor 516 to an appropriate component within the medical device controller 501. For example, ECG signals collected by the sensing electrodes 402 may be transmitted to the sensor interface 510, and the sensor interface 510 can transmit the ECG signals to the processor 516, which, in turn, relays the data to the cardiac event detector 514. The sensor data can also be stored in the data storage 502 and/or transmitted to the remote server 102 via the network interface 504. For instance, the processor 516 may transfer the ECG signals from the sensing electrodes 402 and the motion data from the one or more accelerometers 522 to the remote server 102.

In implementations, the cardiac event detector 514 can be configured to monitor the patient's ECG signal for an occurrence of a cardiac event such as an arrhythmia or other similar cardiac event. The cardiac event detector can be configured to operate in concert with the processor 516 to execute one or more methods that process received ECG signals from, for example, the sensing electrodes 402 and determine the likelihood that a patient is experiencing a cardiac event, such as a treatable arrhythmia. The cardiac event detector 514 can be implemented using hardware or a combination of hardware and software. For instance, in some examples, cardiac event detector 514 can be implemented as a software component that is stored within the data storage 502 and executed by the processor 516. In this example, the instructions included in the cardiac event detector 514 can cause the processor 516 to perform one or more methods for analyzing a received ECG signal to determine whether an adverse cardiac event is occurring, such as a treatable arrhythmia. In other examples, the cardiac event detector 514 can be an application-specific integrated circuit (ASIC) that is coupled to the processor 516 and configured to monitor ECG signals for adverse cardiac event occurrences. Thus, examples of the cardiac event detector 514 are not limited to a particular hardware or software implementation.

In response to the cardiac event detector 514 determining that the patient 104 is experiencing a treatable arrhythmia, the processor 516 is configured to deliver a cardioversion/defibrillation shock to the patient 104 via the therapy electrodes 404. In some implementations, the alarm manager 524 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 102 or may be implemented as one or more separate systems). For example, when the processor 516 determines using data from the sensing electrodes 402 that the patient is experiencing a treatable arrhythmia, the alarm manager 524 may issue an alarm via the user interface 506 that the patient is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient that the patient can abort the delivery of the defibrillating shock by interacting with the user interface 506. For instance, the patient may be able to press a user response button or user response buttons on the user interface 506, after which the alarm manager 524 will cease issuing an alert and the medical device controller 406 will no longer prepare to deliver the defibrillating shock.

The alarm manager 524 can be implemented using hardware or a combination of hardware and software. For instance, in some examples, the alarm manager 524 can be implemented as a software component that is stored within the data storage 502 and executed by the processor 516. In this example, the instructions included in the alarm manager 524 can cause the processor 516 to configure alarm profiles and notify intended recipients using the alarm profiles. In other examples, the alarm manager 524 can be an application-specific integrated circuit (ASIC) that is coupled to the processor 516 and configured to manage alarm profiles and notify intended recipients using alarms specified within the alarm profiles. Thus, examples of the alarm manager 524 are not limited to a particular hardware or software implementation.

In some implementations, the processor 516 includes one or more processors (or one or more processor cores) that each are configured to perform a series of instructions that result in the manipulation of data and/or the control of the operation of the other components of the medical device controller 501. In some implementations, when executing a specific process (e.g., cardiac monitoring), the processor 516 can be configured to make specific logic-based determinations based on input data received. The processor 516 may be further configured to provide one or more outputs that can be used to control or otherwise inform subsequent processing to be carried out by the processor 516 and/or other processors or circuitry with which the processor 516 is communicably coupled. Thus, the processor 516 reacts to a specific input stimulus in a specific way and generates a corresponding output based on that input stimulus. In some example cases, the processor 516 can proceed through a sequence of logical transitions in which various internal register states and/or other bit cell states internal or external to the processor 516 may be set to logic high or logic low.

As referred to herein, the processor 516 can be configured to execute a function where software is stored in a data store (e.g., the data storage 502) coupled to the processor 516, the software being configured to cause the processor 516 to proceed through a sequence of various logic decisions that result in the function being executed. The various components that are described herein as being executable by the processor 516 can be implemented in various forms of specialized hardware, software, or a combination thereof. For example, the processor 516 can be a digital signal processor (DSP) such as a 24-bit DSP processor. As another example, the processor 516 can be a multi-core processor, e.g., having two or more processing cores. As another example, the processor 516 can be an Advanced RISC Machine (ARM) processor, such as a 32-bit ARM processor. The processor 516 can execute an embedded operating system and further execute services provided by the operating system, where these services can be used for file system manipulation, display and audio generation, basic networking, firewalling, data encryption, communications, and/or the like.

As noted above, a wearable cardiac monitoring device, such as the wearable cardiac monitoring device 100, can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device such as processor 516 of the controller 501 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

The wearable cardiac monitoring device 100 is configured for long-term and/or extended use or wear by, or attachment or connection to, a patient. For example, devices as described herein may be capable of being continuously used or continuously worn by, or attached or connected to a patient, without substantial interruption (e.g., up to 24 hours or beyond, such as for weeks, months, or even years). In some implementations, such devices may be removed for a period of time before use, wear, attachment, or connection to the patient is resumed. As an illustration, devices may be removed to change batteries, carry out technical service, update the device software or firmware, and/or to take a shower or engage in other activities, without departing from the scope of the examples described herein. Such substantially or nearly continuous use or wear as described herein may nonetheless be considered continuous use or wear. Additionally, the wearable cardiac monitoring device 100 may be configured to transmit signals and data to the remote server 102 continuously. For example, the wearable cardiac monitoring device 100 shown and described with respect to FIGS. 4 and 5A above may be configured to transmit signals and data to the remote server 102 continuously similarly to the processes described below with respect to the cardiac monitor 600 and the portable gateway 604.

As described herein, and noted above, implementations of the present disclosure include monitoring medical device wear compliance for the patient 104. More specifically, the wear compliance information includes an accurate overview of what portion or percentage of a certain time period the patient has worn the wearable cardiac monitoring device 100 and how this compares to the expected wear for the patient 104 as prescribed, for example, by their clinician or other healthcare provider when being prescribed the wearable cardiac monitoring device 100. FIG. 5B illustrates an example reduced component-level view of the medical device controller 501 that includes the processor 516 that is configured to monitor wear compliance information for the patient 104 as described herein. For example, the processor 516 can include wear time circuitry, such as a wear compliance detector 530 as shown in FIG. 5B. The wear compliance detector 530 may be integrated into the processor 516 as illustrated in FIG. 5B, or the wear compliance detector 530 may be integrated as a separate processing component operably coupled to the processor 516. The wear compliance detector 530 can be implemented as a dedicated microprocessor and associated circuitry disposed on a printed circuit board (PCB) along with other components as described herein. The wear compliance detector 530, when implemented in a dedicated microprocessor or integrated into the processor 516, can be based on a series of processor-readable instructions configured to be executed by the dedicated microprocessor or processor 516. For example, the instructions can be implemented in a programming language such as C, C++, assembly language, machine code, HDL, or VHDL. In examples, the dedicated microprocessor can be an Intel-based microprocessor such as an X86 microprocessor or a Motorola 68020 microprocessor, each of which can use a different set of binary codes and/or instructions for similar functions. In implementations, the dedicated microprocessor or processor 516 can be configured to implement wear onset event detection and wear offset event detection as set forth in FIG. 2B above.

As further shown in FIG. 5B, the wear compliance detector 530 can include an onset event detector 532 and an offset event detector 534. As described above, the wear compliance detector 530 can be a dedicated microprocessor and associated circuitry disposed on a PCB along with other components as described herein. In implementations, a first microprocessor can be implemented as the onset event detector 532, and a second microprocessor can be implemented as the offset event detector 534. In some implementations, both the onset event detector 532 and offset event detector 534 can be implemented in the same microprocessor as described above. The onset event detector 532 and/or offset event detector 534, when implemented in a dedicated microprocessor or integrated into the processor 516, can be based on a series of processor-readable instructions configured to be executed by the dedicated microprocessor or processor 516.

As noted above, when a patient puts on the wearable cardiac monitoring device 100, a wear onset event can be determined based upon analysis of signals received from one or more of the sensors described herein. For example, based upon monitoring of signals output by the sensing electrodes 402 as well as signals output by the accelerometers 522, the onset event detector 532 can determine an onset event indicative of the patient 104 putting on or otherwise wearing the wearable cardiac monitoring device 100. Similarly, the offset event detector 534 can determine an offset event indicative of the patient 104 turning off, removing, or otherwise stopping the wearable cardiac monitoring device 100 from monitoring. Based upon the measured onset and offset events, the wear compliance detector 530 and/or the processor 516 can determine wear compliance information (e.g., wear time) for the patient 104.

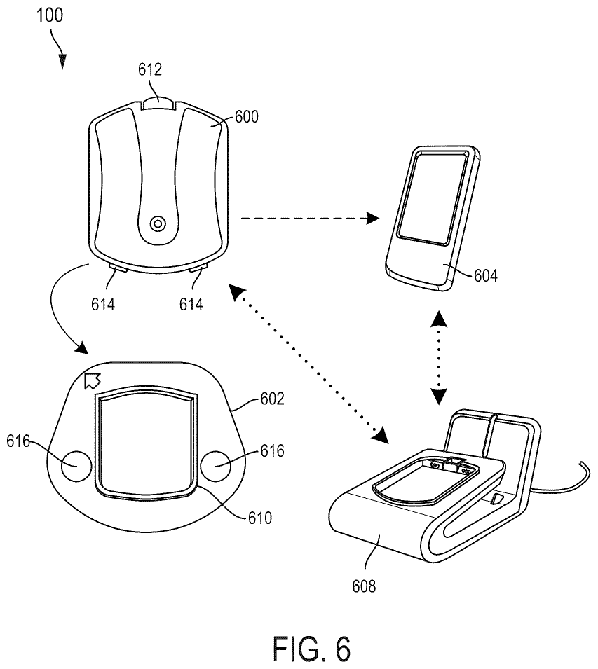

FIG. 6 shows the wearable cardiac monitoring device 100 according to another implementation. The wearable cardiac monitoring device 100 shown in FIG. 6 includes a cardiac monitor 600, an adhesive patch 602, a portable gateway 604, and a charger 608. The cardiac monitor 600 is configured to monitor, for example, cardiac arrhythmias of the patient 104, motion of the patient 104, lung fluid levels of the patient 104, heart sounds of the patient 104, etc. Accordingly, the cardiac monitor 600 is configured to gather signals from the patient 104 and transmit those signals to the remote server 102. For example, the cardiac monitor 600 may sense signals from the patient 104 (e.g., ECG signals) and acquire motion signals from the patient 104 (e.g., 1-axis channel, 2-axis channel, 3-axis channel, or multi-axis channel accelerometer signals, gyroscope signals, magnetometer signals, ballistocardiograph signals, and the like) and transmit the ECG and motion signals to the portable gateway 604. In turn, the portable gateway 604 transmits the sensed and acquired signals to the remote server 102. Alternatively, in some embodiments, the wearable cardiac monitoring device 100 shown in FIG. 6 does not include a portable gateway 604 and instead includes the cardiac monitor 600 (e.g., with internal communications/network circuitry) and the charger 608.

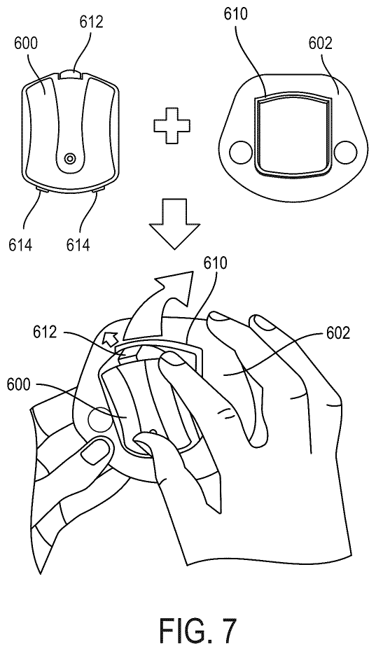

In embodiments, the adhesive patch 602 is configured to be adhesively coupled to the skin of the patient 104 such that the adhesive patch 602 can be worn on the skin of the patient 104 for an extended period of time. The adhesive patch 602 is further configured such that the cardiac monitor 600 may be removably attached (e.g., mounted) to the adhesive patch 602. The adhesive patch 602 may be disposable (e.g., single- or few-use patches) and may be made of a biocompatible, non-woven material. Additionally, the adhesive patch 602 may include a patch frame 610 delineating the boundary of the region of the patch 602 that is configured to house the cardiac monitor 600. In some embodiments, the cardiac monitor 600 may be designed for long-term usage. In such embodiments, the connection between the adhesive patch 602 and the cardiac monitor 600 may be configured to be reversible, e.g., the cardiac monitor 600 may be configured to be removably attached to the adhesive patch 602. For example, as shown in FIG. 6, the cardiac monitor 600 may include components such as one or more snap-in clips 612 that are configured to secure the cardiac monitor 600 to the adhesive patch 602 upon attachment to the patch frame 610. After the cardiac monitor 600 is attached to the patch frame 610, for instance, a user may press the snap-in clip 612 to subsequently release the cardiac monitor 600 from the patch frame 610. In some implementations, the cardiac monitor 600 may also include positioning tabs 614 that facilitate the attachment process between the cardiac monitor 600 and the adhesive patch 602. For example, the positioning tabs 614 may guide a user to insert the cardiac monitor 600 onto the correct portion of the patch frame 610 such that the cardiac monitor 600 can then be coupled, connected, or snapped into the patch frame 610 using the snap-in clip 612, as shown in FIG. 7.

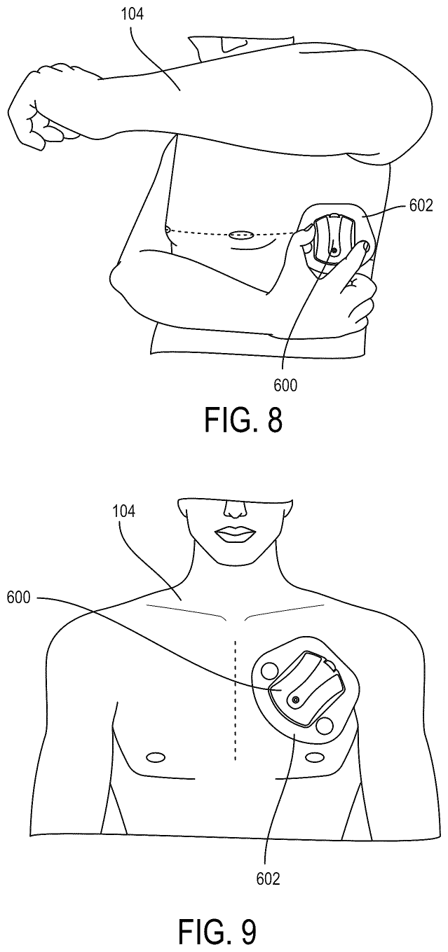

FIGS. 8 and 9 show examples of locations on the body of a patient 104 where an adhesive patch 602 housing a cardiac monitor 600 may be placed. As an example, the adhesive patch 602 may be placed on the side of the patient's thorax (e.g., under the patient's armpit, level with a circumferential line running across the patient's chest), as illustrated in FIG. 7. As another example, the adhesive patch 602 may be placed on the front of the patient's thorax (e.g., to the left of a medial line running between the patient's collarbone, for patients without dextrocardia), as illustrated in FIG. 8. However, the adhesive patch 602 may also be placed on any part of the patient's thorax that allows for efficient monitoring and recording of physiological data that includes RF waves received from the patient's thorax and containing information about the patient's thoracic fluid level.

In some implementations, the adhesive patch 602 may be designed to maintain attachment to skin of the patient 104 for several days (e.g., in a range from about 4 days to about 10 days, from about 3 days to about 5 days, from about 5 days to about 7 days, from about 7 days to about 10 days, from about 10 days to about 14 days, from about 14 days to about 30 days, etc.). After the period of use, the adhesive patch 602 can be removed from the patient's skin and the cardiac monitor 600 can be removed from the adhesive patch 602. The cardiac monitor 600 can be removably coupled, connected, or snapped onto a new adhesive patch 602 and reapplied to the patient's skin.

The cardiac monitor 600 and adhesive patch 602 are configured for long-term and/or extended use or wear by, or attachment or connection to, a patient. For example, devices as described herein are capable of being continuously used or continuously worn by, or attached or connected to, a patient without substantial interruption (e.g., for 24 hours, 2 days, 5 days, 7 days, 2 weeks, 30 days or 1 month, or beyond such as multiple months or even years). In some implementations, such devices may be removed for a period of time before use, wear, attachment, or connection to the patient is resumed. As an illustration, the cardiac monitor 600 may be removed for charging, to carry out technical service, to update the device software or firmware, for the patient to take a shower, and/or for other reasons or activities without departing from the scope of the examples described herein. As another illustration, the patient may remove a used adhesive patch 602, as well as the cardiac monitor 600, so that the patient may adhere a new adhesive patch 602 to their body and attach the cardiac monitor 600 to the new adhesive patch 602. Such substantially or nearly continuous use, monitoring, or wear as described herein may nonetheless be considered continuous use, monitoring, or wear.

In implementations, the adhesive patch 602 includes one or more externally worn sensors configured to output one or more physiological signals. For example, the adhesive patch 602 may include at least one sensing electrode 616 (e.g., at least one ECG electrode) in electronic communication with the cardiac monitor 600 and configured to output at least one ECG signal. The at least one sensing electrode 616 may be embedded or integrated into the adhesive patch 602, as shown in FIG. 6. For instance, the adhesive patch 602 may include conductive elements that from the ECG electrode(s) 610 (e.g., a single lead, two leads, etc.) that can be used when recording ECG signals from the surface (e.g., skin contacted directly or through a covering) of a patient's body. The at least one sensing electrode 616 may be coupled to the cardiac monitor 600 by dedicated wiring within the patch 602. In some implementations, the ECG may have a sampling rate in the range from about 250 Hz to about 500 Hz, from about 300 Hz to about 450 Hz, or from about 350 Hz to about 400 Hz, including values and subranges therebetween. In some embodiments, the ECG signal may be sampled after band-pass filtering by a 12-bit analog-to-digital converter (“ADC”). During normal operation, data may be transferred to the remote server 102 “as-is” and can then be used by the remote server 102 for analysis. In some implementations, an internal process allows for real-time evaluation of the ECG signal quality upon each attachment of the wearable cardiac monitoring device 100 to the patient. For example, the cardiac monitor 600, portable gateway 604, and/or remote server 102 may evaluate a signal quality by analyzing the fidelity of an ECG waveform recorded by the cardiac monitor 600. As another example, the cardiac monitor 600 and/or adhesive patch 602 may transmit a testing signal to the patient's body that may be picked up by the ECG electrode(s) 610 to determine whether the ECG electrode(s) 610 are properly adhered to the patient's skin and monitoring the patient's ECG signals.

In some implementations, the remote server 102 and/or wearable cardiac monitoring device 100 (e.g., at the cardiac monitor 600 and/or portable gateway 604) may process the ECG signals to detect an arrhythmia of the patient. Types of arrhythmias detected by the remote server 102 and/or the wearable cardiac monitoring device 100 may include ventricular ectopic beats (VEB), ventricular runs/ventricular tachycardia, bigeminy, supraventricular ectopic beats (SVEB), supraventricular tachycardia, atrial fibrillation, ventricular fibrillation, pauses, 2nd AV blocks, 3rd AV blocks, bradycardia, and/or other types of tachycardia. Additionally, in some implementations, the remote server 102 and/or wearable cardiac monitoring device 100 may perform other processing or analyses of the ECG signal, such as band pass filtering, detecting R-R intervals, detecting QRS intervals, and/or heart rate estimation.

In implementations, the cardiac monitor 600 and/or adhesive patch 602 may include one or more additional sensors configured to sense other biometric signals of the patient 104. For instance, the cardiac monitor 600 may include one or more motion detectors configured to generate motion signals associated with the patient, where the motion signals include information about the activity and/or position (e.g., posture) of the patient 104. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. As another illustration, the cardiac monitor 600 and/or adhesive patch 602 may include another type of sensor or detector, such as a P-wave sensor (e.g., a sensor configured to monitor and isolate P-waves within an ECG waveform), a temperature sensor, an oxygen saturation sensor (e.g., implemented through photoplethysmography, such as through light sources and light sensors configured to transmit light into the patient's body and receive transmitted and/or reflected light containing information about the patient's oxygen saturation), a heart sounds or acoustics sensor, a heart vibrations sensor, and so on. In some implementations, the portable gateway 604 may alternatively or additionally include one or more additional sensors configured to sense other biometric signals of the patient 104. For example, the portable gateway 604 may include a 3-axis accelerometer configured to generate motion signals, including activity and/or position information, associated with the patient 104.

In implementations, the cardiac monitor 600 may include one or more wear state detectors or sensors, as described above. As another illustration, the cardiac monitor 600 may include a radiofrequency (RF) sensor comprising an RF antenna and RF transmitter and receiver such that the cardiac monitor 600 can take bio-impedance measurements of the patient's thorax. RF technology as described herein is available from ZOLL® Medical Corporation (Chelmsford, MA), and makes use of a non-ionizing techniques by illuminating the body with low-power electromagnetic pulses at microwave range frequencies (approximately 0.5-30 GHz, more particularly approximately 0.5-5 GHz, more particularly approximately 0.5-2.5 GHz) and measuring the returned RF signals. The reflected and refracted RF signals enable the system to discern between different tissues based on their electromagnetic properties. Such RF technologies are biologically safe, having electromagnetic power much lower than those from a cellular phone. The RF sensor can be used to determine physiological information such as heart wall motion, respiration, thoracic fluid context (e.g., lung water), certain arrhythmias, heart valve abnormalities, arterial pulse information (e.g. pulse transit time (PTT), pulse arrival times (PAT)), blood pressure readings based on the arterial pulse information. Additionally or alternatively, the cardiac monitor 600 includes a temperature sensing device configured to sense a body temperature of the patient. For example, the temperature sensing device can include an infrared device for determining the body temperature. Additionally or alternatively, the cardiac monitor 600 includes a pulse oximeter configured to sense oxygen saturation information for the patient. Additionally or alternatively, the cardiac monitor 600 includes a respiration accelerometer that is separate from a motion sensor used to measure, for instance, the patient's posture and activity level. To illustrate, the respiratory sensor may be based on the operation of a tri-axis microelectromechanical system (MEMS) accelerometer within the cardiac monitor 600 when mounted on the patient's torso.

Returning to FIG. 6, the portable gateway 604 is configured to receive the signals provided by the monitoring unit (e.g., signals encoding the at least one RF value) and transmit the signals to the remote server 102. Accordingly, the portable gateway 604 may be in wired and/or wireless communication with the cardiac monitor 600 and the remote server 102. As an illustration, the portable gateway 604 may communicate with the cardiac monitor 600 via Ethernet, via Wi-Fi, via near-field communication (NFC), via radiofrequency, and the like. The portable gateway 604 may further communicate with the remote server 102 via cellular networks, via Bluetooth®-to-TCP/IP access point communication, via Ethernet, via Wi-Fi, and the like. As such, the portable gateway 604 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the communications circuitry in the cardiac monitor 600 and/or the portable gateway 604 may be part of an Internet of Things (IoT) and communicate with each other and/or the remote server 102 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

Alternatively, in some implementations, the cardiac monitor 600 may be configured to transmit the sensed or acquired signals (e.g., the at least one RF value) directly to the remote server 102 instead of, or in addition to, transmitting the signals to the portable gateway 604. Accordingly, the cardiac monitor 600 may be in wired or wireless communication with the remote server 102. As an illustration, the cardiac monitor 600 may communicate with the remote server 102 via cellular networks, via Bluetooth®-to-TCP/IP access point communication, via Ethernet, via Wi-Fi, and the like. Further, in some implementations, the wearable cardiac monitoring device 100 may not include the portable gateway 604. In such implementations, the cardiac monitor 600 may perform the functions of the portable gateway 604 described above. Additionally, in such implementations, the cardiac monitor 600 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, as indicated above, the communications circuitry in the cardiac monitor 600 may be part of an IoT and communicate with the remote server 102 via IoT protocols for handling secure (e.g., encrypted) messaging and routing.

The charger 608 includes charging cradles configured to hold and recharge the cardiac monitor 600 and the portable gateway 604. Alternatively, in some implementations, the wearable cardiac monitoring device 100 may not include the portable gateway 604, and accordingly, the charger 608 may be configured to hold the cardiac monitor 600 alone. In some implementations, the cardiac monitor 600 and the portable gateway 604 may have separate chargers, or one or both of the cardiac monitor 600 and the portable gateway 604 may have removable batteries that may be replaced and/or recharged.

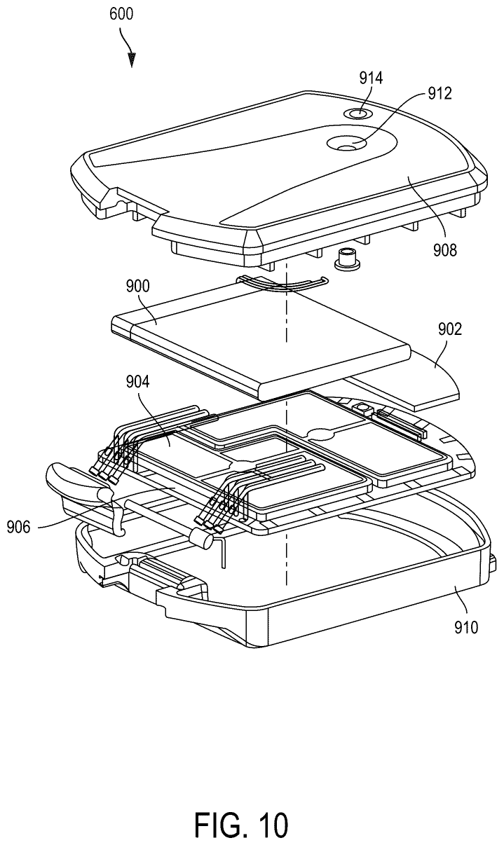

Referring again to the cardiac monitor 600, FIG. 10 provides an exploded view of the cardiac monitor 600, according to some implementations. The exploded view of FIG. 10 illustrates various components of the cardiac monitor 600. For example, the cardiac monitor 600 may include a power source, such as a battery 900. In examples, the battery 900 may be a rechargeable lithium ion battery configured to supply power for at least one month of continuous or near-continuous RF recording and/or recording of other physiological signals, such as ECG signals, from the patient. The cardiac monitor 600 may also include a wireless communications circuit 902; a radiofrequency shield 904 (e.g., a metallic cover, for instance, to prevent interference with the RF processing and other digital circuitry); a digital circuit board 906; and/or the like. The wireless communications circuit 902 may be a Bluetooth® unit, in some implementations, although in addition to or alternatively to the Bluetooth® unit, other modules facilitating other types of communications (e.g., Wi-Fi, cellular, etc.) may be included in the cardiac monitor 600.

In some implementations, the cardiac monitor 600 may be configured to monitor, record, and transmit signals (e.g., signals including the at least one RF value, signals including ECG information) to the portable gateway 604 continuously (e.g., via the wireless communications circuit 902). The cardiac monitor 600 monitoring and/or recording additional data may not interrupt the transmission of already acquired data to the portable gateway 604. As such, in some implementations, both the monitoring/recording and the transmission processes may occur at the same time or nearly the same time. In some implementations, if the cardiac monitor 600 does suspend the monitoring and/or recording of additional data while the cardiac monitor 600 is transmitting already acquired data to the portable gateway 604, the cardiac monitor 600 may then resume monitoring and/or recording of additional data before all of the already-acquired data has been transmitted to the portable gateway 604. To illustrate, the interruption period for the monitoring and/or recording of additional data may be less in comparison to the time it takes the cardiac monitor 600 to transmit the already-acquired data (e.g., the interruption period being between about 0% to 80%, about 0% to about 60%, about 0% to about 40%, about 0% to about 20%, about 0% to about 10%, about 0% to about 5%, including values and subranges therebetween, of the monitoring and/or recording period). This moderate interruption period may facilitate the near-continuous monitoring and/or recording of additional data during transmission of already-acquired physiological data. For example, in one scenario, when a measurement time is about two minutes, any period of suspension or interruption in the monitoring and/or recording of subsequent measurement data may range from a few milliseconds to about a minute. Illustrative reasons for such suspension or interruption of data may include allowing for the completion of certain data integrity and/or other online tests of previously acquired data. In some implementations, if the previous data have problems, the cardiac monitor 600 may notify the patient and/or a remote technician of the problems so that appropriate adjustments can be made.

In some implementations, the cardiac monitor 600 may be configured to monitor, record, and transmit some data in a continuous or near-continuous manner, as discussed above, while monitoring, recording, and transmitting some other data in a non-continuous manner (e.g., periodically, non-periodically, etc.). As an illustration, ECG data may be transmitted to the portable gateway 604 (and, via the portable gateway 604, to the remote server 102) continuously or near-continuously as additional ECG data is being recorded, while other data (e.g., RF data collected by RF sensors) may be transmitted once the measuring process for the other data is completed. In some implementations, monitoring and/or recording of signals by the cardiac monitor 600 may be periodic and, in some implementations, may be accomplished as scheduled (e.g., periodically) without delay or latency during the transmission of already acquired data to the portable gateway 604. For example, the cardiac monitor 600 may take measurements from the patient and transmit the data generated from the measurements to the portable gateway 604 in a continuous manner as described above.

Additionally, in some implementations, the portable gateway 604 may continuously transmit the signals provided by the cardiac monitor 600 to the remote server 102. Thus, for example, the portable gateway 604 may transmit the signals from the cardiac monitor 600 to the remote server 102 with little or no delay or latency. To this end, in the context of data transmission between the wearable cardiac monitoring device 100 and the remote server 102, continuously includes continuous (e.g., without interruption) or near continuous (e.g., within one minute after completion of a measurement and/or an occurrence of an event on the cardiac monitor 600). Continuity may also be achieved by repetitive successive bursts of transmission (e.g., high-speed transmission). Similarly, immediate data transmission may include data transmission occurring or done immediately or nearly immediately (e.g., within one minute after the completion of a measurement and/or an occurrence of an event on the cardiac monitor 600).

Further, in the context of signal acquisition and transmission by the wearable cardiac monitoring device 100, continuously may also include uninterrupted collection of data sensed by the cardiac monitor 600 with clinical continuity. In this case, short interruptions in data acquisition of up to one second several times an hour, or longer interruptions of a few minutes several times a day, may be tolerated and still seen as continuous. As to latency as a result of such a continuous scheme as described herein, the overall amount of response time (e.g., time from when an event onset is detected to when a notification regarding the event is issued) can amount, for example, from about five to fifteen minutes. As such, transmission/acquisition latency may therefore be in the order of minutes.

In some implementations, the bandwidth of the link between the cardiac monitor 600 and the portable gateway 604 may be larger, and in some instances, significantly larger, than the bandwidth of the acquired data to be transmitted via the link (e.g., burst transmissions). Such implementations may ameliorate issues that may arise during link interruptions, periods of reduced/absent reception, etc. In some implementations, when transmission is resumed after an interruption, the resumption may be in the form of last-in-first-out (LIFO). In some implementations, the portable gateway 604 additionally may be configured to operate in a store and forward mode, where the data received from the cardiac monitor 600 is first stored in an onboard memory of the portable gateway 604 and then forwarded to the remote server 102. In some implementations, the portable gateway 604 may function as a pipeline and pass through data from the cardiac monitor 600 immediately to the remote server 102. Further, in some implementations, the data from the cardiac monitor 600 may be compressed using data compression techniques to reduce memory requirements as well as transmission times and power consumption. In some implementations, the link between the portable gateway 604 and the remote server 102 may similarly be larger, and in some instances, significantly larger, than the bandwidth of the data to be transmitted via the portable gateway 604 and the remote server 102.

Referring back to FIG. 10, the components of the cardiac monitor 600 (e.g., the battery 900, communications circuit 902, radiofrequency shield 904, digital circuit board 906, and/or the like) may be provided between a front cover 908 forming an upper surface of the cardiac monitor 600 and a back cover 910 forming a bottom surface of the cardiac monitor 600. For instance, the back cover 910 may be configured to contact the adhesive patch 602, and the front cover 908 may be configured to face away from the patient such that the front cover 908 is accessible when the cardiac monitor 600 is attached to the adhesive patch 602. In some implementations, an indicator light 912 and/or a button 914 may be embedded into the front cover 908 visible through the upper surface. The indicator light 912 may provide feedback on the status of the cardiac monitor 600 and its components, such as the charging and/or power level of the power source of the cardiac monitor 600 (e.g., the battery 900), the attachment level of the cardiac monitor 600 to the adhesive patch 602, the attachment level of the adhesive patch 602 to the surface of the body to which the adhesive patch 602 is attached, etc.

The button 914 may be configured for the patient and/or caregiver to provide feedback to the cardiac monitor 600 and/or, via the cardiac monitor 600, to the remote server 102. For instance, the button 914 may allow the patient and/or caregiver to activate or deactivate the cardiac monitor 600. In some implementations, the button 914 may be used to reset the cardiac monitor 600, as well as pair the cardiac monitor 600 to the portable gateway 604 and initiate communication with the portable gateway 604. In some implementations, the button 914 may allow a user to set the cardiac monitor 600 in an “airplane mode,” for example, by deactivating any wireless communication (e.g., Wi-Fi, Bluetooth®, etc.) with external devices and/or servers, such as the portable gateway 604 and/or the remote server 102.

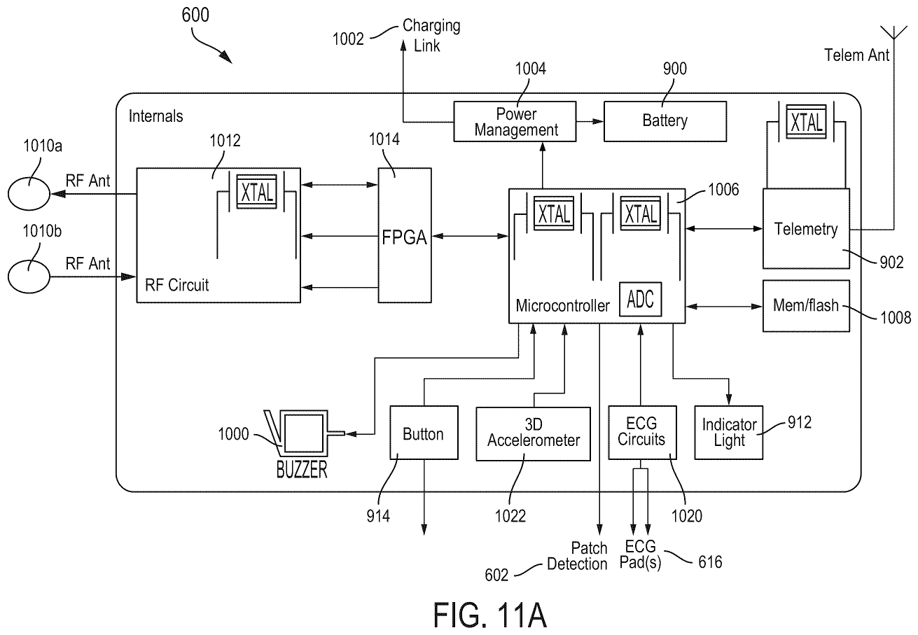

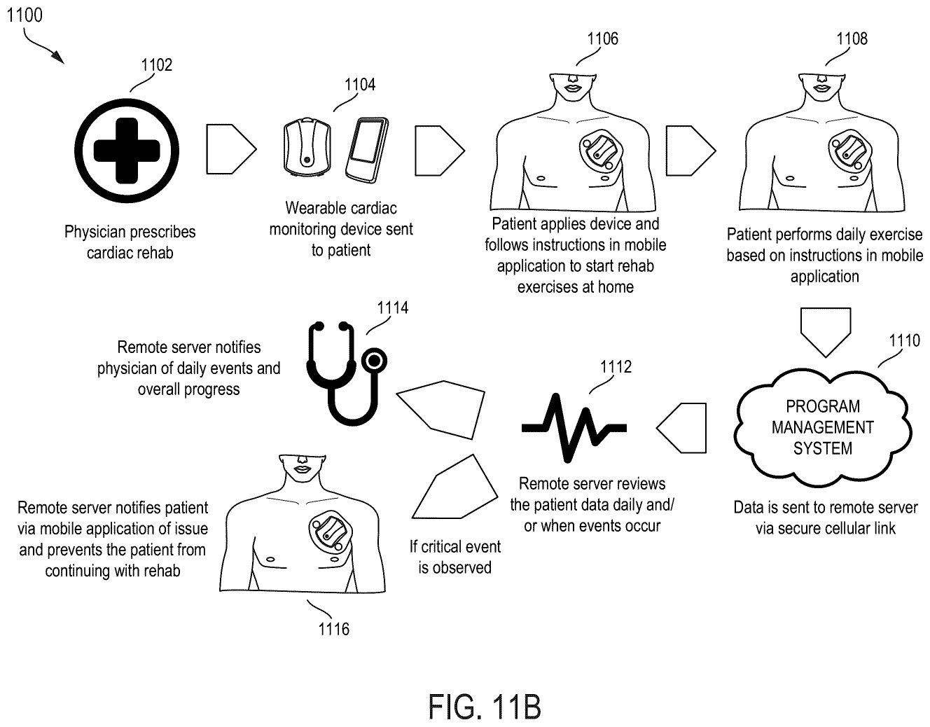

FIG. 11A illustrates an example electronic architecture for the cardiac monitor 600. In some implementations, as shown in FIG. 11A, the cardiac monitor 600 includes one or more external interfaces, either connected to or embedded in the cardiac monitor 600. For example, the cardiac monitor 600 may include the button or switch 914 for activating the cardiac monitor 600, deactivating the cardiac monitor 600, pairing the cardiac monitor 600 with the portable gateway 604, receiving patient input, and/or the like. In some implementations, the cardiac monitor 600 may also include the indicator light 912 and a buzzer 1000 for providing light and/or audio feedback to a user of the cardiac monitor 600 (e.g., in response to the patient activating the button 914 or tapping the cardiac monitor 600 to record that the patient 104 is experiencing symptoms suspected to be related to an arrhythmia).

Further, in some embodiments, the cardiac monitor 600 may be connectable to the ECG pad(s) or electrode(s) 616 coupled to the patient 104 (e.g., connectable to the ECG pad(s) 616 embedded in the adhesive patch 602) and to a charger, such as charger 608, via charging link 1002. For instance, the back cover 910 of the cardiac monitor 600 may include metal contacts configured to connect to the ECG pads 616 when the cardiac monitor 600 is attached to the adhesive patch 602 and to a charging power source when the cardiac monitor 600 is attached to the charger 608. The ECG circuits 1020 may receive signals from the ECG pad(s) 616 when the cardiac monitor 600 is attached to the adhesive patch 602, where the signals received from the ECG pad(s) 610 include ECG waveforms sensed from the patient. Alternatively, or additionally, in some implementations, the cardiac monitor 600 may include an inductive circuit configured to charge the cardiac monitor 600 via a wireless inductive charging link 1002. As shown in FIG. 11A, the charging link 1002 may be coupled to a power management circuit 1004 (e.g., when the cardiac monitor 600 is attached to the charger 608, when the cardiac monitor 600 is placed in proximity to an inductive charging pad), where the power management circuit 1004 is configured to charge an onboard power source such as the battery 900.

Internally, in some implementations, the cardiac monitor 600 may include a microprocessor (e.g., being connected to a separate non-volatile memory, such as memory 1008) or a microcontroller 1006. The microcontroller 1006 stores instructions specifying how measurements (e.g., ECG, accelerometer, etc.) are taken, how obtained data are transmitted, how to relay a status of the cardiac monitor 600, how/when the cardiac monitor 600 can enter a sleep level, and/or the like. In some implementations, the instructions may also specify the conditions for performing certain types of measurements. For example, the instructions may specify that a sensor of the cardiac monitor 600 may not commence measurements (e.g., ECG measurements, respiration rate measurements, etc.) unless the patient using the cardiac monitor 600 is at rest or maintaining a certain posture (e.g., as indicated by data from an accelerometer 1022 of the cardiac monitor 600). As another example, the instructions may identify the conditions that may have to be fulfilled before measurements can commence, such as a sufficient attachment level between the cardiac monitor 600 and the adhesive patch 602 and/or a sufficient attachment level between the adhesive patch 602 and the surface of the body onto which the adhesive patch 602 is attached. In some implementations, the microcontroller 1006 may have internal and/or external non-volatile memory banks (e.g., memory 1008) that can be used for measurement directories and data, scheduler information, and/or a log of actions and errors. This non-volatile memory may allow for retaining data and status information in the case of a total power down.

As discussed above, in some implementations, the cardiac monitor 600 includes at least one RF antenna and RF circuitry for transmitting electromagnetic waves into the body of the patient 104 (e.g., into a thoracic region of the patient 104) and receiving electromagnetic waves that are transmitted through the patient's body and/or scattered or reflected from internal tissues of the patient's body. For example, the at least one RF antenna and RF circuitry may transmit RF waves in a range from about 0.1 GHz to about 5 GHz (e.g., including a small amount above and below this range, such as 10% less than 0.1 GHz and 10% more than 5 GHz, 5% less than 0.1 GHz and 5% more than 5 GHz, and so on). The at least one RF antenna may be flat, printed, set flush against the skin, with or without an interface material, and/or the like. The at least one RF antenna may be in a bow-tie, spiral, monostatic, bistatic, and or like configurations. Further, the RF circuitry is configured to process the received electromagnetic waves so as to determine some properties of the tissues that are on the path of the transmitted and/or received electromagnetic waves. The RF circuitry may process the received electromagnetic waves to produce RF data that the cardiac monitor 600 may transmit to the remote server 102 and/or analyze to determine, for instance, an amount of fluid in the patient's thoracic region. To illustrate, the at least one RF antenna and RF circuitry may transmit electromagnetic waves towards a thoracic region of a patient that includes the patient's lungs and receive electromagnetic waves transmitted through and/or scattered or reflected by the patient's lung tissues. As such, the received electrometric waves may include information about a level of fluid in or near the patient's lungs, which may be contained by the at least one RF value produced by the RF circuitry of the cardiac monitor 600.

In some implementations, the at least one RF antenna and RF circuitry are configured to transmit a low-power signal in an ultra-high frequency band (e.g., 0.1 GHz to 5.0 GHz, 0.5 GHz to 2.1 GHz) at a predetermined rate (e.g., every 10 ms, every 20 ms, every 30 ms, every 40 ms, every 50 ms, etc.). The RF antenna and RF circuitry transmit a burst of a predetermined number of frequencies (e.g., 32 frequencies, 64 frequencies, 128 frequencies) spanning the ultra-high frequency band at the predetermined rate. The at least one RF antenna and RF circuitry receive RF waves transmitted through, scattered by, and/or reflected from the patient 104 for a predetermined amount of time (e.g., about 30 seconds, about 1 minute, about 2 minutes, about 3 minutes, about 5 minutes, about 10 minutes, etc.). In some implementations, the wearable cardiac monitoring device 100 (e.g., at the cardiac monitor 600 and/or the portable gateway 604) and/or the remote server 102 are configured to gate when RF measurements are taken and/or discard certain RF measurements based on the patient's state when the RF measurements were taken. For example, the wearable cardiac monitoring device 100 and/or the remote server 102 may determine whether the patient 104 showed movement above a predetermined threshold while the RF sampling was taking place.

Accordingly, FIG. 11A shows an example embodiment of the cardiac monitor 600 including RF antennae 1010a, 1010b, an RF circuit 1012, and other circuits for controlling the RF circuit 1012 (e.g., field-programmable gate array (FPGA) circuits 1014). In various implementations, the RF antennae 1010a, 1010b are configured to transmit RF waves into the body of a patient 104 to which the cardiac monitor 600 is attached and receive transmitted, scattered, and/or reflected RF waves from the body of the patient 104. Such functionality may be used to monitor, for example, the amount of fluid in the patient's thoracic region, either statically or over time, in accordance with the techniques described herein.

The cardiac monitor 600 may also include or be connected to one or more additional externally worn sensors or detectors, according to various implementations. For example, as shown in FIG. 11A, the cardiac monitor 600 include one or more motion detectors or sensors, such as a 3D accelerometer 1022. The 3D accelerometer 1022 is configured to generate motion data indicative of physical activity performed by the patient 104. As such, using the 3D accelerometer 1022, the cardiac monitor 600 may acquire data on patient movements, patient orientation, patient respiration, and/or the like. The wearable cardiac monitoring device 100 (e.g., at the cardiac monitor 600 and/or portable gateway 604) and/or the remote server 102 may use the acquired accelerometer data to determine one or more patient health values, such as the patient's respiration rate, heart rate, activity rate, posture or orientation, and/or the like. The wearable cardiac monitoring device 100 may also transmit the accelerometer data to the remote server 102 such that the remote server 102 can use the accelerometer data to determine if the patient 104 has met incentive criteria set for the patient 104, as described above.

In implementations, the cardiac monitor 600 may also include detection circuitry configured to detect wear state or wear compliance. For example, in implementations, the cardiac monitor may include a wear compliance detector (e.g., implemented by the microcontroller 1006) similar to the wear compliance detector 530 described above with reference to FIG. 5B.

FIG. 11B illustrates an example process flow 1100 for a patient 104 receiving and using a wearable cardiac monitoring device 100 including a cardiac monitor 600. A physician prescribes cardiac rehab for the patient 104 at step 1102. For example, the physician may prescribe cardiac rehab for 60 to 90 days for the patient 104 upon the patient's discharge from a hospital (e.g., following an adverse cardiac event). The wearable cardiac monitoring device 100 including the cardiac monitor 600 and the portable gateway 604 is sent to the patient 104 (e.g., via overnight mail) at step 1104. The patient 104 applies the wearable cardiac monitoring device 100 at step 1106. The patient 104 also uses a mobile application executed by the portable gateway 604 to start rehab exercises at home. The patient 104 performs the daily exercises based on the instructions in the mobile application at step 1108. The cardiac monitor 600 and/or portable gateway 604 gather data on the patient's execution of the cardiac rehab exercises. This patient data is transmitted to the remote server 102 via a secure cellular link (e.g., by the portable gateway 604) at step 1110. The remote server 102 reviews the patient data daily and/or when events occur at step 1112. Examples of events may include a detected cardiac arrhythmia (e.g., occurring while the patient is exercising or after the patient has exercised), a detected fall, a patient-reported symptom (e.g., reported using the portable gateway 604), and/or the like. The remote server 102 notifies the patient's physician of daily events and the patient's overall progress at step 1114. For instance, the remote server 102 may update a dashboard that shows the patient's daily events and overall progress. As another example, the remote server 102 may send notifications (e.g., email notifications, text or SMS notifications, etc.) to the patient's physician with the patient's daily events and overall progress. If a critical event is detected, such as a cardiac arrhythmia that could develop into a more serious condition, the remote server 102 notifies the patient 104 via the mobile application executed by the portable gateway 604 and prevents the patient 104 from continuing more rehab exercises using the mobile application at step 1116.

With respect to the mobile application executed on the portable gateway 604 guiding the patient 104 through a rehab session, the mobile application activates the cardiac monitor 600 into a scheduled mode for the rehab session and communicates with the cardiac monitor 600 during the rehab session to provide instructions to the patient 104. For example, the portable gateway 604 receives the patient's ECG data and accelerometer data during the rehab session to determine the patient's heart rate, ECG quality indication, step count, step rate, activity/rest state, etc. using similar processes to those described above. The parameters determined by the portable gateway are used to provide the patient 104 with instructions via the mobile application.

As an illustration, the portable gateway 604 determines the patient's heart rate to be the average rate over a time window of length T_{HR}, where T_{HR} is a parameter between 10 and 60 seconds. The portable gateway 604 repeats the heart rate calculation every T_{HR Refresh}, where T_{HR Refresh} is a parameter between 5 to 20 seconds and is short enough to allow for timely instructions to be provided to the patient 104. Every T_{HR Refresh} seconds, the portable gateway 604 outputs the heart rate value that corresponds to the time window of length T_{HR} that started before T_{HR}+T_{Processing} seconds, where T_{Processing} is less than one second. These time windows will overlap. The portable gateway 604 displays the patient's heart rate to the patient 104 via the mobile application. As another illustration, the portable gateway 604 may provide the step count to the patient 104 via the mobile application as the number of steps from the beginning of the rehab session, the number of steps in the last T_{SC} seconds, and the step rate for the last T_{SC} seconds.

Once the patient 104 has completed the rehab session, the portable gateway 604 switches the cardiac monitor 600 back into a continuous mode. Data collected during the rehab session are stored by the cardiac monitor 600 and/or the portable gateway 604 for transmittal to the remote server 102 by the portable gateway 604 (e.g., in first in first out (FIFO) order according to the measurement time). The portable gateway 604 may also transmit backlog patient data sensed by the cardiac monitor 600 before transmitting the patient data from the rehab session and/or refrain from transmitting any patient data sensed by the cardiac monitor 600 following the conclusion of the rehab session until the patient data from the rehab session is transmitted.

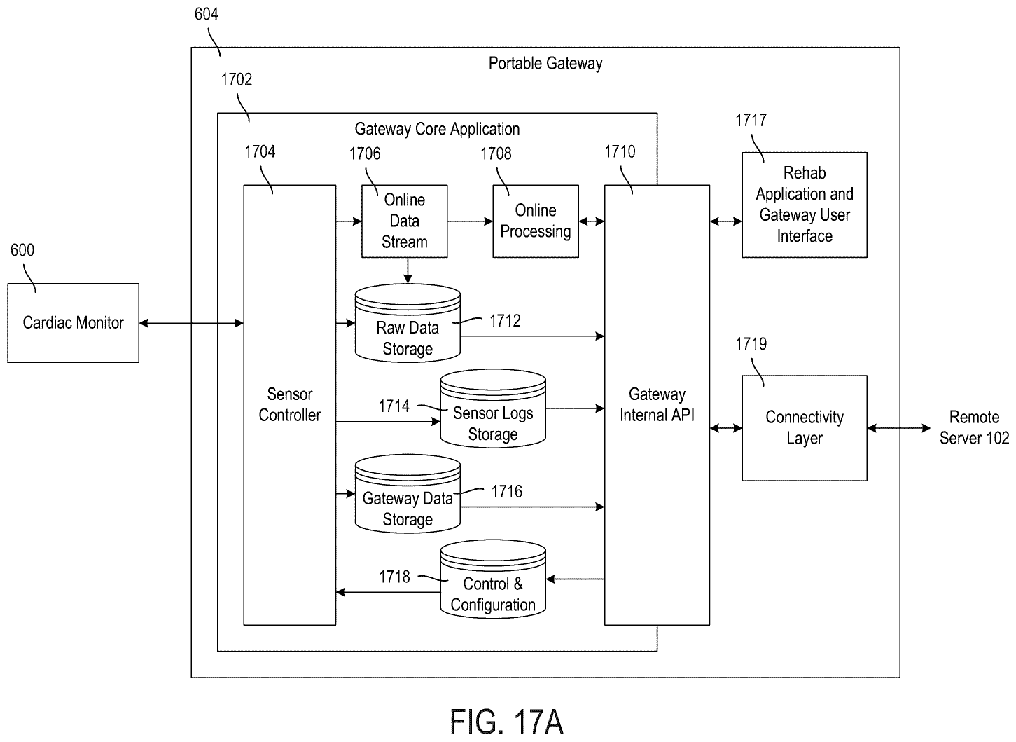

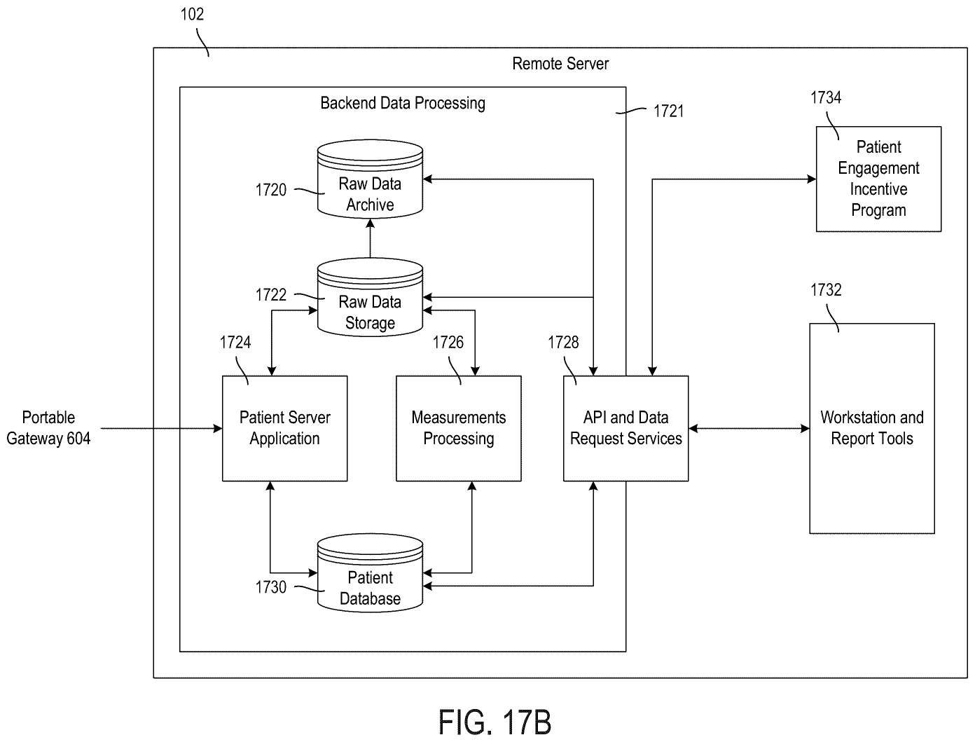

FIGS. 17A and 17B illustrate example system architecture of a wearable cardiac monitoring and rehabilitative system including the cardiac monitor 600 and the portable gateway 604. As described above, after initialization, the cardiac monitor 600 supplied to the patient 104 is applied to an adhesive patch 602 to take measurements such as ECG and accelerometer readings continuously. For each configurable period (e.g., 30 seconds, 60 seconds, 90 seconds, and/or the like), the cardiac monitor 600 collects the measurement data in a file and sends the data file to the portable gateway 604. The cardiac monitor 600 may be able to store a certain amount of measurements while a portable gateway 604 connection is not available, such as 24 hours, 48 hours, 72 hours, 5 days, a week, and/or the like. The cardiac monitor 600 generates a constant connection to the portable gateway 604 while the two devices are in range and may send the measurements files to the portable gateway 604 as soon as the files are created and/or when the connection with the portable gateway 604 is established. Patients may also trigger symptom events by double tapping on the cardiac monitor 600, as described above.

As shown, the portable gateway 604 includes a gateway core application 1702 configured to communicate with the cardiac monitor 600. The portable gateway 604 receives signals and data from the cardiac monitor 600 at a sensor controller 1704 of the portable gateway 604. The portable gateway 604 collects the data received from the cardiac monitor 600, stores the data, and forwards the data to the remote server 102 for analysis (e.g., via a gateway internal API 1710 and connectivity layer 1719). For example, the portable gateway 604 may store raw data from the cardiac monitor 600 in a raw data storage 1712 and sensor logs in a sensor logs storage 1714. The data received from the cardiac monitor 600 may include symptom events and other patient-triggered events, which may be generated by the cardiac monitor 600 and completed by the patient 104 using the portable gateway 604 (e.g., triggered by the patient 104 tapping the cardiac monitor 600 and completed by the patient 104 filling out a questionnaire report on the portable gateway 604). Alternatively, or additionally, a patient-triggered event may be generated and completed by the patient 104 using the portable gateway 604, such as by the patient 104 reporting a symptom by filling out a report on the portable gateway 604. The portable gateway 604 may communicate with the cardiac monitor 600 to generate and receive patient data (e.g., ECG data) related to the reported symptom event. The portable gateway 604 is also configured to forward patient-triggered events to the remote server 102 (e.g., via the gateway internal API 1710 and connectivity layer 1719). The portable gateway storage capacity is large enough to accumulate the data received through the whole prescription time. The portable gateway 604 may also store data and information related to the functioning of the portable gateway 604. For example, the portable gateway 604 may store gateway statuses, logs, and reports in a gateway data storage 1716. In implementations, at least some of the stored gateway data may also be transmitted to the remote server 102.

The sensor controller 1704 may also provide instructions to the cardiac monitor 600, such as instructions to enter and exit certain modes, instructions to transmit certain data to the portable gateway 604, and/or the like. The portable gateway 604 may also receive information, instructions, data, etc. from the remote server 102. As shown, the portable gateway 604 may send data to and receive data from the remote server 102 via a connectivity layer 1719 in communication with the remote server 102. The gateway core application 1702 may receive the data from the remote server 102 through a gateway internal API 1710, which the portable gateway 604 may store, use, and/or transmit to the cardiac monitor 600. As an illustration, the portable gateway 604 may receive control and configuration instructions from the remote server 102, store the control and configuration instructions in a control and configuration database 1718, and forward the control and configuration instructions to the cardiac monitor 600. The portable gateway 604 may also receive instructions from the remote server 102 for the functioning of the portable gateway 604, which may be stored in the control and configuration database 1718.

In implementations, the portable gateway 604 may execute at least some data processing, for example, of the measurements data received from the cardiac monitor 600. For example, an online data stream 1706 may fragment the received data into standard measurements files that are stored on the portable gateway 604 and forwarded to the remote server 102 (e.g., in FIFO order according to the time of measurement). Online processing 1708 may parse and send the patient data to the remote server 102 in a configured window size and overlap.

A rehab application and gateway user interface 1717 may be executed on the portable gateway 604 to facilitate communication with the patient 104. For example, the rehab application and gateway user interface 1717, when executed, may present the patient 104 with interactive user interfaces on the portable gateway 604 to allow the patient 104 to report symptom events, to guide the patient 104 through recommended rehab activities, to provide the patient 104 with information on the status of the cardiac monitor 600 and portable gateway 604, and/or the like. As an illustration, the rehab application and gateway user interface 1717 may be configured to guide the patient 104 through a prescribed exercise program consisting of a sequence of exercise sessions of progressively increasing durations, separated by required resting periods, with daily limits to how many exercise sessions can be completed. Prior to each exercise session, the portable gateway 604 may measure the patient's heart rate and administer a pre-exercise patient self-assessment questionnaire through user interfaces implemented by the rehab application and gateway user interface 1717. During each exercise session, patient parameters (e.g., biometric parameters) may be collected by the portable gateway 604 and the rehab application and gateway user interface 1717. After each exercise session, the rehab application and gateway user interface 1717 may administer a post-exercise self-assessment questionnaire.

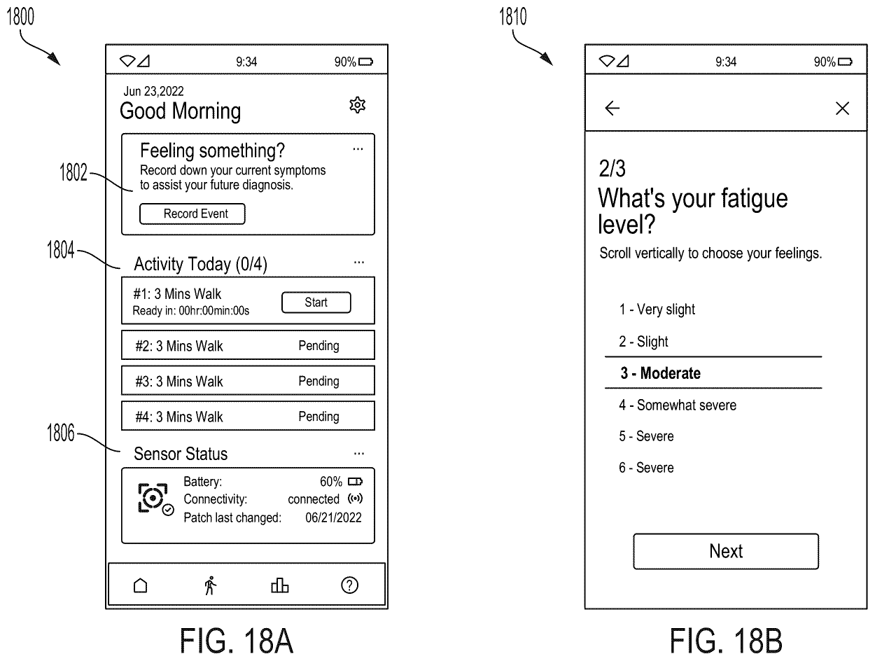

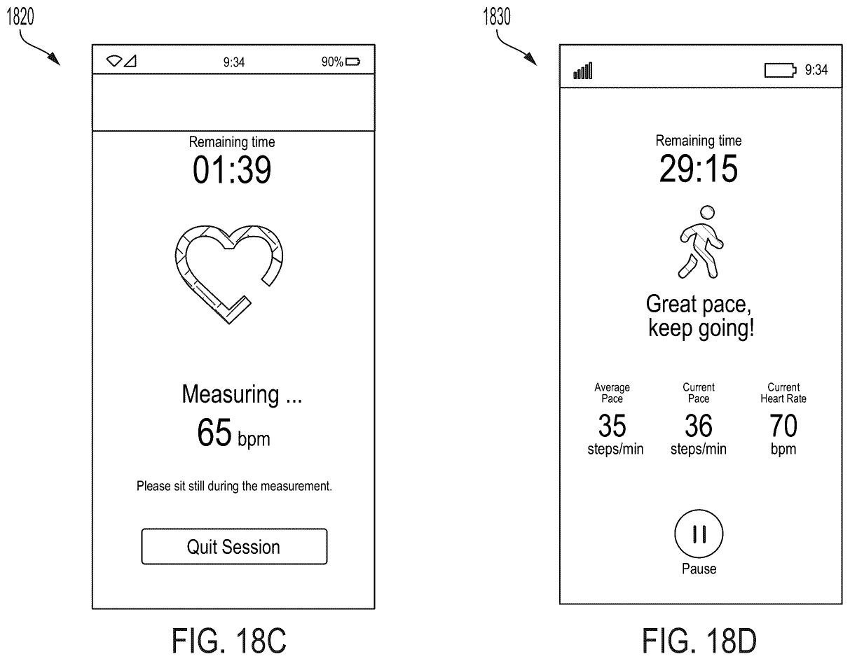

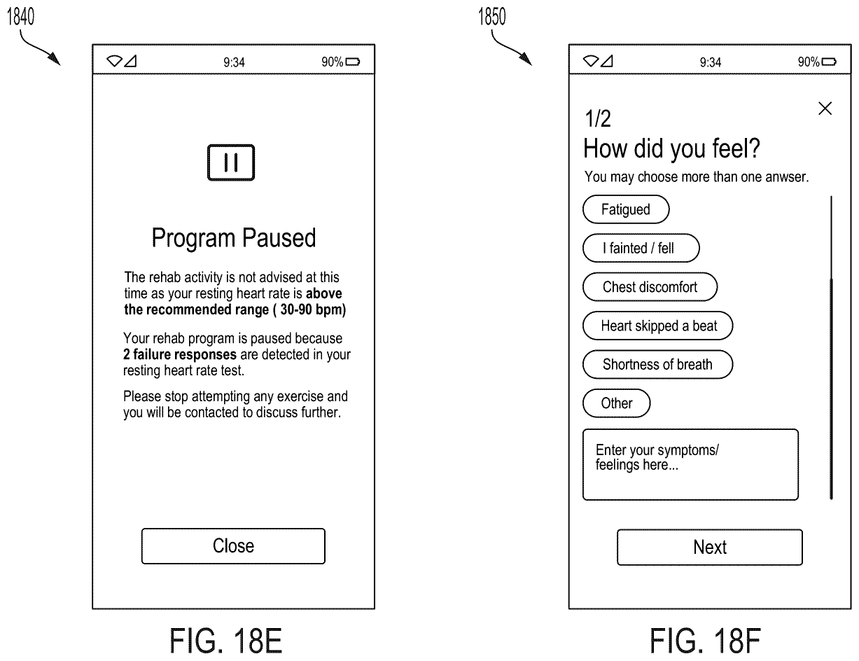

The rehab application and gateway user interface 1717 may communicate with the gateway core application 1702 via the gateway internal API 1710 to gather information about the patient's status and parameters, for instance, while the patient 104 is completing an exercise session as discussed above with respect to FIG. 11B. The gathered information may include the patient's step count, current step rate, current heart rate, and/or the like. The rehab application and gateway user interface 1717 may further use the information about the patient's status to generate and display user interfaces to the patient 104, such as user interfaces instructing the patient 104 on whether to exercise faster, slower, or at the same rate and user interfaces containing the patient's status information, such as their average step rate, current step rate, and current heart rate. Examples of these types of user interfaces are shown in FIGS. 18A-18F.

Referring to FIG. 17B, the remote server 102 may receive data and signals from the portable gateway 604 at a patient server application 1724. A backend data processing section 1721 of the remote server 102 may store raw patient data in a raw data storage 1722 and archive patient data in a raw data archive 1720. The remote server 102 may also process patient data (e.g., to generate patient reports, to implement a patient engagement system as discussed above, etc.) at measurements processing 1726. For instance, the measurements processing 1726 may calculate bio-parameters such as heart rate, respiration rate, patient posture, patient activity, and thoracic fluid levels. The measurements processing 1726 may detect arrhythmia events (e.g., using the patient's ECG data). The measurements processing 1726 may also report patient-triggered events and rehab sessions. Processed patient data may be stored in a patient database 1730. The patient server application 1724 may also manage other aspects of the cardiac monitor 600 and/or portable gateway 604, including managing patient-specific configurations (e.g., the patient's measurement schedule). The patient server application 1724 may also associate received patient data to a client-patient ID (e.g., to keep patients anonymous in the system), initialize the cardiac monitor 600 and/or portable gateway 604, update the cardiac monitor and/or portable gateway 604 with patient-specific parameters, and implement end-of-use procedures.

The backend data processing section 1721 of the remote server 102 may also include API and data request services 1728 to communicate with other functionalities of the remote server 102, such as workstation and report tools 1732 and patient engagement incentive program 1734 functionalities.

FIGS. 18A-18F illustrate example user interfaces that may be displayed to the patient 104 on the portable gateway 604 as part of a rehab application executed by the portable gateway 604. FIG. 18A illustrates an example home screen 1800 that may be displayed to a patient 104 as part of facilitating a rehabilitation program for the patient 104. The home screen 1800 includes a symptom report section 1802 that the patient 104 can use to report a symptom event. The home screen 1800 also includes an activity section 1804 showing the patient 104 the recommended physical activities for the patient 104 to perform as part of their rehab program, including when the patient's next recommended session will be available for them to complete. Additionally, the home screen 1800 includes a sensor status section 1806 configured to display information on the cardiac monitor 600 being used, such as the battery level of the cardiac monitor 600 whether the cardiac monitor 600 is in communication with the portable gateway 604 (e.g., via Blutooth®), whether the portable gateway 604 is in communication with the remote server 102 (e.g., via cellular networks), and the last time the patient 104 changed the adhesive patch 602.

If the patient 104 presses the “start” button on the activity section 1804 to start an exercise session, and there are no blocking conditions for the patient 104 (e.g., no adverse events have been detected for the patient 104 such that the patient 104 has been recommended to stop or pause their rehab program), the patient 104 is directed to pre-session questionnaire screens. The pre-session questions and answers will help the rehab app determine whether the patient 104 should start the exercise session. An example pre-session questionnaire screen 1810 is shown in FIG. 18B, with the patient 104 being asked about their current fatigue level.

After the patient 104 has completed the pre-session questionnaire, the rehab application determines the patient's resting heart rate. As such, the patient 104 may be shown a heart rate screen 1820 instructing the patient 104 to sit still while the portable gateway 604 communicates with the cardiac monitor 600 to determine the patient's resting heart rate. The rehab application also uses the patient's resting heart rate to determine if the patient 104 should begin the exercise session.

If the rehab application determines from the patient's pre-session questionnaire answers and their resting heart rate that the patient 104 should proceed with the exercise session, the rehab application guides the patient 104 to complete the exercise session while receiving feedback. For example, the patient 104 may be shown an exercise screen 1830 instructing the patient to walk at a predetermined pace. The exercise screen 1830 provides the patient 104 with feedback on whether the patient 104 should increase, slow down, or maintain their current pace. The exercise screen 1830 also shows the patient 104 their average pace in steps per minute, current pace in steps per minute, and current heart rate.

If the patient 104 finishes the guided exercise, the patient 104 is presented with a post-session questionnaire, which may be similar to the pre-session questionnaire discussed above with respect to FIG. 18B. The questions and answers may be of a form that allows the rehab application to decide if the exercise activity should be considered successfully completed. However, if during the exercise session, the rehab application determines that the exercise activity should be stopped, the portable gateway 604 alerts the patient 104 of any time delay or block placed on the restart of the exercise session. An explanation of the delay or block is displayed to the patient 104, an example of which is program pause screen 1840 shown in FIG. 18E. In example program pause screen 1840, the portable gateway 604 is alerting the patient 104 that the rehab activity is not advised for the patient 104 at this time because the patient's resting heart rate is above the recommended heart rate range. Additionally, the patient's rehab program has been paused because the patient 104 has had two failure responses in resting heart rate tests. As such, the patient 104 is advised that they will be contacted to determine next steps.

Returning back to the home screen 1800, if the patient 104 presses a button to record a symptom event using the symptom report section 1802, the patient 104 is presented with further screens including a form or forms to collect information about the event and associated symptoms. An example symptom event screen 1850 is shown in FIG. 18F. where the patient 104 is presented with options for types of symptom events that the patient 104 may be experiencing. The patient 104 is also provided a text box to enter in more information about their symptoms and what they are feeling for a caretaker to review.

However, in some implementations, the embodiment of the wearable cardiac monitoring device 100 shown in FIGS. 6-11 may be implemented differently from the configuration shown in these figures. For example, some implementations of the wearable cardiac monitoring device 100 may not include the portable gateway 604, as discussed above. As another example, in some implementations, the wearable cardiac monitoring device 100 may not include an adhesive patch 602. Instead, the cardiac monitor 600 may be mounted on another mechanical implement configured to be removably attached to the patient's body. For instance, the wearable cardiac monitoring device 100 may include a band configured to encircle the patient's chest. The band may be made of an elastic material that compresses the band around the patient's chest to ensure a secure or relatively secure fit where the band does not slip on the patient's chest as the patient moves. The cardiac monitor 600 may accordingly be mounted on the band, such as on a plastic frame similar to the frame of the adhesive patch 602 shown in FIG. 6 or on mating hooks, strips of hook-and-loop cloth, and/or the like.

As another example, in some implementations, the wearable cardiac monitoring device 100 may include more than one cardiac monitor 600 and/or may split the functionalities of the cardiac monitor 600 between multiple devices. As an example, the adhesive patch 602 may be worn on one location of the patient's body (e.g., on the patient's side, such as in the position shown in FIG. 8), with the cardiac monitor 600 mounted on the adhesive patch 602. The cardiac monitor 600/or the adhesive patch 602 may then be in a wired or wireless communication with one or more sensors, such as patch ECG electrodes adhered directly to the patient's skin, worn at a second location of the patient's body (e.g., patch ECG electrodes worn on the patient's chest). As another example, the cardiac monitor 600 may be worn without the adhesive patch 602 at a first location on or near the patient, such as worn on a belt at the patient's waist. The cardiac monitor 600 may then be in a wired or wireless communication with one or more sensors, such as patch ECG electrodes and at least one patch RF antenna adhered directly to the patient's skin, worn at a second location of the patient's body, such as on the patient's chest.

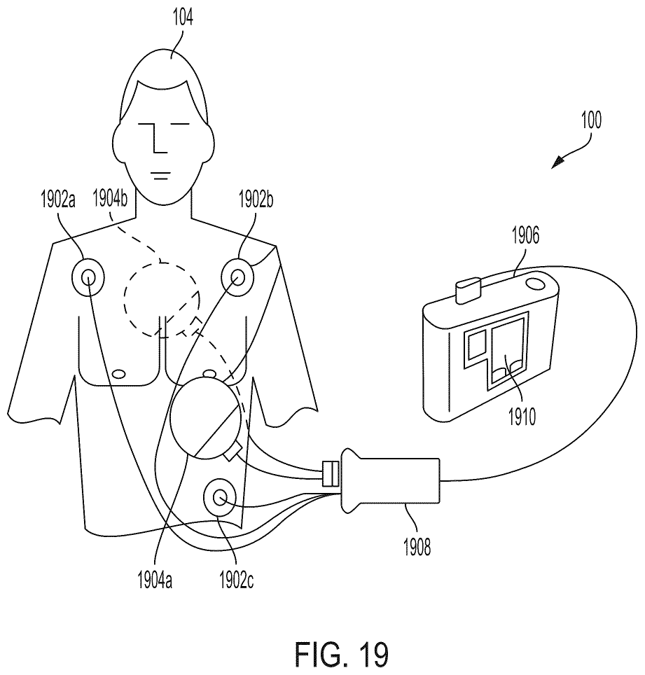

In implementations, the wearable cardiac monitoring device 100 may include electrodes that are disposed on patches adhered to the patient's skin. For example, FIG. 19 shows a wearable cardiac monitoring device 100 that is external, ambulatory, and wearable by the patient 104. The example of the wearable cardiac monitoring device 100 illustrated in FIG. 19 can also be configured in implementations as a therapeutic device configured provide pacing therapy, e.g., to treat bradycardia, tachycardia, and asystole conditions. As shown, the example wearable cardiac monitoring device 100 can include one or more ECG sensing electrodes 1902a, 1902b, and 1902c (e.g., collectively ECG sensing electrodes 1902), therapy electrodes 1904a and 1904b (e.g., collectively therapy electrodes 1904), a medical device controller 1906, and a connection pod 1908. For example, each of these components can be structured and function as similar components of the embodiments of the wearable cardiac monitoring device 100 described above. In implementations, the electrodes 1902 and 1904 can include disposable adhesive electrodes. For instance, the electrodes 1902 and 1904 can include sensing and therapy components disposed on separate sensing and therapy electrode adhesive patches. In implementations, both sensing and therapy components can be integrated and disposed on a same electrodes adhesive patch that is then attached to the patient 104.

As an illustration, the front adhesively attached therapy electrode 1904a attaches to the front of the patient's torso to deliver pacing or defibrillating therapy. Similarly, the back adhesively attachable therapy electrode 1904b attaches to the back of the patient's torso. In an example scenario, at least three ECG adhesively attachable sensing electrodes 1902 can be attached to at least above the patient's chest near the right arm (e.g., electrode 1902a), above the patient's chest near the left arm (e.g., electrode 1902b), and towards the bottom of the patient's chest (e.g., electrode 1902c) in a manner prescribed by a trained professional. In implementations, the example of the wearable cardiac monitoring device 100 shown in FIG. 19 may include additional adhesive therapy electrodes 1904 and/or the patches shown in FIG. 19 may include additional therapy electrodes 1904 on them such that at least two vectors may be formed between the therapy electrodes 1904 of the example wearable cardiac monitoring device 100.

In implementations, the medical device controller 1906 may be configured to function similarly to the medical device controller 406 discussed above. As shown in FIG. 19, the medical device controller 1906 may include a user interface 1910 configured to communicate information with the patient 104. In examples, the patient 104 using the wearable cardiac monitoring device 100 shown in FIG. 19 may be monitored by a caregiver, such as hospital staff or a live-in or visiting caregiver. Additionally, in example, the wearable cardiac monitoring device 100 shown in FIG. 19 may need to be programmed for the patient 104 As such, the user interface 1910 can also be configured to interact with a user other than the patient 104 (e.g., a technician, a clinician, and/or other caregiver) for device-related functions such as an initial baselining (e.g., including performing a baselining therapy session), setting and adjusting patient parameters, and changing the device batteries.

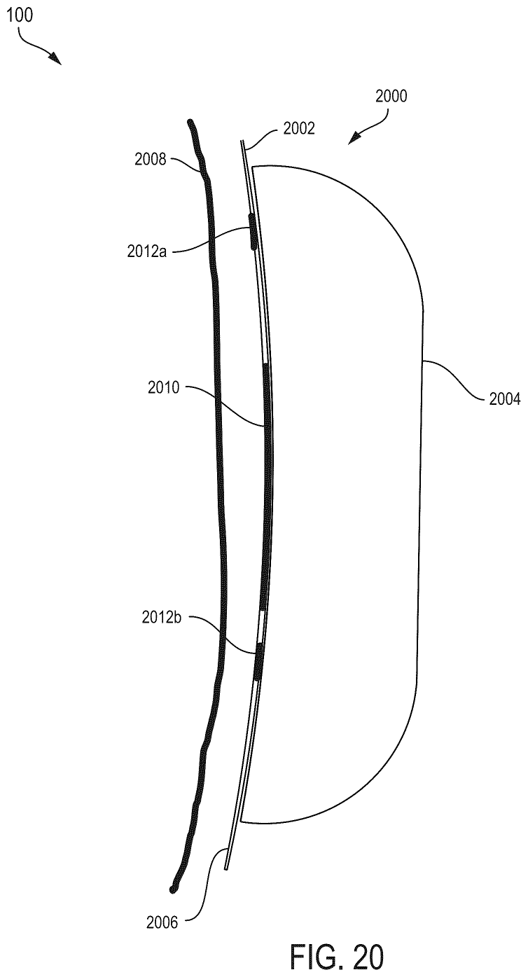

FIG. 20 illustrates another example of a wearable cardiac monitoring device 100. As shown in FIG. 20, the wearable cardiac monitoring device 100 may be or include an adhesive assembly 2000. The adhesive assembly 2000 includes a contoured pad 2002 and a housing 2004 configured to form a watertight seal with a contoured pad 2002. In implementations, the housing 2004 is configured to house electronic components of the adhesive assembly 2000, such as electronic components similar to components described above with respect to the embodiments of the wearable cardiac monitoring device 100 described above. The adhesive assembly 2000 includes a conductive adhesive layer 2006 configured to adhere the adhesive assembly 2000 to a skin surface 2008 of the patient 104. The adhesive layer 2006 may include, for example, a water-vapor permeable conductive adhesive material, such as a material selected from the group consisting of an electro-spun polyurethane adhesive, a polymerized microemulsion pressure sensitive adhesive, an organic conductive polymer, an organic semi-conductive conductive polymer, an organic conductive compound and a semi-conductive conductive compound, and combinations thereof.

The adhesive assembly 2000 also includes at least one therapy electrode 2010 integrated with the contoured pad 2002. In implementations, the adhesive assembly 2000 may include a therapy electrode 2010 that forms a vector with another therapy electrode disposed on another adhesive assembly 2000 adhered to the patient's body and/or with a separate therapy electrode adhered to the patient's body. The adhesive assembly 2000 may also include one or more ECG sensing electrodes 2012 integrated with the contoured pad 2002 (e.g., ECG sensing electrodes 2012a and 2012b). In implementations, the adhesive assembly 2000 may alternatively or additionally be in electronic communication with a separate ECG sensing electrode, such as an adhesive sensing electrode adhered to the patient's body. In examples, as shown in FIG. 20, the therapy electrode(s) 2010 and ECG sensing electrode(s) 2012 may be formed within the contoured pad 2002 such that a skin-contacting surface of each component is coplanar with or protrudes from the patient-contacting face of the contoured pad 2002. Examples of a wearable cardiac monitoring device 100 include an adhesive assembly 2000 are described in U.S. patent application Ser. No. 16/585,344, entitled “Adhesively Coupled Wearable Medical Device,” filed on Sep. 27, 2019, which is hereby incorporated by reference in its entirety.

The embodiments of a wearable cardiac monitoring device 100 described above are examples, and other embodiments of a wearable cardiac monitoring device 100 may be contemplated herein. Additionally features of the wearable cardiac monitoring device 100 shown and described above may be altered, combined, and/or switched out, in some implementations.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-150. (canceled)

151. A wearable cardiac monitoring and rehabilitative system configured to manage patient engagement incentives for ambulatory cardiac patients being monitored for cardiac arrhythmias, the system comprising:
a wearable cardiac monitoring device configured to continuously monitor an ambulatory cardiac patient for one or more arrhythmias, the device comprising
one or more externally worn sensors configured to output one or more physiological signals comprising at least one electrocardiogram (ECG) signal,
one or more motion detectors configured to generate motion data indicative of physical activity performed by the ambulatory cardiac patient, and
wear state detection circuitry configured to detect wear state data of the wearable cardiac monitoring device,
a non-transitory server database; and
one or more server processors in communication with the wearable cardiac monitoring device, the one or more server processors configured to
receive, from a clinician, incentive criteria for a predetermined patient engagement incentive program for the ambulatory cardiac patient,
store, in the server database, a patient incentive data structure for the ambulatory cardiac patient based on the received incentive criteria,
receive from the wearable cardiac monitoring device over a period of time the at least one ECG signal, the motion data indicative of physical activity performed by the ambulatory cardiac patient, and the wear state data,
determine whether the ambulatory cardiac patient has satisfied or partially satisfied the incentive criteria based on at least one of the received motion data or wear state data, and
if the ambulatory cardiac patient has satisfied or partially satisfied the incentive criteria, update the patient incentive data structure to indicate that the ambulatory cardiac patient has earned a complete or partial incentive reward.

152. The system of claim 151, wherein the one or more server processors are further configured to determine a wear state for the wearable cardiac monitoring device, and wherein the wear state comprises an indication of whether the patient wore the wearable cardiac monitoring device.

153. The system of claim 151, wherein determining whether the ambulatory cardiac patient has satisfied or partially satisfied the incentive criteria is further based on the received ECG signal.

154. The system of claim 151, further comprising a clinician-authorized user terminal, wherein the one or more server processors are configured to receive the incentive criteria for the predetermined patient engagement incentive program via the clinician-authorized user terminal.

155. The system of claim 151, wherein the incentive criteria for the predetermined patient engagement incentive program comprise one or more physical activity goals for the ambulatory cardiac patient.

156. The system of claim 155, wherein the one or more physical activity goals comprise at least one of a number of steps for the ambulatory cardiac patient to perform, a number of minutes of physical activity for the ambulatory cardiac patient to perform, or a predetermined combination thereof.

157. The system of claim 155, wherein the one or more server processors are further configured to determine a wear state for the wearable cardiac monitoring device, and wherein the wear state comprises an indication of whether the patient wore the wearable cardiac monitoring device.

158. The system of claim 155, wherein determining whether the ambulatory cardiac patient has satisfied or partially satisfied the incentive criteria is further based on the received ECG signal.

159. The system of claim 151, wherein the incentive criteria comprise a first set of incentive criteria, and wherein the one or more server processors are further configured to
receive, from the clinician, a second set of incentive criteria for the predetermined patient engagement incentive program for the ambulatory cardiac patient; and
update, in the server database, the patient incentive data structure for the ambulatory cardiac patient based on the received second set of incentive criteria.

160. The system of claim 159, wherein the one or more server processors are further configured to
determine whether the ambulatory cardiac patient has satisfied or partially satisfied the second set of incentive criteria; and
if the ambulatory cardiac patient has satisfied or partially satisfied the second set of incentive criteria, update the patient incentive data structure to indicate that the ambulatory cardiac patient has earned a complete or partial incentive reward.

161. The system of claim 160, wherein the second set of incentive criteria for the predetermined patient engagement incentive program comprise the ambulatory cardiac patient acknowledging a message sent to the ambulatory cardiac patient, and wherein the one or more server processors are further configured to
receive message acknowledgement data for the ambulatory cardiac patient; and
determine whether the ambulatory cardiac patient has satisfied or partially satisfied the second set of incentive criteria based on the message acknowledgement data.

162. The system of claim 160, wherein the second set of incentive criteria for the predetermined patient engagement incentive program comprise the ambulatory cardiac patient attending an appointment with a caregiver, and wherein the one or more server processors are further configured to
receive appointment data for the ambulatory cardiac patient; and
determine whether the ambulatory cardiac patient has satisfied or partially satisfied the second set of incentive criteria based on the appointment data.

163. The system of claim 160, wherein the second set of incentive criteria for the predetermined patient engagement incentive program comprise the ambulatory cardiac patient sharing information related to at least one of a health status of the ambulatory cardiac patient or a status of the wearable cardiac monitoring device with a caregiver, and wherein the one or more server processors are further configured to
receive information sharing data for the ambulatory cardiac patient; and
determine whether the ambulatory cardiac patient has satisfied or partially satisfied the second set of incentive criteria based on the information sharing data.

164. The system of claim 151, wherein the one or more server processors are further configured to assign at least one predetermined weight to the incentive criteria.

165. The system of claim 164, wherein at least one of a size or a type of the complete or partial incentive reward depends on the at least one predetermined weight of the incentive criteria.

166. The system of claim 164, wherein the weight assigned to each incentive criterion is associated with a risk score for the ambulatory cardiac patient's health if the ambulatory cardiac patient does not satisfy the incentive criterion.

167. The system of claim 151, wherein the one or more server processors are further configured to determine whether the ambulatory cardiac patient has satisfied or partially satisfied the incentive criteria by determining whether the ambulatory cardiac patient has satisfied a predetermined proportion of the incentive criteria that is less than a total of the incentive criteria.

168. The system of claim 151, wherein the wearable cardiac monitoring device further comprises a garment configured to be worn about a torso of the ambulatory cardiac patient, wherein the one or more externally worn sensors and the one or more motion detectors are configured to be mounted on the garment.

169. The system of claim 151, wherein the wearable cardiac monitoring device further comprises at least one treatment electrode configured to deliver a cardioversion/defibrillation shock to the ambulatory cardiac patient.

170. The system of claim 151, wherein the wearable cardiac monitoring device comprises an adhesive patch configured to be worn on skin of the ambulatory cardiac patient for an extended period of time and a cardiac monitor configured to be mounted on the adhesive patch.

171. A wearable cardiac monitoring and rehabilitative system configured to monitor patient engagement among ambulatory cardiac patients being monitored for cardiac arrhythmias, the system comprising:
a wearable cardiac monitoring device configured to continuously monitor an ambulatory cardiac patient for one or more arrhythmias, the device configured to generate one or more of an electrocardiogram (ECG) signal, motion data indicative of physical activity performed by the ambulatory cardiac patient, or wear state data of the wearable cardiac monitoring device;
a non-transitory server database; and
one or more server processors in communication with the wearable cardiac monitoring device, the one or more server processors configured to
receive, from a clinician, incentive criteria for a predetermined patient engagement incentive program for the ambulatory cardiac patient,
store, in the server database, a patient incentive data structure for the ambulatory cardiac patient based on the received incentive criteria,
receive from the wearable cardiac monitoring device over a period of time the one or more of the ECG signal, the motion data indicative of physical activity performed by the ambulatory cardiac patient, or the wear state data of the wearable cardiac monitoring device,
update, based on the received one or more of the ECG signal, motion data, or wear state data, the patient incentive data structure to indicate the ambulatory cardiac patient's progress towards earning incentive rewards,
retrieve, from the server database, incentive rewards progress information of a predetermined cohort of other ambulatory cardiac patients, and
provide an output indicating the ambulatory cardiac patient's progress towards earning incentive rewards relative to the incentive rewards progress information of the predetermined cohort of other ambulatory cardiac patients.

172. The system of claim 171, wherein the output comprises a visual representation indicating the ambulatory cardiac patient's progress towards earning incentive rewards relative to the incentive rewards progress information of the predetermined cohort of other ambulatory cardiac patients.

173. The system of claim 172, wherein the one or more server processors are further configured to transmit the visual representation to a patient user device.

174. The system of claim 172, wherein the one or more server processors are further configured to transmit the visual representation to a clinician-authorized user terminal.

175. The system of claim 171, wherein the output indicating the ambulatory cardiac patient's progress towards earning incentive rewards relative to the incentive rewards progress information of the predetermined cohort of other ambulatory cardiac patients comprises a trend of incentive rewards earned by the ambulatory cardiac patient over a time period relative to a trend of incentive rewards earned by the predetermined cohort of other ambulatory cardiac patients over the time period.

176. The system of claim 171, wherein the one or more server processors are further configured to
receive, from the clinician, aggregate incentive criteria for a patient population comprising the ambulatory cardiac patient, wherein the aggregate incentive criteria define an aggregate goal for the patient population; and
store, in the server database, an aggregate incentive data structure for the patient population based on the aggregate incentive criteria.

177. The system of claim 176, wherein the one or more server processors are further configured to
update, based on one or more of motion data of the patient population or wear state data of the patient population, the aggregate incentive data structure to indicate the patient population's progress towards earning incentive rewards; and
provide an aggregate output indicating the patient population's progress towards completing the aggregate goal.

178. The system of claim 171, wherein the one or more server processors are further configured to identify the predetermined cohort of other ambulatory cardiac patients.

179. The system of claim 178, wherein the one or more server processors are further configured to identify the predetermined cohort of other ambulatory cardiac patients based on a demographic shared between the ambulatory cardiac patient and the cohort of other ambulatory cardiac patients.

180. The system of claim 178, wherein the one or more server processors are further configured to identify the predetermined cohort of other ambulatory cardiac patients based on a date range during which the ambulatory cardiac patient and the cohort of other ambulatory cardiac patients received their respective wearable cardiac monitoring devices.
